SEQUENCE LISTING (54 sequences)

SEQ ID NO: 1 - protein, 475 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 2 - protein, 475 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSGCGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 3 - protein, 154 aa, Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 4 - protein, 11 aa, synthetic construct
SGGSSGSSGGS

SEQ ID NO: 5 - protein, 165 aa, synthetic construct
SGGSSGSSGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 6 - protein, 640 aa, synthetic construct
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELLSGGSSGSSGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 7 - protein, 640 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSGCGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELLSGGSSGSSGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 8 - RNA, 1425 nt, synthetic construct
  source 1..1425: mol_type = other RNA
atggaattgctgatcttgaaagccaacgccatcacaaccatattgacagcagtaaccttttgtttcgcaagtggtcaaaatattactgaagaattctaccaatcaacgtgtagtgcggtctcaaagggctatcttggcgccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggacttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccataccaaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttg

SEQ ID NO: 9 - RNA, 1425 nt, synthetic construct
  source 1..1425: mol_type = other RNA
atggaattgctgatcttgaaagccaacgccatcacaaccatattgacagcagtaaccttttgtttcgcaagtggtcaaaatattactgaagaattctaccaatcaacgtgtagtgcggtctcaaagggctatctttcagccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggagttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccatatccaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttg

SEQ ID NO: 10 - RNA, 462 nt, Aquifex aeolicus
  source 1..462: mol_type = other RNA
atgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgagg

SEQ ID NO: 11 - RNA, 33 nt, synthetic construct
  source 1..33: mol_type = other RNA
tccggcggaagttctggctctagtgggggaagc

SEQ ID NO: 12 - RNA, 495 nt, synthetic construct
  source 1..495: mol_type = other RNA
tccggcggaagttctggctctagtgggggaagcatgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgagg

SEQ ID NO: 13 - RNA, 1920 nt, synthetic construct
  source 1..1920: mol_type = other RNA
atggaattgctgatcttgaaagccaacgccatcacaaccatattgacagcagtaaccttttgtttcgcaagtggtcaaaatattactgaagaattctaccaatcaacgtgtagtgcggtctcaaagggctatcttggcgccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggacttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccataccaaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttgtccggcggaagttctggctctagtgggggaagcatgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgagg

SEQ ID NO: 14 - RNA, 1920 nt, synthetic construct
  source 1..1920: mol_type = other RNA
atggaattgctgatcttgaaagccaacgccatcacaaccatattgacagcagtaaccttttgtttcgcaagtggtcaaaatattactgaagaattctaccaatcaacgtgtagtgcggtctcaaagggctatctttcagccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggagttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccatatccaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttgtccggcggaagttctggctctagtgggggaagcatgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgagg

SEQ ID NO: 15 - RNA, 13 nt, synthetic construct
  source 1..13: mol_type = other RNA
attgtgctgcatc

SEQ ID NO: 16 - RNA, 52 nt, Homo sapiens
  source 1..52: mol_type = mRNA
aactggagactgggtgaaagtgactaccagaaagtgaggaagcctaaataaa

SEQ ID NO: 17 - RNA, 2143 nt, synthetic construct
  source 1..2143: mol_type = other RNA
aggattgtgctgcatcaagcttgccgccaccatggaattgctgatcttgaaagccaacgccatcacaaccatattgacagcagtaaccttttgtttcgcaagtggtcaaaatattactgaagaattctaccaatcaacgtgtagtgcggtctcaaagggctatcttggcgccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggacttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccataccaaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttgtccggcggaagttctggctctagtgggggaagcatgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgaggtgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - RNA, 2143 nt, synthetic construct
  source 1..2143: mol_type = other RNA
aggattgtgctgcatcaagcttgccgccaccatggaattgctgatcttgaaagccaacgccatcacaaccatattgacagcagtaaccttttgtttcgcaagtggtcaaaatattactgaagaattctaccaatcaacgtgtagtgcggtctcaaagggctatctttcagccctgcgcaccggctggtatacatccgtcataacgatcgaactgagcaacatcaaagagaacaagtgcaatgggactgacgcaaaagttaagcttatcaaacaggagctggacaaatacaaaaatgctgtcacggagttgcagctcttgatgcaaagcacgccggcgaccggctctggaagtgcgattgcatcaggtgttgcagtctgtaaggtattgcacctcgaaggtgaagttaataagataaagtccgcccttctttccactaacaaggccgtagtgtctttgagcggttgtggggtatccgtattgacatttaaagtgcttgatctcaagaactacatcgacaaacagctcttgcccatcctcaacaaacaaagctgctccatatccaacatagaaactgtgattgagttccaacagaaaaacaataggctccttgaaataacgcgggagttttccgttaatgcgggggtaactactccagttagcacgtatatgctcactaatagtgaactcttgtccctcataaatgatatgccgattaccaatgaccagaaaaaactcatgagcaataacgtccagattgtaagacaacagtcctacagtataatgtgtataatcaaggaggaagtcttggcttatgtggttcagcttccactctatggcgtgatagacacaccctgttggaagcttcatactagcccactctgcactacaaacacaaaggaggggagtaatatctgtcttacccgcacagaccgagggtggtattgcgataatgcggggtccgtaagcttcttcccccaagcggagacttgcaaggtacagagtaaccgagtcttctgcgatactatgaatagtcgcacactcccaagcgaggtaaatttgtgcaacgttgatatatttaatcccaaatacgactgtaagattatgacttccaagacagacgtgtctagttctgtcattacttctctcggtgcaattgtttcctgttatgggaagacaaagtgtactgcgtcaaataagtgtcgaggcataataaaaactttctcaaatggatgcgactacgtcagcaataaaggggttgacacggtttcagtgggtaataccctttactatgtcaacaagcaagaaggaaagagcttgtatgtcaagggagagccaatcatcaatttctatgacccccttgtatttccctctgatgagttcgacgcttccataagccaggtgaatgaaaaaattaatcaaagtctggctttcattcggaaatcagacgaacttttgtccggcggaagttctggctctagtgggggaagcatgcagatttatgagggcaagctcacagcagaaggcttgagattcggcattgtagcctctcgcttcaaccacgcactcgttgaccgccttgtcgaaggtgctattgactgtatcgttcggcatgggggtcgcgaggaagatataactctggttagagttcctggtagttgggagatccccgttgcggcaggagagctggctagaaaagaagacatcgacgcagtaatcgccatcggcgtattgattcgcggggctaccccgcatttcgattacatagcgtctgaagtaagtaagggactggcaaatctctcattggagttgcgaaagccgattacttttggagtcatcaccgccgacaccctggaacaagccattgaacgggcgggtaccaagcatggtaataagggctgggaagcggcgctcagtgccattgaaatggcgaacctgtttaaatcactgaggtgatgataataggactagtggatccaactggagactgggtgaaagtgactaccagaaagtgaggaagcctaaataaacctagcgtacgtaaaaaatggaaagaacctagcgtacgaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - protein, 574 aa, Human orthopneumovirus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 20 - RNA, 31 nt, Homo sapiens
  source 1..31: mol_type = mRNA
attctgctgtttgctccttgccaggttcaac

SEQ ID NO: 21 - RNA, 28 nt, Homo sapiens
  source 1..28: mol_type = mRNA
aaaatctcttgattcctagtctctcgat

SEQ ID NO: 22 - RNA, 49 nt, Homo sapiens
  source 1..49: mol_type = mRNA
atcctcaaccaagcgccgcagacggtggccgagcgggggaccgggaagc

SEQ ID NO: 23 - RNA, 61 nt, Homo sapiens
  source 1..61: mol_type = mRNA
gtcccgcagtcggcgtccagcggctctgcttgttcgtgtgtgtgtcgttgcaggccttatt

SEQ ID NO: 24 - RNA, 200 nt, Homo sapiens
  source 1..200: mol_type = mRNA
gtcgctaaagcccctccagtcccagctttgaggctgggcccaggatcactctacagcctgcctcctgggttttccctgggggccgcaatctggctcctgcaggcctttctgtggtcttcctttatccaggctttctgctctcagccttgccttccttttttctgcgactcctgggctgcccctttcacttgcaaaataaa

SEQ ID NO: 25 - RNA, 284 nt, Homo sapiens
  source 1..284: mol_type = mRNA
gacgcccctccctcctctggagtctacgttctccagcccacacccctccaaagccccacctttaagtcctctcttctctattcctgaccctccctacctgaggcctatctgagactataactgtcatctagccccttcgaggttgccccttccccatctccatttcacacaggtcctgaaagtctgtgtccaattatgcactggccacccaacagcaccaattgtacattccctgcatccaatctgctcagcaggccctaagatgccttgagtctttaatccca

SEQ ID NO: 26 - RNA, 187 nt, Homo sapiens
  source 1..187: mol_type = mRNA
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 27 - protein, 4 aa, synthetic construct
GGGS

SEQ ID NO: 28 - protein, 5 aa, synthetic construct
GGGGS

SEQ ID NO: 29 - protein, 4 aa, synthetic construct
GGSG

SEQ ID NO: 30 - protein, 4 aa, synthetic construct
GSGG

SEQ ID NO: 31 - protein, 4 aa, synthetic construct
SGGG

SEQ ID NO: 32 - protein, 4 aa, synthetic construct
SSGG

SEQ ID NO: 33 - protein, 4 aa, synthetic construct
SSSG

SEQ ID NO: 34 - protein, 4 aa, synthetic construct
SAGS

SEQ ID NO: 35 - protein, 5 aa, synthetic construct
GGGSG

SEQ ID NO: 36 - protein, 11 aa, synthetic construct
TGGGGSGGGGS

SEQ ID NO: 37 - protein, 10 aa, synthetic construct
GGGGSGGGGS

SEQ ID NO: 38 - protein, 549 aa, Human orthopneumovirus
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 39 - protein, 438 aa, Human orthopneumovirus
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 40 - protein, 84 aa, Human orthopneumovirus
QNITEEFYQSTCSAVSKGYLSALRTGWYTSVITIELSNIKENKCNGTDAKVKLIKQELDKYKNAVTELQLLMQSTPPTNNRARR

SEQ ID NO: 41 - protein, 25 aa, Human orthopneumovirus
MELLILKANAITTILTAVTFCFASG

SEQ ID NO: 42 - protein, 27 aa, Human orthopneumovirus
ELPRFMNYTLNNAKKTNVTLSKKRKRR

SEQ ID NO: 43 - RNA, 31 nt, synthetic construct
  source 1..31: mol_type = other RNA
aggattgtgctgcatcaagcttgccgccacc

SEQ ID NO: 44 - RNA, 114 nt, Homo sapiens
  source 1..114: mol_type = mRNA
tgatgataataggactagtggatccaactggagactgggtgaaagtgactaccagaaagtgaggaagcctaaataaacctagcgtacgtaaaaaatggaaagaacctagcgtac

SEQ ID NO: 45 - protein, 370 aa, synthetic construct
GSAIASGVAVCKVLHLEGEVNKIKSALLSTNKAVVSLSGCGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 46 - protein, 370 aa, synthetic construct
GSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 47 - protein, 78 aa, synthetic construct
QNITEEFYQSTCSAVSKGYLSALRTGWYTSVITIELSNIKENKCNGTDAKVKLIKQELDKYKNAVTELQLLMQSTPAT

SEQ ID NO: 48 - protein, 78 aa, synthetic construct
QNITEEFYQSTCSAVSKGYLGALRTGWYTSVITIELSNIKENKCNGTDAKVKLIKQELDKYKNAVTDLQLLMQSTPAT

SEQ ID NO: 49 - protein, 513 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 50 - protein, 27 aa, Escherichia phage T4
GYIPEAPRDGQAYVRKDGEWVLLSTFL

SEQ ID NO: 51 - protein, 163 aa, Helicobacter pylori
DIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 52 - protein, 173 aa, synthetic construct
ESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 53 - protein, 24 aa, Tomato bushy stunt virus
INHVGGTGGAIMAPVAVTRQLVGS

SEQ ID NO: 54 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

(19) United States
(12) Patent Application Publication
(10) Pub. No.: US 2025/0041400 A1
(43) Pub. Date: Feb. 6, 2025

(54) RSV VACCINE

(71) Applicant: AstraZeneca AB, Södertälje (SE)

(72) Inventors: Jason Paul LALIBERTE, Wilmington, DE (US); Wade Stanton BLAIR, Wilmington, DE (US); Cheyne KUROKAWA, Wilmington, DE (US)

(21) Appl. No.: 18/776,466

(22) Filed: Jul. 18, 2024

Related U.S. Application Data
(60) Provisional application No. 63/514,277, filed on Jul. 18, 2023.

(51) Int. Cl. A61K 39/155; A61K 9/127; A61K 9/51; A61K 39/00; A61P 31/14; C12N 7/00

(52) U.S. Cl. CPC A61K 39/155; A61K 9/127; A61K 9/5123; A61P 31/14; C12N 7/00; A61K 2039/53; A61K 2039/55555; C12N 2760/18534; C12N 2760/18551

(57) ABSTRACT

Provided herein is a vaccine against RSV. The vaccine comprises an mRNA encoding a stabilised prefusion RSV F protein immunogen linked to a scaffold based on lumazine synthase.

### CROSS-REFERENCE TO RELATED APPLICATIONS

This application claims priority to U.S. Application No. 63/514,277, filed Jul. 18, 2023, which is incorporated herein by reference.

### REFERENCE TO SEQUENCE LISTING SUBMITTED ELECTRONICALLY

The content of the electronically submitted sequence listing (Name: Sequence Listing.xml; Size: 66,742 bytes; and Date of Creation: Jul. 16, 2024) filed with the application is incorporated herein by reference in its entirety.

### FIELD

Provided herein is an mRNA molecule for an RSV vaccine, and an RSV mRNA vaccine comprising the mRNA molecule.

### BACKGROUND

Respiratory syncytial virus (RSV) is an enveloped non-segmented negative-strand RNA virus in the family Paramyxoviridae, genus Pneumovirus. A seasonal virus, it is the most common cause of bronchiolitis and pneumonia among children in their first year of life. RSV also causes repeated infections including severe lower respiratory tract disease, which may occur at any age, especially among the elderly or those with compromised cardiac, pulmonary, or immune systems. There are two antigenic subtypes of RSV: RSV A and RSV B, both of which circulate during epidemics with their relative prevalence varying between seasons. At present, there are no targeted treatments for RSV infections, and there is a need for vaccines which provide protection against RSV infection and disease.

The RSV F protein is a trimeric surface glycoprotein anchored to the viral envelope, and mediates viral fusion to facilitate viral entry into target cells. The protein is initially expressed as a single polypeptide precursor, designated F0. F0 trimerises in the endoplasmic reticulum and is processed intracellularly by a furin protease at two conserved sites, generating F1, F2 and Pep27 polypeptides. The Pep27 polypeptide is excised and does not form part of the mature F protein. The F2 polypeptide originates from the N-terminal portion of the F₀ precursor and links to the F1 polypeptide via two disulfide bonds. The F1 polypeptide anchors the mature F protein in the viral envelope via a transmembrane domain, which is linked to a ˜24 amino acid cytoplasmic tail. Three protomers of the F1-F2 heterodimer assemble to form a mature F protein, which adopts a metastable “pre-fusion” conformation that undergoes a spontaneous conformational change into the “post-fusion” conformation that fuses the viral and target-cell membranes. The “pre-fusion” conformation is known as “pre-F”, and the “post-fusion” confirmation as “post-F”.

Several neutralisation epitopes are not available on post-fusion F protein, and it has been found that the level of pre-F-specific antibodies determines the magnitude of RSV neutralising activity in human sera (Ngwuta et al., Science Translational Medicine 7(309): 309ra162, 2015). In the context of a vaccine, stabilisation of the RSV F protein in the pre-F conformation is therefore advantageous, and RSV F proteins stabilised in the prefusion conformation that produce an enhanced neutralising immune response in animal models compared to the native protein have been identified.

An early example of a stabilised pre-F RSV F protein is the DS-Cav1 protein (McLellan et al., Science 342: 592-598, 2013). DS-Cav1 comprises the mutations 155C, 290C, 190F and 207L, with the introduced cysteine residues forming a disulphide bond and the other mutations filling a cavity in the pre-F structure. The trimeric structure was stabilised with a foldon trimerization motif. To date, two RSV vaccines have been approved: GSK's Arexvy and Pfizer's Abrysvo, both of which have been approved for adults aged 60+. At least one of these is a protein subunit vaccine based on the DS-Cav1 protein.

Work to develop improved RSV antigens has continued, including based on DS-Cav1. One approach which has been tried utilises nanoparticles formed of 24 subunits of a fusion protein comprising the Helicobacter pylori ferritin joined to the C-terminus of a modified, single chain DS-Cav1 in which the furin cleavage sites and p27 regions were deleted, the cysteine mutations were reversed and glycan sites added to mask non-neutralising and poorly neutralising epitopes (Swanson et al., Science Immunology 5: eaba6466, 2020). Another approach was based on iterative cycles of structure-based design, which yielded multiple candidate antigens including various additional mutations and single chain DS-Cav1 derivatives (Joyce et al., Nature Structural and Molecular Biology 23(9): 811-820, 2016; WO 2017/172890).

In recent years, messenger RNA (mRNA) vaccine technology has reached prominence, particularly due to the Moderna and Pfizer/BioNTech Covid-19 vaccines which were used in vaccination campaigns around the world. Rather than directly injecting an antigen into a patient, in an mRNA vaccine an mRNA molecule encoding an antigen is administered to the patient. The antigen is expressed from the mRNA in a small number of cells in the patient's body, resulting in the antigen being presented to the immune system which mounts an immune response.

### SUMMARY

The present inventors have developed an mRNA RSV vaccine utilising a 2nd generation pre-F F protein antigen based on DS-Cav1. The antigen is provided in the form of a fusion protein further comprising a lumazine synthase scaffold. The fusion protein constitutes a nanoparticle subunit (and the terms “fusion protein” and “nanoparticle subunit” are used interchangeably herein) which multimerises to form a nanoparticle. As shown in the examples below, in animal models this vaccine yields high neutralising antibody titres against both RSV A and RSV B.

Thus in a first aspect, provided herein is an mRNA molecule encoding a nanoparticle subunit, the nanoparticle subunit comprising an immunogen and a scaffold joined by a linker, wherein the linker is 9 to 13 amino acids in length, the scaffold comprises a lumazine synthase, and the immunogen comprises a modified RSV F protein comprising a deletion of RSV F protein positions 104-144, a GS peptide linker between RSV F protein positions 103 and 145, and the following mutations: S155C, N183GC, S190F, V207L, S290C, L373R and N428C.

When expressed by a cell, the nanoparticle subunit multimerises via the lumazine synthase scaffold to form a protein nanoparticle, of which the immunogen forms the exposed exterior. In particular instances, the protein nanoparticle comprises 60 self-assembled nanoparticle subunits (i.e., is a 60-mer).

In particular instances, the modified RSV F protein is the modified RSV F protein of SEQ ID NO: 1 or SEQ ID NO: 2, or a variant of SEQ ID NO: 1 or SEQ ID NO: 2, which are described below.

In a second aspect, provided herein is an RSV vaccine (specifically an RSV mRNA vaccine) comprising the mRNA molecule of the first aspect.

In a third aspect, provided herein is the mRNA molecule of the first aspect or the vaccine of the second aspect for use as a therapeutic or prophylactic agent.

In a fourth aspect, provided herein is the mRNA molecule of the first aspect or the vaccine of the second aspect for use in inducing an immune response in a subject. Relatedly, this aspect provides a method of inducing an immune response in a subject, comprising administering to the subject the mRNA molecule of the first aspect or the vaccine of the second aspect. Also relatedly, this aspect provides use of the mRNA molecule of the first aspect or the vaccine of the second aspect in the manufacture of a medicament for use in a method of inducing an immune response in a subject.

The vaccines provided herein find utility in the prevention of RSV infection and diseases associated with RSV infection, and the prevention of serious illness associated with an RSV infection. Thus in a fifth aspect, provided herein is the RSV vaccine of the second aspect, or the mRNA molecule of the first aspect, for use in a method of preventing or attenuating a disease caused by an RSV infection in a subject. As discussed further below, a disease caused by an RSV infection may alternatively be referred to herein as “an RSV infectious disease”.

Relatedly, this aspect provides a method of preventing or attenuating a disease associated with RSV infection in a subject, comprising administering to the subject the RSV vaccine of the second aspect or the mRNA molecule of the first aspect.

This aspect also provides the use of the RSV vaccine of the second aspect, or the mRNA molecule of the first aspect, in the manufacture of a medicament for use in preventing or attenuating a disease caused by an RSV infection.

In this fifth aspect, the disease caused by an RSV infection may be a lower respiratory tract disease (LRTD), such as bronchiolitis, bronchitis or pneumonia.

In a sixth aspect, provided herein is an expression vector comprising an expression cassette encoding an mRNA molecule of the first aspect.

In a seventh aspect, provided herein is a cell comprising the expression vector of the sixth aspect. Such a cell may be referred to as a “host cell”.

In an eighth aspect, provided herein is a method of manufacturing the mRNA molecule of the first aspect, the method comprising expressing the mRNA from the expression vector of the sixth aspect. In some instances, the mRNA is expressed by in vitro transcription.

In a ninth aspect, provided herein is a protein nanoparticle comprising a multimer of the nanoparticle subunit as defined in the first aspect. That is to say, this aspect provides a protein nanoparticle made up of multiple copies of a nanoparticle subunit comprising an immunogen and a scaffold joined by a linker, wherein the linker is 9 to 13 amino acids in length, the scaffold comprises a lumazine synthase, and the immunogen comprises a modified RSV F protein comprising a deletion of RSV F protein positions 104-144, a GS peptide linker between RSV F protein positions 103 and 145, and the following mutations: S155C, N183GC, S190F, V207L, S290C, L373R and N428C. The multimer of the nanoparticle subunit is generally a 60-mer (i.e. a multimer comprising 60 copies of the subunit).

### FIGURE LEGENDS

FIG. 1 shows antigenic site display of DS-CAV1 trimer and DS-CAV-1 presented on alternative nanoparticle (NP) scaffolds. Multiple capture antibodies were assayed, specific for epitopes exclusive to the Pre-Fusion conformation (1G7, AM14 or MPE8), the Post-Fusion conformation (MP43), or that bind to both (palivizumab (Pali) and MP43).

FIG. 2 shows RSV-A neutralizing antibody titres derived from mice immunized with mRNA encoding DS-CAV1 trimer, DS2-2 trimer or with recombinant DS-CAV1 protein trimer.

FIG. 3A shows the vaccination schedule of mice receiving a prime/boost regimen of either recombinant DS-CAV1 trimer (Ds-Cav1 Foldon), or mRNA encoding DS-CAV1 trimer (Ds-Cav1 Foldon) or Ds-Cav1 or DS2-2 nanoparticle subunits fused to either ferritin, beta-annulus (Bann) or lumazine synthase (LuS).

FIG. 3B shows Day 14 RSV-A neutralisation titres from the vaccination regimens described in FIG. 3A

FIG. 3C shows Day 42 RSV-A neutralisation titres from the vaccination regimens described in FIG. 3A.

FIG. 3D shows RSV-B neutralisation titres from the vaccination regimens described in FIG. 3A.

FIG. 3E shows the Pre-Fusion/Post-Fusion antibody ELISA responses for all vaccine regimens at Day 14 (left-hand panel) and Day 42 (right-hand panel).

FIG. 4A shows the vaccination regimen of RSV-exposed mice. Mice were intranasally exposed to RSV at Day 0, prior to receiving vaccination (“boost”) at Day 80.

FIG. 4B shows the RSV-A neutralisation titres from vaccines tested in the regimen set out in FIG. 4A. Mice groups were dosed with either PBS (negative control), RSV-Alive virus, DS-CAV1 trimer (DS-CAV1 soluble), or mRNA encoding DS-CAV1 transmembrane, DS2-2 trimer (DS2 Foldon), DS2-2 LuS (DS2 lumazine synthase) and DS2-2 Ferritin (DS2 ferritin).

FIG. 4C shows RSV-B neutralisation titres for the same vaccine candidates described in FIG. 4B.

FIG. 5 is a bar chart comparing the expression of eGFP-encoding mRNA (in A549 cells) with a constant 3′ UTR region (albumin) and candidate 5′ UTRs (UTR-11, -37, -52, -53-identified in the examples). The black bar represents a vector with a control 5′ UTR region (HSD17B4). The selected 5′ UTRs increase the fluorescence intensity of eGFP in A549 cells. Candidate 5′ UTRs show the ability to increase eGFP expression in A549 cells, compared to a control 5′ UTR (HSD17B4). The data is representative of 3 independent experiments, with 3-4 technical replicates in each experiment.

FIG. 6 is a bar chart comparing the expression of eGFP-encoding mRNA (in HeLa cells) with a constant 3′ UTR region (albumin) and candidate 5′ UTRs (UTR-11, -37, -52, -53). The black bar represents a vector with a control 3′ UTR (albumin) and a control 5′ UTR (HSD17B4). Candidate 5′ UTRs show the ability to increase eGFP expression in HeLa cells, compared to a control 5′ UTR (HSD17B4). The data is representative of 3 independent experiments, with 3-4 technical replicates in each experiment.

FIG. 7 is a bar chart comparing the expression of eGFP mRNA (in A549 cells) with candidate 3′ UTR regions (UTR-3, -4, -36-identified in the examples) and a control 5′ UTR region (HSD17B4). The black bar represents a control 3′ UTR (albumin) and a 5′ UTR (HSD17B4). Candidate 3′ UTRs can increase the expression of eGFP mRNA in A549 cells when compared to a control 3′ UTR. The data is representative of 2 independent experiments, with 3 technical replicates in each experiment.

FIG. 8 is a bar chart comparing the expression of eGFP mRNA (in HeLa cells) with candidate 3′ UTRs (UTR-3, -4, -36) and a control 5′ UTR (HSD17B4). Candidate 3′ UTRs can increase the expression of eGFP mRNA in HeLa cells, when compared to a control 3′ UTR. The black bar represents a control 3′ UTR (albumin) and a control 5′ UTR (HSD17B4). The data is representative of 2 independent experiments, with 3 technical replicates in each experiment.

FIG. 9 is a bar chart comparing the expression of eGFP mRNA (in A549 cells) with combinations of candidate 3′ UTRs (CHIT-1, CS) and 5′ UTRs (GOT1, PRKACB, CHIT1) to a control consisting of a control 3′ UTR (albumin) and a control 5′ UTR (HSD17B4). The black bar represents eGFP mRNA expression in the control. Combining a candidate 3′ UTR with a candidate 5′ UTR can increase eGFP mRNA expression in A549 cells. The data is representative of 3 independent experiments, with 3 technical replicates in each experiment.

FIG. 10 is a bar chart demonstrating the expression of eGFP-encoding modified mRNA (in A549 cells) with a combination of candidate 5′ UTRs (GOT1, PRKACB, CHIT) and 3′ UTRs (CHIT, CS). The black bar represents the expression of eGFP-encoding modified mRNA with control 5′ (HSD17B4) and 3′ UTR (albumin) regions. The combinations of candidate 5′ UTRs and candidate 3′ UTRs increase the expression of eGFP-encoding modified mRNA, when compared to expression with control 5′ and 3′ UTRs. The data is representative of 3 independent experiments, with 3 technical replicates in each experiment.

FIG. 11 shows the antibody expression in A549 cells with scFv-Fc-encoding mRNA with a combination of candidate or control 5′ and 3′ UTRs. The black bar represents the expression of scFv-Fc-encoding mRNA with a control 5′ UTR (HSD17B4) and a control 3′ UTR (Albumin). A combination of PRKACB (5′ UTR)/CHIT (3′ UTR) or CHIT (5′ UTR)/CS (3′ UTR) yields higher expression of scFv-Fc-encoding mRNA than expression with control 5′ and 3′ UTR regions. The data is representative of 3 independent experiments, with 3 technical replicates in each experiment.

FIG. 12 shows the antibody expression in A549 cells with scFv-Fc-encoding modified mRNA (in A549 cells) with a combination of candidate or control 5′ and 3′ UTRs. Modified mRNA contains modified uridine (5-methoxyuridine). The black bar represents the expression of scFv-Fc-encoding mRNA with a control 5′ UTR (HSD17B4) and a control 3′ UTR (Albumin). Combinations of PRKACB (5′ UTR)/CHIT (3′ UTR), PRKACB (5′ UTR)/CS (3′ UTR), CHIT (5′ UTR)/CHIT (3′ UTR) and CHIT (5′ UTR)/CS (3′ UTR) yield higher expression of scFv-Fc-encoding mRNA than expression with control 5′ and 3′ UTR regions. The data is representative of 3 independent experiments, with 3 technical replicates in each experiment.

FIG. 13A shows that mRNAs employing the 5′ UTR CHIT1 and 3′ UTR CS UTR generated the highest levels of eGFP expression among BHK-21 cells compared to two competitor mRNA molecules. mRNA_AZ and mRNA comp A and mRNA comp B all comprise 100% pseudo U. An mRNA_AZ with 0% pseudo U was included for comparative purposes.

FIG. 13B shows that mRNAs employing the 5′ UTR CHIT1 and 3′ UTR CS UTR generated the highest levels of eGFP expression among HEK293 cells compared to two competitor mRNA molecules. mRNA_AZ and mRNA comp A and mRNA comp B all comprise 100% pseudo U. An mRNA_AZ with 0% pseudo was included for comparative purposes.

FIGS. 14A-14B show that the RSV mRNA-VLP vaccine elicited high-titre neutralising antibodies in RSV-experienced non-human primates. Groups of RSV-experienced non-human primates (NHPs) were immunised with a benchmark RSV F protein vaccine (DS-CAV1+adjuvant) or two different RSV mRNA vaccines encoding the prefusion stabilised F protein and the immunogenicity was evaluated by ELISA binding titres (FIG. 14A) and virus neutralisation assays (FIG. 14B).

FIGS. 15A-15D show that the RSV mRNA-VLP vaccine protected infected cotton rats from virus replication in the lungs (FIG. 15B) and nasal tissue (FIG. 15D). Groups of naïve cotton rats were immunised with a benchmark RSV F protein vaccine (DS-CAV1+adjuvant) or two different RSV mRNA vaccines encoding the prefusion stabilised F protein to evaluate vaccine efficacy in a virus challenge model.

### DESCRIPTION

Over the past century or so, vaccines based on attenuated live pathogens, inactivated pathogens and toxoids have transformed public health, and have eliminated or nearly eliminated fearsome diseases such as smallpox, polio and tetanus. However, it has not proven possible to develop effective vaccines to all target infectious diseases using these traditional vaccine technologies. One such target is respiratory syncytial virus (RSV). RSV is an extremely common, seasonal virus which causes respiratory tract infections. In healthy adults and older children RSV infection generally causes mild illness, such as the common cold, but in infants, the elderly and immunocompromised individuals it can cause more severe, lower respiratory tract disease, particularly bronchiolitis in infants and pneumonia in adults.

A formalin-inactivated RSV virus was developed in the 1960s, but this was found to cause antibody-enhanced disease (ADE) in children and bonnet monkeys (Ponnuraj et al., Journal of Infectious Diseases 187(8): 1257-1263, 2003). Since then, new vaccine technologies have been developed, one of which is nanoparticle-based vaccines. One type of nanoparticle of interest is protein nanoparticles, including protein nanocages, which are formed by self-assembly of protein subunits into a symmetrical, multimeric structure. Protein nanocages contain high density, repetitive regions on their surfaces that can be recognised and cross-linked by B cell receptors, driving a greater immune response than is obtained by a basic protein subunit (Curley & Putnam, Frontiers in Bioengineering and Biotechnology 10: 867119, 2022). Another new vaccine technology which has recently found prominence is mRNA vaccines.

Provided herein is an mRNA molecule for use as an mRNA vaccine against RSV. The mRNA encodes a fusion protein comprising an RSV immunogen linked to a scaffold based on lumazine synthase. Lumazine synthase multimerises to form protein nanoparticles, and thus the encoded fusion protein constitutes a protein nanoparticle subunit (referred to herein as simply a “nanoparticle subunit”).

mRNA Molecules

The term “mRNA molecule” is used herein to refer to an RNA (ribonucleic acid) molecule which encodes a polypeptide and can be translated to produce the encoded polypeptide. The polypeptide may be of any length, and though a polypeptide may be naturally occurring or synthetic, in the context of the mRNA molecule provided herein, the polypeptide is a synthetic fusion protein, as described below. The mRNA molecule may be translated to produce the encoded polypeptide in any context, e.g. in vitro, in vivo or ex vivo.

The mRNA molecule may encode multiple polypeptides or only a single polypeptide. Where an mRNA molecule encodes multiple polypeptides this may be in the context of a single open reading frame (ORF), e.g. utilising a 2A skipping sequence to divide them, or in the context of multiple open reading frames. Where an mRNA molecule comprises multiple open reading frames, translation can be initiated from an internal ribosome entry site (IRES). Generally, however, the mRNA molecules provided herein comprise a single open reading frame encoding a single polypeptide (the above-mentioned fusion protein).

The basic components of an mRNA molecule typically include at least one coding region (as discussed above), a 5′ untranslated region (UTR), a 3′ UTR, a 5′ cap and a poly-A tail. The mRNA molecules provided herein may have the structure of naturally-occurring mRNA, or may be distinguished from wild-type mRNA in their functional and/or structural design features.

In some instances, the mRNA molecule is a self-amplifying mRNA (saRNA) molecule. Unlike a traditional mRNA molecule, a saRNA molecule replicates itself after cellular entry. In addition to the basic mRNA components described above, an saRNA molecule comprises a second open reading frame (ORF) encoding an RNA-dependent RNA polymerase (along with its helper proteins), which is expressed within cells and replicates the mRNA molecule. This can allow smaller doses of mRNA to be used compared to vaccines using non-amplifying mRNA molecules, but substantially increases the size of the mRNA molecule to be delivered.

In some instances, the mRNA molecule is a circular RNA (circRNA) molecule. Synthesis of circRNA can be achieved by producing a traditional, linear mRNA molecule and then ligating the 5′ and 3′ ends to produce a circular structure (as described in e.g. Obi & Chen, Methods 196: 85-103, 2021). Due to the lack of termini and inaccessibility to exonucleases, circRNA can be more stable than linear mRNA, and so use of circRNA is another means by which the required mRNA dosage can be reduced.

mRNA Structure

The mRNA molecules provided herein may comprise one or more modified nucleotides or nucleosides. Such a modified nucleotide may be a modified adenosine (A), guanosine (G), uridine (U) or cytidine (C). By “modified” here is meant that the structure of the nucleotide is altered relative to the natural structure. For any given nucleotide, all instances of the nucleotide in the mRNA may be modified, all instances may be unmodified (i.e. have the native structure of the nucleotide), or a certain percentage of the nucleotide in the mRNA may be modified, e.g. about 10, 20, 30, 40, 50, 60, 70, 80 or 90% of the instances of the nucleotide in the mRNA may be modified. Where the mRNA contains a modified nucleotide, the percentage of the nucleotide in the mRNA which may be modified may be e.g. 5 to 20%, 5 to 25%, 5 to 50%, 5 to 60%, 5 to 70%, 5 to 80%, 5 to 90%, 5 to 95%, 10 to 20%, 10 to 25%, 10 to 50%, 10 to 60%, 10 to 70%, 10 to 80%, 10 to 90%, 10 to 95%, 10 to 100%, 20 to 25%, 20 to 50%, 20 to 60%, 20 to 70%, 20 to 80%, 20 to 90%, 20 to 95%, 20 to 100%, 50 to 60%, 50 to 70%, 50 to 80%, 50 to 90%, 50 to 95%, 50 to 100%, 70 to 80%, 70 to 90%, 70 to 95%, 70 to 100%, 80 to 90%, 80 to 95%, 80 to 100%, 90 to 95%, 90 to 100%, or 95 to 100%. A modified nucleotide may comprise a modified nucleobase (modified adenine, guanine, cytosine or uracil), a modified ribose sugar or a modified phosphate group.

Where a proportion of a particular nucleotide (e.g. adenosine) is modified, but not all instances of the nucleotide in the mRNA, the modified nucleotides may be evenly or randomly distributed across the mRNA, or may be localised in particular, chosen sequence regions. For instance, the modified nucleotide may be utilised in the coding region but not the UTRs.

The mRNA molecule may contain modified versions of 1, 2, 3 or all 4 natural nucleosides, e.g. modified adenosine and modified cytidine; modified adenosine and modified uridine; modified adenosine and modified guanosine; modified cytidine and modified uridine; modified cytidine and modified guanosine; modified uridine and modified guanosine; modified adenosine, cytidine, and guanosine; modified adenosine, cytidine and uridine; modified adenosine, guanosine and uridine; or modified cytidine, guanosine and uridine.

Where the mRNA molecule contains one or more modified nucleotides, the total proportion of nucleotides in the molecule which are modified may be anything, up to 100%. For example, the mRNA may comprise about 10, 20, 30, 40, 50, 60, 70, 80 or 90% modified nucleotides. For instance, the mRNA may comprise 1 to 10%, 1 to 20%, 1 to 25%, 1 to 50%, 1 to 60%, 1 to 70%, 1 to 80%, 1 to 90%, 1 to 95%, 1 to 100%, 5 to 10%, 5 to 20%, 5 to 25%, 5 to 50%, 5 to 60%, 5 to 70%, 5 to 80%, 5 to 90%, 5 to 95%, 10 to 20%, 10 to 25%, 10 to 50%, 10 to 60%, 10 to 70%, 10 to 80%, 10 to 90%, 10 to 95%, 10 to 100%, 20 to 25%, 20 to 50%, 20 to 60%, 20 to 70%, 20 to 80%, 20 to 90%, 20 to 95%, 20 to 100%, 50 to 60%, 50 to 70%, 50 to 80%, 50 to 90%, 50 to 95%, 50 to 100%, 70 to 80%, 70 to 90%, 70 to 95%, 70 to 100%, 80 to 90%, 80 to 95%, 80 to 100%, 90 to 95%, 90 to 100%, or 95 to 100%.

In some instances, the mRNA does not contain any modified nucleotides, i.e. it contains only the native adenosine, cytidine, guanosine and uridine structures.

The mRNA molecule provided herein may be a modified mRNA molecule comprising modified internucleoside linkages, e.g. it may comprise modifications to a linking phosphate, to a phosphodiester linkage or to the phosphodiester backbone.

Modified mRNA molecules (i.e. mRNA molecules comprising one or more modified nucleotides and/or modified internucleoside linkages) may be synthesised using modified nucleotides or may be modified post-synthesis.

A “nucleoside” as used herein refers to a compound containing a sugar molecule or a derivative thereof (in the context of RNA, a ribose sugar or derivative thereof) in combination with an organic base (e.g., a purine or pyrimidine) or a derivative thereof (also referred to herein as “nucleobase”). The nucleobases found in the mRNA molecule provided herein are generally the standard RNA nucleobases, i.e. adenine, cytosine, guanine and uracil. A “nucleotide” refers to a nucleoside, including a phosphate group. Modified mRNA molecules may by synthesized by any suitable method, e.g. chemically, enzymatically, or recombinantly, to include one or more modified or non-natural nucleotides or internucleoside linkages.

Where a modified nucleotide comprises a modified ribose moiety, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g. R=an alkyl, cycloalkyl, aryl, aralkyl, heteroaryl or sugar moiety); polyethyleneglycols (PEG); “locked” nucleic acids (LNA) in which the 2′ hydroxyl is connected, e.g. by a methylene bridge, to the 4′ carbon of the same ribose sugar; and amino groups (—O-amino, wherein the amino group can be e.g. an alkylamino, dialkylamino, heterocyclylamino, arylamino, diarylamino, heteroarylamino, diheteroaryl amino, ethylene diamine, polyamino or aminoalkoxy group).

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The ribose moiety may alternatively or further comprise any additional suitable or desired modification at the 2′ OH group or at any other position on the sugar.

Where a modified nucleotide comprises a modified nucleobase, the nucleobase may be modified at any location by any suitable or desired modification, e.g. by addition of an amino group, a thiol group, an alkyl group, or a halo group.

Modified nucleosides which may be used in the mRNA molecules provided herein include: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine; 2-methyladenosine; 2-methylthio-N6-isopentenyladenosine; 2-methylthio-N6-hydroxynorvalyl carbamoyladenosine; N6-(cis-hydroxyisopentenyl)adenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methylthio-N6-isopentenyladenosine; 7-deaza-adenosine; N1-methyladenosine; N6-cis-hydroxy-isopentenyladenosine; α-thio-adenosine; 2-aminoadenosine; 2-(aminopropyl)adenosine; 2-propyladenosine; 2′-amino-2′-deoxyadenosine; 2′-azido-2′-deoxyadenosine; 8-aminoadenosine; 8-hydroxyadenosine; 8-thioadenosine; 8-azidoadenosine; 8-azaadenosine; 7-deaza-8-aza-adenosine; 7-methyladenosine; 1-deazaadenosine; 2′-fluoro-N6-benzoyl-deoxyadenosine; 2′-O-methyl-2-amino-adenosine; 2′-O-methyl-N6-benzoyl-deoxyadenosine; 2-ethynyladenosine; 2′-O-trifluoromethyladenosine; 2-azidoadenosine; 2′-ethynyladenosine; 2-bromoadenosine; 2-trifluoromethyladenosine; 2-chloroadenosine; 2′-deoxy-2,2′-difluoroadenosine; 2′-deoxy-2′-mercaptoadenosine; 2′-deoxy-2′-aminoadenosine; 2′-deoxy-2′-azidoadenosine; 2′-deoxy-2′-bromoadenosine; 2′-deoxy-2′-chloroadenosine; 2′-deoxy-2′-fluoroadenosine; 2′-deoxy-2′-iodoadenosine; 2-fluoroadenosine; 2-iodoadenosine; 2-mercaptoadenosine; 2-methoxyadenosine; 2-methylthioadenosine; 3-deaza-3-bromoadenosine; 3-deaza-3-chloroadenosine; 3-deaza-3-fluoroadenosine; 3-deaza-3-iodoadenosine; 3-deazaadenosine; 4′-azidoadenosine; 8-bromoadenosine; 8-trifluoromethyladenosine; 9-deazaadenosine; 2-thiocytidine; 3-methylcytidine; 5-hydroxymethylcytidine; 5-methylcytidine; N4-acetylcytidine; 2′-O-methylcytidine; 5-formyl-2′-O-methylcytidine; lysidine; N4,2′-O-dimethylcytidine; N4-acetyl-2′-O-methylcytidine; N4-methylcytidine; N4,N4-dimethyl-2′-O-methylcytidine; 4-methylcytidine; 5-azacytidine; pseudoisocytidine; α-thio-cytidine; 2′-amino-2′-deoxycytidine; 2′-azido-2′-deoxycytidine; 3-deaza-5-azacytidine; 5-propynylcytidine; 5-trifluoromethylcytidine; 5-bromocytidine; 5-iodocytidine; 6-azacytidine; pseudoisocytidine; 2′-O-methyl-5-methyl-cytidine; 2-thio-5-methyl-cytidine; 5-methylzebularine; zebularine; (E)-5-(2-bromovinyl)cytidine; N4-benzoyl-2′-fluoro-2′-deoxycytidine; N4-acetyl-2′-fluoro-2′-deoxycytidine; 2′-O-methyl-N4-acetylcytidine; N4-benzoyl-2′-O-methylcytidine; 2′-ethynylcytidine; 5-trifluoromethyl-2′-deoxycytidine; 2′-deoxy-2′,2′-difluorocytidine; 2′-deoxy-2′-mercaptocytidine; 5-bromo-2′-deoxycytidine; 2′-chloro-2′-deoxycytidine; 2′-deoxy-2′-fluorocytidine; 5-iodo-2′-deoxycytidine; 5-(1-propynyl)-2′-O-methylcytidine; 3′-C-ethynylcytidine; 4′-azidocytidine; 5-aminoallylcytidine; cyanocytidine; 5-ethynylcytidine; 5-methoxycytidine; N4-aminocytidine; N4-benzoylcytidine; 7-methylguanosine; N2,2′-O-dimethylguanosine; N2-methylguanosine; wyosine; 1,2′-O-dimethylguanosine; 1-methylguanosine; 2′-O-methylguanosine; 7-aminomethyl-7-deazaguanosine; 7-cyano-7-deazaguanosine; archaeosine; methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thioguanosine; 7-deazaguanosine; 8-oxoguanosine; α-thio-guanosine; 2′-amino-2′-deoxyguanosine; 2′-azido-2′-deoxyguanosine; 6-O-methylguanosine; 8-aminoguanosine; 8-hydroxyguanosine; 8-thioguanosine; 8-azaguanosine; 7-methyl-6-thioguanosine; 6-thio-7-methylguanosine; 7-deaza-8-azaguanosine; 7-methyl-8-oxoguanosine; N2-isobutyryl-2′-O-methylguanosine; 2′-deoxy-2′,2′-difluoroguanosine; 2′-deoxy-2′-chloroguanosine; 2′-deoxy-2′-fluoroguanosine; 8-bromoguanosine; 9-deazaguanosine; 1-methylinosine; inosine; 1,2′-O-dimethylinosine; 2′-O-methylinosine; 7-methylinosine; epoxyqueuosine; galactosyl-queuosine; mannosyl-queuosine; queuosine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; dihydrouridine; pseudouridine; 3-(3-amino-3-carboxypropyl)uridine; 1-methyl-3-(3-amino-3-carboxypropyl)pseudouridine; 1-methylpseudouridine; 2′-O-methylpseudouridine; 2-thio-2′-O-methyluridine; N3-methyl-2′-O-methyluridine; 3-methylpseudouridine; 4-thiouridine; 5-carboxyhydroxymethyluridine; 5-methyl-2′-O-methyluridine; 5,6-dihydrouridine; 5-aminomethyl-2-thiouridine; 5-carbamoylmethyl-2′-O-methyluridine; 5-carbamoylmethyluridine; 5-carboxymethylaminomethyl-2′-O-methyluridine; 5-carboxymethylaminomethyl-2-thiouridine; 5-carboxymethylaminomethyluridine; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-methyldihydrouridine; uridine-5-oxyacetic acid; methyluridine-5-oxyacetic acid; 5-(isopentenylaminomethyl)uridine; 5-propynyluridine; α-thio-uridine; 2′-deoxyuridine; 2′-deoxy-2′-fluorouridine; 2′-amino-2′-deoxyuridine; 2′-azido-2′-deoxyuridine; 4-thiopseudouridine; 5-(aminopropyl)uridine; 5-methyl-4-thiouridine; 5-(trifluoromethyl)uridine; 5-(3-aminopropyl)uridine; 5-aminoallyluridine; 5-bromouridine; 5-iodouridine; 5-chlorouridine; 5-fluorouridine; 6-azauridine; 3-deazauridine; 2-thio-6-azauridine and 2-thiopseudouridine.

Where the mRNA molecule comprises a modified version of a particular nucleotide, it may contain one or more, e.g. 2, 3 or 4 different modified versions of that nucleotide.

In particular instances, the mRNA molecule comprises modified uridine nucleotides, in particular pseudouridine or modified pseudouridine nucleotides. Pseudouridine is an isomer of the natural uridine nucleoside in which uracil is attached to ribose via a carbon-carbon bond instead of a nitrogen-carbon glycosidic bond. Examples of modified pseudouridine nucleosides include those set out above. The mRNA molecule may comprise pseudouridine and/or one or more species of modified pseudouridine. The mRNA molecule may comprise exclusively pseudouridine or modified pseudouridine nucleosides (i.e. all uridine residues may be replaced with pseudouridine or modified pseudouridine residues), or only a proportion of uridine residues may be replaced with pseudouridine or modified pseudouridine residues.

In particular instances, the mRNA molecule comprises the modified pseudouridine N1-methylpseudouridine (also referred to as 1-methylpseudouridine). The structure of N1-methylpseudouridine is set out below in Formula I.

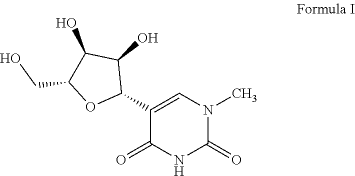 [SMILES: Cn1cc([C@@H]2O[C@H](CO)[C@@H](O)[C@H]2O)c(=O)[nH]c1=O]

In particular instances, the mRNA molecule contains exclusively N1-methylpseudouridine. That is to say, in these instances all uridine residues are replaced with N1-pseudouridine. In other instances the mRNA molecule comprises at least 10, 20, 30, 40, 50, 60, 70, 80 or 90% N1-methylpseudouridine (i.e. at least 10, 20, 30, 40, 50, 60, 70, 80 or 90% of uridine residues in the mRNA molecule may be replaced with N1-methylpseudouridine).

Where the internucleoside linkages are modified, the phosphate groups of the backbone can be modified by replacing one or more of the oxygen atoms with a different substituent. Further, the nucleotides can be modified by the full replacement of an unmodified phosphate moiety with a modified phosphate as described herein. Examples of modified phosphate groups which may be used in the mRNA molecules provided herein include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) or carbon (bridged methylene-phosphonates).

Naturally-occurring mRNA comprises a 5′ cap structure. The mRNA molecule provided herein may similarly comprise a 5′ cap, which is linked to the 5′ terminal nucleotide of the mRNA molecule. The cap may have any suitable structure. A 5′ cap may typically be formed by a modified nucleotide, particularly by a derivative of a guanine nucleotide. Generally, the 5′ cap is linked to the 5′ terminus via a 5′-5′-triphosphate linkage.

The cap may have a Cap0 structure, i.e. an N7-methylguanosine connected to the 5′ nucleotide of the mRNA molecule through a 5′ to 5′ triphosphate linkage. A Cap0 structure may be referred to as an m7G or m7GpppN cap (where N indicates any nucleotide). The Cap0 structure is set out below in Formula II (showing a guanosine residue as an example of the first residue of the mRNA, though this may in practice be any nucleotide):

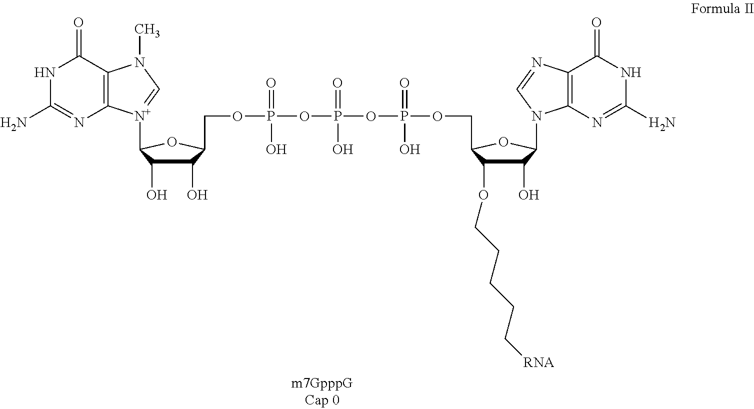

Alternatively, the mRNA cap may have a Cap1 structure. The Cap1 structure differs to the Cap0 structure by the addition of a methyl group at the 2′O position on the first residue of the mRNA. The Cap1 structure may be referred to as an m7GpppNm cap (where Nm refers to any 2′-O-methylated nucleotide). This structure may alternatively be presented as m7Gppp (2′Om)N. A Cap1 structure is shown in Formula III below (again showing guanosine as an exemplary first residue):

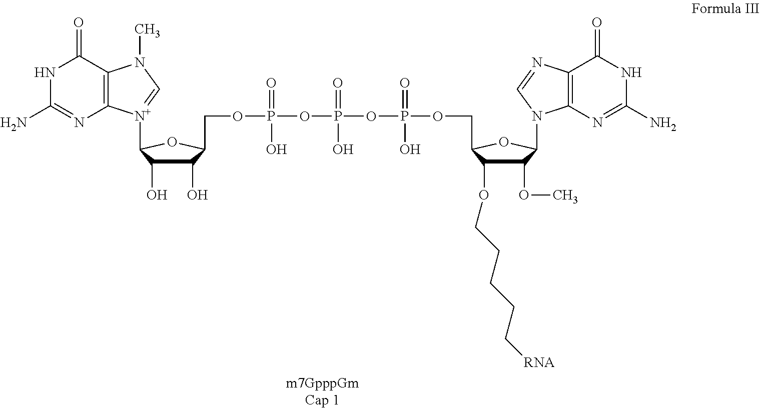

In particular instances, the mRNA molecule provided herein comprises a 5′ cap having a Cap1 structure. The Cap1 structure may in particular have the structure m7GpppAm (i.e. wherein the first nucleotide in the mRNA molecule is adenosine).

In other instances, the mRNA molecule provided herein comprises a 5′ cap having a modified Cap1 structure. A modified Cap1 structure is defined herein as a Cap1 structure (i.e. comprising the m7GpppNm structure) having an additional structural modification to the methylguanosine cap itself, the 5′ nucleotide of the mRNA molecule or the triphosphate linker between them. For example, the 5′ cap may have a Cap1 structure in which the N7-methylguanosine cap comprises an additional methyl group at the 3′O position. Such a modified Cap1 may be referred to as an m7(3′Om)GpppNm cap. In particular instances, the mRNA molecule comprises a modified cap with the structure m7(3′Om)GpppAm (i.e. wherein the first nucleotide residue of the mRNA molecule is adenosine). An exemplary structure of such a cap (showing by way of example a guanosine residue at position 2 of the mRNA molecule) is shown in Formula IV below:

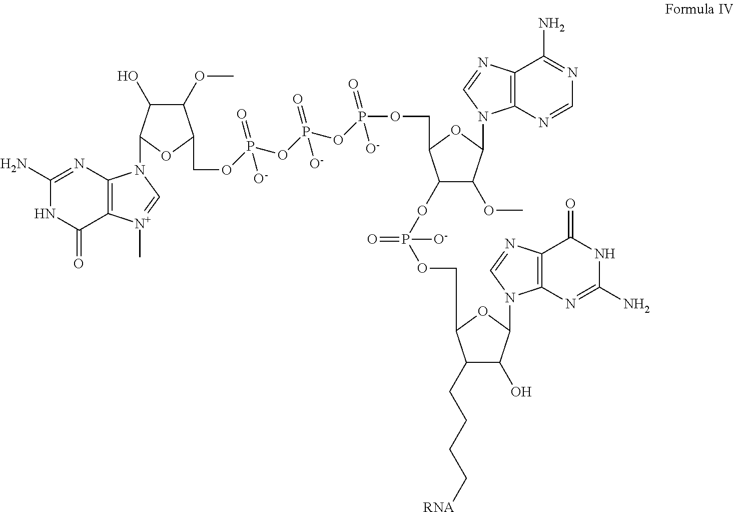

Another example of a modified Cap1 structure is a cap having the structure m7(3′Om)Gpppm6(2′Om)A. Such a cap is based on the m7(3′Om)GpppAm cap shown in the figure above above, further comprising an additional methyl group at position 6 of the adenosine residue at the 5′ terminus of the mRNA molecule. An exemplary structure of such a cap (showing by way of example a guanosine residue at position 2 of the mRNA molecule) is shown in Formula V below:

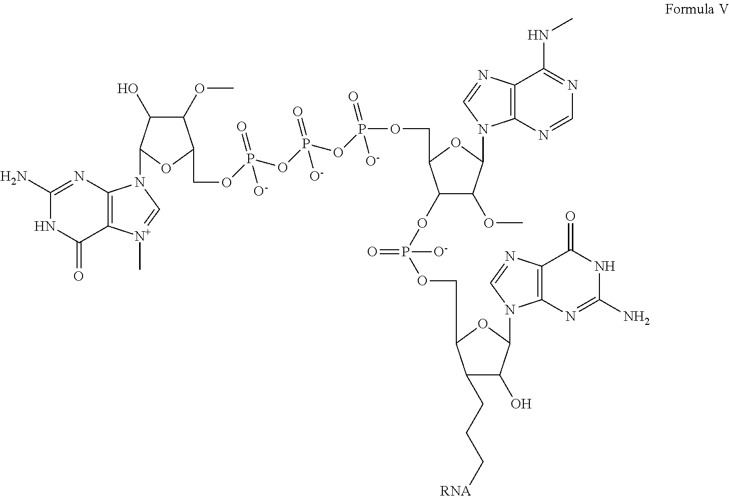

Such caps can be applied to mRNA molecules by any suitable process, e.g. chemically or enzymatically. For example, a Cap1 structure can be applied to mRNA using the CleanCap® Reagent AG (TriLink, CA, USA). Modified Cap1 structures of Formula IV and Formula V can be applied to mRNA using the CleanCap® Reagent AG (3′OMe) and the CleanCap® Reagent M6, respectively.

Other suitable 5′ cap structures include e.g. a Cap2 structure or an ARCA cap analogue. Where the mRNA molecule is a circular RNA molecule, no cap is present.

### UTRs and Other Sequence Elements

As noted above, mRNA molecules naturally comprise a 5′ UTR and a 3′ UTR. The mRNA molecule provided herein may similarly comprise a 5′ UTR and/or a 3′ UTR, generally both a 5′ UTR and a 3′ UTR. The term “3′ UTR” refers to a part of the mRNA molecule which is located 3′ (i.e. “downstream”) of the coding region and which is not translated into protein. Typically, a 3′ UTR is the part of an mRNA which is located between the protein coding region (coding region or coding sequence (CDS)) and the poly(A) sequence of the mRNA. Where an mRNA contains multiple CDSs, the 3′ UTR is located 3′ to the final (i.e. 3′) CDS. A 3′ UTR may commence immediately 3′ to the stop codon of the CDS (or final CDS), or an intervening nucleotide or nucleotide sequence may be present. The 3′ UTR may be of any suitable length, e.g. at least 20, 30, 40, 50 or 60 nucleotides long, e.g. 30 to 100, 40 to 90, 50 to 80 or 60 to 70 nucleotides long.

The term “5′ UTR” refers to a part of the mRNA molecule which is located 5′ (i.e. upstream) of the coding region and which is not translated into protein. Typically, for an mRNA transcribed from DNA, the 5-UTR starts with the transcriptional start site and ends one nucleotide before the start codon of the coding region (or where multiple coding regions are present, the first (5′) coding region). The 5-UTR may comprise elements for controlling gene expression, also called regulatory elements. Such regulatory elements may be, for example, ribosomal binding sites. The 5′ nucleotide of the mRNA molecule is located within the 5′ UTR, and as noted above this is capped. The 5′ UTR may be of any suitable length, e.g. at least 10, 15, 20, 25 or 30 nucleotides long, e.g. 10 to 50, 20 to 40 or 30 to 35 nucleotides long.

Some 5′ UTRs comprise a 5′ terminal oligopyrimidine tract (TOP). This is typically a stretch of pyrimidine nucleotides located in the 5′ UTR. The sequence generally starts with a cytidine, which usually corresponds to the transcriptional start site, and is followed by a stretch of e.g. about 3 to 30 pyrimidine nucleotides. For example, a TOP may comprise at least 3, 5, 10, 15, 20, 25 or 30 pyrimidine nucleotides. The pyrimidine stretch and thus the 5′ TOP ends one nucleotide 5′ to the first purine nucleotide located downstream of the TOP.

The mRNA molecule may comprise at its 3′ end a polyadenosine (polyA) tail. The polyA tail is located 3′ to the 3′ UTR. Alternatively viewed, the polyA tail may be seen as part of the 3′ UTR. The polyA tail may comprise about 10 to 200 adenosine residues, generally about 40 to 100 adenosine residues, e.g. about 40, 50, 60, 70 or 80 adenosine residues, e.g. 40 to 90, 40 to 80, 40 to 70, 40 to 60, 40 to 50, 50 to 100, 50 to 90, 50 to 80, 50 to 70, 50 to 60, 60 to 100, 60 to 90, 60 to 80, 60 to 70, 70 to 100, 70 to 90, 70 to 80, 75 to 85, 75 to 80 or 80 to 100 adenosine residues.

Any suitable combination of UTRs may be used in the mRNA molecule provided herein. The 5′ and 3′ UTRs may originate or be derived from a gene or may be synthetic. The 5′ and 3′ UTRs may have the same source or different sources, e.g. the 5′ UTR may be synthetic and the 3′ UTR may originate or be derived from a gene, or vice versa. A UTR originating from a gene refers to a UTR which is identical to a UTR found in nature, in the context of a gene. A UTR derived from a gene refers to a UTR which is based on a UTR found in nature, but which is modified relative to that native (parent UTR). A UTR derived from a gene may have e.g. at least 70, 75, 80, 85, 90 or 95% sequence identity to the parent UTR, and/or may comprise additional sequence elements 5′ and/or 3′ to the parent UTR.

Where a UTR originates or is derived from a gene, that gene is a eukaryotic gene, generally a mammalian gene, in particular a human gene.

Examples of such 5′ UTR sequences which can be used in the mRNA molecule provided herein include those set forth in SEQ ID NOs: 16 and 20 to 24. Examples of such 3′ UTR sequences which can be used in the mRNA molecule provided herein include those set forth in SEQ ID NOs: 15 and 25-28.

The 5′ UTR may thus comprise or consist of a nucleotide sequence as set forth in any one of SEQ ID NOs: 16 and 20 to 23, or a variant of any one of SEQ ID NOs: 16 and 20 to 23 having at least 70, 75, 80, 85, 90 or 95% sequence identity thereto. In particular instances the 5′ UTR comprises or consists of the nucleotide sequence of SEQ ID NO: 15, or a variant thereof having at least 90 or 95% sequence identity thereto. An extended UTR comprising SEQ ID NO: 15 with additional sequence elements at both ends is set forth in SEQ ID NO: 43. In particular instances the 5′ UTR comprises or consists of the nucleotide sequence of SEQ ID NO: 43, or a variant thereof having at least 90 or 95% sequence identity thereto.

The 3′ UTR sequence may thus comprise or consist of a nucleotide sequence as set forth in any one of SEQ ID NOs: 16 and 24-26, or a variant of any one of SEQ ID NOs: 15 and 24 to 26 having at least 70, 75, 80, 85, 90 or 95% sequence identity thereto. In a particular instance the 3′ UTR comprises or consists of the nucleotide sequence of SEQ ID NO: 16, or a variant thereof having at least 90 or 95% sequence identity thereto. An extended UTR comprising SEQ ID NO: 16 with additional sequence elements at both ends is set forth in SEQ ID NO: 44. In particular instances the 3′ UTR comprises or consists of the nucleotide sequence of SEQ ID NO: 44, or a variant thereof having at least 90 or 95% sequence identity thereto.

The 5′ and 3′ UTR sequences set forth above may be used in combination. Thus the mRNA molecule may comprise: (i) a 5′ UTR comprising or consisting of nucleotide sequence as set forth in any one of SEQ ID NOs: 15 and 20 to 23, or a variant of any one of SEQ ID NOs: 25 and 20 to 23 having at least 70, 75, 80, 85, 90 or 95% sequence identity thereto; and (ii) a 3′ UTR comprising or consisting of a nucleotide sequence as set forth in any one of SEQ ID NOs: 16 and 24-26, or a variant of any one of SEQ ID NOs: 16 and 24 to 26 having at least 70, 75, 80, 85, 90 or 95% sequence identity thereto.

As shown in the examples below, particularly high levels of protein expression are obtained from mRNA molecules comprising the 5′ UTR of SEQ ID NO: 15 (derived from the human CHIT1 5′ UTR) and a 3′ UTR comprising SEQ ID NO: 16 (derived from the human CS 3′ UTR). Thus in a particular instance, the mRNA molecule comprises: (i) a 5′ UTR comprising or consisting of the nucleotide sequence set forth in SEQ ID NO: 15, or a variant thereof having at least 90 or 95% identity to SEQ ID NO: 15; and (ii) a 3′ UTR comprising or consisting of the nucleotide sequence set forth in SEQ ID NO: 16, or a variant thereof having at least 90 or 95% identity to SEQ ID NO: 16.

As shown below, particularly high levels of protein expression are obtained when the 5′ UTR comprising SEQ ID NO: 15 is the 5′ UTR of SEQ ID NO: 43, and the 3′ UTR comprising SEQ ID NO: 16 is the 3′ UTR of SEQ ID NO: 44. Thus in a particular instance, the mRNA molecule comprises: (i) a 5′ UTR comprising or consisting of the nucleotide sequence set forth in SEQ ID NO: 43, or a variant thereof having at least 90 or 95% identity to SEQ ID NO: 43; and (ii) a 3′ UTR comprising or consisting of the nucleotide sequence set forth in SEQ ID NO: 44, or a variant thereof having at least 90 or 95% identity to SEQ ID NO: 44.

Where variants of the specified UTRs are used, the variants are generally at least functionally equivalent to the specified UTRs. By “functionally equivalent” in the context of UTR sequences is meant that the variant UTRs cause an equivalent (or similar) level of protein expression to the specified UTR sequences. A “similar” level of protein expression may mean that the variant UTR causes a protein expression level which is as high as, or at least 70, 75, 80, 85, 90 or 95% as high as, the specified, unmodified UTR sequence. That the variants are “at least functionally equivalent” to the specified sequences means that the variant sequences may be superior to the specified, unmodified UTR sequence, i.e. they may cause a higher level of protein expression than the specified, unmodified UTR sequence.

### Nanoparticle Subunits

The mRNA molecule provided herein encodes a fusion protein, which when expressed forms a subunit of a protein nanoparticle. That is to say, the mRNA molecule comprises a nucleotide sequence encoding such a fusion protein, or “nanoparticle subunit”. The nanoparticle subunit comprises an immunogen and a scaffold, joined by a linker. When the mRNA molecule is administered to a subject, the nanoparticle subunit is expressed by cells of the subject, causing an immune response against the immunogen.

In some instances, the nanoparticle subunit consists of the immunogen, scaffold and linker. In other instances, the nanoparticle subunit comprises one or more additional elements, e.g. a signal peptide (though in some instances a signal peptide may be included as part of the immunogen).

The components of the nanoparticle subunit may be arranged in either order, that is to say the immunogen may be located at the N-terminus or the C-terminus. Thus, in some instances, the nanoparticle subunit comprises (or consists of) from N-terminus to C-terminus, the immunogen, the linker and the scaffold. In other instances, the nanoparticle subunit comprises (or consists of) from N-terminus to C-terminus, the scaffold, the linker and the immunogen. As noted above, the immunogen and scaffold are joined by the linker, and thus the linker is located in between them. Generally, the immunogen and the scaffold are directly adjacent to the linker, i.e. no additional sequence elements are present between the linker and the immunogen or the scaffold.

### Protein Nanoparticle

Once expressed, multiple copies of the nanoparticle subunit assemble into a protein nanoparticle. A protein nanoparticle is a nanoparticle formed by multimerisation of one or more proteins, such as the nanoparticle subunits disclosed herein.

A protein nanoparticle may be a homomultimer (i.e. may be formed of multiple copies of the same subunit protein) or a heteromultimer (i.e. comprising at least two different subunit proteins). Generally the protein nanoparticle formed by the subunit encoded by the mRNA molecule is a homomultimer, formed from multimerization of the encoded subunit. The encoded subunit multimerises via the scaffold section of the protein, discussed further below.

The nanoparticle formed by the subunit encoded by the mRNA molecule may have a diameter of about 1-100 nm, e.g. 1-80, 1-60, 1-50 1-40, 1-35, 1-30, 1-25, 1-20, 5-100, 5-80, 5-60, 5-50, 5-40, 5-35, 5-30, 5-25, 5-20, 10-100, 10-80, 10-60, 10-50, 10-40, 10-35, 10-30, 10-25 or 10-20 nm. In general, a nanoparticle may have any shape, e.g. it may be a sphere (or approximately a sphere) or an icosahedron. Where the shape does not have a single, uniform diameter, the term “diameter” as used here refers to the longest internal dimension of the nanoparticle. The diameter is measured between the external surfaces of the nanoparticle.

The protein nanoparticle formed by the subunit encoded by the mRNA molecule may comprise e.g. at least 5, 10, 20, 30, 40, 50, 60 or more subunits. In particular instances, the protein nanoparticle contains 60 subunits (i.e. it may be a 60-mer), in particular it may be a homomeric nanoparticle which contains 60 of the subunits encoded by the mRNA molecule. In some instances, the protein nanoparticle may be a homomeric 60-mer.

The protein nanoparticle formed by the subunit encoded by the mRNA molecule may have a molecular weight in the range 650 kDa to 15 mDa, e.g. 650 kDa to 12, 10, 9, 8, 7, 6 or 5 mDa, e.g. 1-15, 1-12, 1-10, 1-9, 1-8, 1-7, 1-6, 1-5, 2-15, 2-12, 2-10, 2-9, 2-8, 2-7, 2-6, 2-5, 3-15, 3-12, 3-10, 3-9, 3-8, 3-7, 3-6, 3-5, 4-15, 4-12, 4-10, 4-9, 4-8, 4-7, 4-6 or 4-5 mDa.

### Scaffold

As noted above, the scaffold essentially functions as a multimerization domain for the encoded nanoparticle subunit, via which the individual subunits interact to form a nanoparticle.

The scaffold is or comprises a lumazine synthase protein. Lumazine synthase is an enzyme found across all domains of life which catalyses the penultimate step in the biosynthesis of riboflavin (vitamin B2). All lumazine synthase proteins multimerise to form complexes of various sizes. Notably, some eubacterial species, and plants, have lumazine synthases which form a 60-meric icosahedral capsid. The structure of such capsids is discussed in Ladenstein & Morgunova, Biotechnology Reports 27: e00494, 2020. Lumazine synthase capsids can be described as protein cage nanoparticles. The capsids are homomeric 60-mers, containing 60 identical lumazine synthase subunits. The 60-meric structures may be considered dodecamers of pentamers, with particularly strong interactions formed between the subunits within each pentamer, and weaker interactions formed between the pentamers which assemble to form the capsid.

In 60-meric lumazine synthase capsids, both the N- and C-terminus of each subunit is exposed on the outer surface. Lumazine synthase capsids can thus be used as nanoparticles for antigen display, by fusing a target antigen to either the N-terminus or C-terminus of the lumazine synthase enzyme.

The lumazine synthase used in the scaffold in the fusion protein described herein may be a lumazine synthase which multimerises to form a 60-mer, i.e. a lumazine synthase which multimerises to form a 60-meric icosahedral capsid, or a 60-meric nanoparticle. The lumazine synthase may originate or be derived from any species which produces such an enzyme, for instance a plant (e.g. spinach (Spinacia oleracea), tobacco (e.g. Nicotiana tabacum or Nicotiana rustica) or Arabidopsis thaliana) or a eubacterium.

For instance, the lumazine synthase may originate or be derived from any eubacterium which produces a 60-meric version of the enzyme. Suitable eubacteria include those of the family Bacillaceae, e.g. Bacillus subtilis, those of the family Enterobacteriaceae, e.g. Escherichia coli, and those of the family Aquificaceae, e.g. Aquifex aeolicus.

As set out above in respect of UTRs, a lumazine synthase originating from a particular species has the native sequence of the enzyme from that species. A lumazine synthase derived from a species is a modified version of the native enzyme. Where a modified version of a naturally occurring lumazine synthase is used, the modified version retains the ability to form multimeric complexes, and at least one of the N- and C-termini remain exposed on the outer surface of the capsid so that the immunogen can be fused to it and displayed on the capsid surface.

A modified version of a lumazine synthase which natively forms a 60-meric structure may retain the ability to form a 60-meric structure, or may form a different structure with more or fewer subunits than 60. Modified bacterial lumazine synthases are known which form 180-meric or 360-meric nanoparticles (Ladenstein & Morgunova, supra), and such modified lumazine synthases may be used in the fusion proteins encoded by the mRNA molecules provided herein.

Alternatively, the lumazine synthase used herein may natively form smaller multimers, comprising e.g. 5 or 10 subunits. Modified versions of such lumazine synthases may be used, which may form larger multimers than the native enzyme.

Where the lumazine synthase used is a variant of a native enzyme, the enzyme may be inactive. Since the enzyme is merely used as a scaffold, it is unimportant whether it retains its synthase activity.

In particular instances the lumazine synthase is the A. aeolicus lumazine synthase, or a derivative thereof. The A. aeolicus lumazine synthase has the amino acid sequence set out in SEQ ID NO: 3 (UniProt entry 066529). Thus the scaffold used in the nanoparticle subunit herein may comprise or consist of the amino acid sequence set forth in SEQ ID NO: 3, or a variant thereof having at least 70, 75, 80, 85, 90 or 95% identity to SEQ ID NO: 3. In particular instances, the scaffold comprises or consists of the amino acid sequence set forth in SEQ ID NO: 3.

Where a variant of SEQ ID NO: 3 is used, the variant retains the ability to form multimeric protein nanoparticles. In particular, the variant may retain the ability to form the 60-meric complexes discussed above. Assembly of A. aeolicus lumazine synthase pentamers into icosahedral capsids has been found to rely on 8 amino acid residues in each subunit, which form hydrophobic interactions (L121 and I125), electrostatic interactions (E5, R21, D36, R40 and E145), and a hydrogen bond (H41), between neighbouring pentamers. Disruption of these interactions (particularly the hydrogen bond) by point mutation has been found to prevent dodecamerisation of the pentameric capsid building blocks, such that the enzyme forms only a pentameric structure (Hickman et al., Proteins 83: 1733-1741, 2015). To avoid loss of the ability to form 60-meric or other larger structures, variants of the enzyme of SEQ ID NO: 3 may be unmodified at these locations, or contain conservative substitutions at these locations which do not disrupt the intersubunit interactions.

### Linker

The present inventors have found that an enhanced immune response is obtained when an immunogen is fused to the lumazine synthase via a linker, rather than directly. Without being bound by theory, it is postulated that when no linker is used, access to important neutralising epitopes is hindered by the proximity of the immunogen to the lumazine synthase multimer.

The linker used is long enough that the immunogen is sufficiently distanced from the lumazine synthase multimer that its epitopes are accessible to antibodies. For instance, the linker may be at least 5, 6, 7, 8, 9 or 10 amino acids long. The linker may have a maximum length of 20, 18, 16, 14 or 12 amino acids. The present inventors have found that for the immunogen of SEQ ID NO: 1 or 2 (further discussed below), the linker is optimally about 9 to 13 amino acids long, e.g. 10 to 12 amino acids long. In particular instances the linker is 11 amino acids long.

The sequence of the linker is not particularly limited, so long as it's long enough to distance the scaffold from the immunogen. Various linker sequences for joining fusion protein constituents are known in the art and can be used in the present fusion protein, including glycine-serine linkers (i.e. linkers consisting of glycine and serine residues), e.g. [G]n, [S]n, [A]n, [GS]n, [GGS]n, [GGGS]n (SEQ ID NO: 27), [GGGGS]n (SEQ ID NO: 28), [GGSG]n (SEQ ID NO: 29), [GSGG]n (SEQ ID NO: 30), [SGGG]n (SEQ ID NO: 31), [SSGG]n (SEQ ID NO: 32), [SSSG]n (SEQ ID NO: 33), [GG]n, [GGG]n, [SA]n, [SAGS]n (SEQ ID NO: 34) or [GGGSG]n (SEQ ID NO: 35), wherein ‘n’ is between 1 and 20 (a suitable value of ‘n’ is dependent on the length of the repeating unit—a suitable value for ‘n’ can be selected to yield a linker of a length falling within the ranges set out above).

For example, suitable linker sequences include TGGGGSGGGGS (SEQ ID NO: 36), GGGGSGGGGS (SEQ ID NO: 37) and SGGSSGSSGGS (SEQ ID NO: 4).

In particular instances, the linker comprises or consists of the amino acid sequence set out in SEQ ID NO: 4, or a variant thereof comprising up to 3 amino acid insertions, deletions or substitutions relative to SEQ ID NO: 4. For instance, a variant of SEQ ID NO: 4 may contain up to 2 amino acid insertions, deletions or substitutions relative to SEQ ID NO: 4, or a single amino acid insertion, deletion or substitution relative to SEQ ID NO: 4.

In particular instances, the linker comprises or consists of the amino acid sequence set out in SEQ ID NO: 4, or a variant thereof comprising up to 3 amino acid substitutions relative to SEQ ID NO: 4, i.e. comprising 1, 2 or 3 amino acid substitutions relative to SEQ ID NO: 4. Such a linker may be an 11 amino acid linker, i.e. consisting of SEQ ID NO: 4 or a substituted variant thereof.

### Linker-Scaffold

The combination of the linker and the scaffold is referred to herein as the “linker-scaffold”. The linker-scaffold may comprise any scaffold and any linker sequence, as set out above, in either orientation (i.e. the linker or the scaffold may be at the N-terminus, and the other at the C-terminus).

In particular instances, the linker scaffold comprises or consists of the amino acid sequence set forth in SEQ ID NO: 5, or a variant thereof having at least 70, 75, 80, 85, 90 or 95% identity to SEQ ID NO: 5. SEQ ID NO: 5 comprises the scaffold of SEQ ID NO: 3 with the linker of SEQ ID NO: 4 at its N-terminus.

### Immunogen

The immunogen comprises a modified RSV F protein, against which it is desired to generate an immune response to yield immunity to RSV The immunogen used herein is an antigen, i.e. the modified RSV F protein will generate an antibody response when expressed in a subject (at least an antibody response against the RSV F protein).

In some instances the immunogen may comprise multiple (i.e. two or more) proteins or protein fragments which are themselves fused together. That is to say, the immunogen may comprise the modified RSV F protein (described further below) and at least one additional protein or protein fragment. Generally, the immunogen is (i.e. consists of) the modified RSV F protein. The RSV F protein is known to be a primary target for neutralising antibodies during RSV infection. The modified RSV F protein may be derived from any RSV strain, e.g. it may be from an RSV A strain (such as subtype A1 or A2) or an RSV B strain (such as B1 or B2).

The modified RSV F protein is stabilised in the pre-fusion conformation (i.e. a pre-F-stabilised F protein). It comprises both an F1 polypeptide and an F2 polypeptide in a single chain, with a linker between them. Specifically, the modified RSV F protein comprises a modified RSV F protein comprising a deletion of RSV F protein positions 104-144 and a GS peptide linker between RSV F protein positions 103 and 145, and the following mutations: S155C, N183GC, S190F, V207L, S290C, L373R and N428C. Thus the C-terminus of an F2 polypeptide is joined to the N-terminus of an F1 polypeptide by a GS linker.

The amino acid numbering as set out above is based on a full-length wild-type RSV F protein. This may by the wild-type RSV F protein upon which the modified F protein is based, or any other wild-type RSV F protein. A full-length F protein is the primary F protein sequence prior to any post-translational modification or processing, including the signal sequence. In particular instances, the amino acid position numbering is based on the exemplary RSV F protein of SEQ ID NO: 19. This full-length RSV F protein is from RSV A strain A2 and has the UniProt accession number P03420 (sequence version 1).

In SEQ ID NO: 19, amino acids 1-25 constitute the signal sequence (SEQ ID NO: 41), and amino acids 26-574 constitute the F0 chain (SEQ ID NO: 38). Therein, amino acids 26-109 (numbered based on the precursor protein of SEQ ID NO: 19) constitute the F2 chain (set forth in SEQ ID NO: 40), amino acids 110-136 constitute the p27 peptide (SEQ ID NO: 42) and amino acids 137-574 constitute the F1 chain (set forth in SEQ ID NO: 39). The F1 polypeptide included in the modified F protein may be a variant of SEQ ID NO: 39 or a polypeptide which corresponds to the F1 chain of SEQ ID NO: 39, or is a variant of a polypeptide corresponding to SEQ ID NO: 39. Similarly, the F2 polypeptide included in the modified F protein may be a variant of SEQ ID NO: 40 or a polypeptide which corresponds to the F2 chain of SEQ ID NO: 40, or is a variant of a polypeptide corresponding to SEQ ID NO: 40.

By a polypeptide which “corresponds to” the F1 or F2 chain of SEQ ID NO: 39 or 40 is meant a polypeptide from an RSV F protein which aligns to SEQ ID NO: 39 or SEQ ID NO: 40 when that F protein is aligned with the F protein of SEQ ID NO: 19. A variant of SEQ ID NO:39 may have at least 70, 75, 80, 85, 90 or 95% sequence identity to SEQ ID NO: 39. Similarly, a variant of SEQ ID NO: 40 may have at least 70, 75, 80, 85, 90 or 95% sequence identity to SEQ ID NO: 40.

As noted above, the modified F protein used herein contains a number of mutations relative to a wild-type sequence. When the amino acid position numbering of these mutations is based on SEQ ID NO: 19, the mutated amino acid positions correspond to the positions of the same numbers in SEQ ID NO: 19. Corresponding amino acid positions can be identified by sequence alignment, i.e. the position in an F protein sequence of interest corresponding to position 155 of SEQ ID NO: 19 is the position which corresponds to (or aligns to) position 155 of SEQ ID NO: 19 when the F protein sequence of interest is aligned to SEQ ID NO: 19.

As set out above, at least the F1 chain of the modified F protein comprises mutations relative to a wild type F protein sequence. These mutations may act to stabilise the protein in its pre-F conformation. They may also or alternatively enhance the immune response against the protein, in particular enhance the pre-F-specific immune response against the protein. Techniques by which these characteristics of modified F proteins are known in the art and include e.g. the level of recognition by pre-F-specific antibodies, as described in Joyce et al (supra), incorporated herein by reference.

As is apparent from the paragraphs above, the F2 protein is truncated at its C-terminus relative to SEQ ID NO: 40, and the F1 protein is truncated at its N-terminus relative to SEQ ID NO: 39. Specifically, the F2 protein has a 6 amino acid C-terminal truncation, corresponding to the deletion of amino acids 104-109 of SEQ ID NO: 19, and the F1 protein has an 8 amino acid N-terminal truncation, corresponding to the deletion of amino acids 137-144 of SEQ ID NO: 19. The modified RSV F protein does not contain the p27 peptide, yielding in combination the above-mentioned deletion of positions corresponding to 104-144 of SEQ ID NO: 19. In light of this deletion, the amino acid at the position corresponding to 103 of SEQ ID NO: 19 is fused to the amino acid at the position corresponding to 145 of SEQ ID NO: 19. A glycine-serine (GS) linker is used to join the two positions. Modified RSV F proteins containing this deletion and linker are described in Joyce et al. (supra), where they are referred to as sc9-10 variants of DS-Cav1.

Of the above-mentioned mutations present in the modified F protein set out above, the S190F and V207L mutations are cavity-filling substitutions, while the S155C and S290C mutations provide an additional disulphide bond. These four mutations are the DS-Cav1 mutations and stabilise the protein in its pre-fusion conformation. The pre-fusion F protein ectodomain comprises a cavity which collapses during transition to the post-fusion conformation. Filling of the cavity with bulky amino acid side chains acts against transition to the post-fusion conformation. An additional disulphide bond is also provided by the cysteine residues introduced by the N183GC and N428C substitutions, further stabilising the pre-fusion conformation. Meanwhile, residue L373 natively interacts with L141 and L142, but their deletion leaves L373 exposed. The L373R mutation introduces a hydrophilic side chain at this location which also improves stability.

Where the modified F protein is based on an F protein other than that of SEQ ID NO: 19, but the amino acid numbering is based on SEQ ID NO: 19, the modified F protein has the specified amino acid residues at the specified positions. For example, a modified F protein with an S155C mutation, based on the numbering of SEQ ID NO: 19, has a cysteine residue at the amino acid position corresponding to position 155 of SEQ ID NO: 19. It is unimportant whether this cysteine residue has been introduced as a result of a Ser=>Cys mutation, a different mutation, or is natively located at that position in the wild type F protein.

The modified RSV F protein may also comprise one or more additional substitutions in the F1 and/or F2 chains relative to SEQ ID NO: 19, in addition to the S155C, N183GC, S190F, V207L, S290C, L373R and N428C mutations specified above. In particular, the modified RSV F protein may further comprise one or more substitutions at positions S46, E92 and S215. For instance, the modified RSV F protein may comprise one or more of the following substitutions: S46G, E92D and S215P. For example, the modified RSV F protein may comprise 1, 2 or 3 of these substitutions.

In particular instances, the modified RSV F protein comprises only the S155C, N183GC, S190F, V207L, S290C, L373R and N428C substitutions. In other instances, the modified RSV F protein comprises the S46G, E92D, S155C, N183GC, S190F, V207L, S215P, S290C, L373R and N428C substitutions.

Other mutations may also be present in the modified F protein. For instance, in some instances, the modified RSV F protein additionally comprises A149C and Y458C substitutions, yielding an additional disulphide bond. In some instances, the modified RSV F protein additionally comprises T369C and T455C substitutions, yielding an additional disulphide bond. In some instances, the modified RSV F protein additionally comprises cysteine substitutions at one of positions 98-100 and one of positions 361-362, e.g. Q98C and Q361C substitutions, S99C and Q361C substitutions, or T100C and S362C substitutions (or any other combination thereof), yielding an additional disulphide bond.

Other mutations which may be introduced, in any combination, include N67I, L95M, I217P, I221M, R429K and K465Q. In some instances, the modified F protein comprises the L95M, I221M, R429K and, optionally, I217P mutations. In some instances, the modified F protein comprises the N67I and K465Q mutations. Such mutations may increase the stability and/or expression of the modified F protein.

The pre-fusion conformation comprises an antigenic site which, in its native form, is located at its membrane distal apex, termed Ø. Antigenic site Ø includes RSV F residues 62-69 and 196-209, but is lost from the post-fusion conformation. Several pre-fusion-specific antibodies bind this site. Stabilisation of the F protein in its pre-fusion domain preserves or stabilises antigenic site Ø, and thus pre-fusion stabilised F proteins can be identified as those that are recognised by pre-fusion specific antibodies such as AM22, D25 and 5C4 (McLellan et al., supra). Typically, a pre-fusion-stabilised RSV F protein specifically binds to a pre-fusion specific antibody such as these with a dissociation constant of less than about 10⁻⁶ M, such as less than about 10⁻⁷ M, 10⁻⁸ M or 10⁻⁹ M.

The pre-fusion-stabilised RSV F protein may retain specific binding for a pre-fusion-specific antibody (such as those mentioned above) following incubation at 70° C. for 30 minutes or one hour in phosphate buffered saline (PBS). For example, a pre-fusion-stabilised RSV F protein may retain at least 50, 60 or 70% binding to a pre-fusion-specific antibody after such an incubation.

Alternatively or additionally, the pre-fusion-stabilised RSV F protein may retain specific binding for a pre-fusion-specific antibody (such as those mentioned above) following incubation at 4° C. for 1, 2, 3, 4, 5 or 6 months in PBS. For example, a pre-fusion-stabilised RSV F protein may retain at least 50, 60, 70, 80 or 90% binding to a pre-fusion-specific antibody after such an incubation.

Thus in the pre-fusion-stabilised RSV F protein, one or more antigenic epitopes are accessible on the protein surface. In particular one or more neutralising epitopes are accessible on the protein surface. The accessible epitopes may be pre-fusion specific epitopes, i.e. epitopes which are accessible in the pre-fusion conformation of the F protein, but not in the post-fusion conformation. For example, the antigenic site Ø may be accessible. By “accessible” herein is meant accessible to antibodies, such that an epitope can be bound by an antibody.

The F2 chain of the modified RSV F protein may have an N-terminal position corresponding to one of RSV F positions 20-30, e.g. 25-30, such as position 25, 26, 27, 28, 29 or 30. In particular instances, the F2 chain has an N-terminal position corresponding to position 26 of SEQ ID NO: 19 (i.e. the N-terminal residue of the F2 chain from the native SEQ ID NO: 19 F protein). The F1 chain of the modified RSV F protein may have a C-terminal position corresponding to one of RSV F positions 510-525, e.g. one of positions 510-520 or 510-515 of RSV F of SEQ ID NO: 19, such as position 510, 511, 512, 513, 514 or 515. In particular instances, the F1 chain has a C-terminal position corresponding to position 513 of SEQ ID NO: 19.

In some instances, the F1 chain comprises the amino acid sequence set forth in SEQ ID NO: 45, or a variant thereof having at least 80, 85, 90 or 95% identity thereto, subject to the proviso that a variant thereof comprises the S155C, N183GC, S190F, V207L, S290C, L373R and N428C substitutions (numbered according to SEQ ID NO: 19). SEQ ID NO: 45 corresponds to amino acids 145-513 of SEQ ID NO: 19, with the S155C, N183GC, S190F, V207L, S290C, L373R and N428C substitutions. In some instances, the variant of SEQ ID NO: 45 is SEQ ID NO: 46, which corresponds to SEQ ID NO: 45 and additionally comprises the S215P substitution. In some instances, the F1 chain comprises the amino acid sequence set forth in SEQ ID NO: 46, or a variant thereof having at least 80, 85, 90 or 95% identity thereto, subject to the proviso that a variant thereof comprises the S155C, N183GC, S190F, V207L, S215P, S290C, L373R and N428C substitutions (numbered according to SEQ ID NO: 19).

In some instances, the F2 chain comprises the amino acid sequence set forth in SEQ ID NO: 47, or a variant thereof having at least 80, 85, 90 or 95% identity thereto. SEQ ID NO: 47 corresponds to amino acids 26-103 of SEQ ID NO: 19. In some instances, the variant of SEQ ID NO: 47 is SEQ ID NO: 48, which corresponds to SEQ ID NO: 47 and additionally comprises the S46G and E92D substitutions. In some instances, the F2 chain comprises the amino acid sequence set forth in SEQ ID NO: 48, or a variant thereof having at least 80, 85, 90 or 95% identity thereto, subject to the proviso that a variant thereof comprises the S46G and E92D substitutions.

The modified F protein encoded by the mRNA molecule provided herein may comprise an F1 chain comprising or consisting of the amino acid sequence set forth in SEQ ID NO: 45 or 46, or a variant thereof, and an F2 chain comprising or consisting of the amino acid sequence set forth in SEQ ID NO: 47 or 48, or a variant thereof, in particular an F1 chain of SEQ ID NO: 45 or 46 and an F2 chain of SEQ ID NO: 47 or 49.

For instance, the modified F protein may comprise an F1 chain comprising or consisting of the amino acid sequence set forth in SEQ ID NO: 45, or a variant thereof, and an F2 chain comprising or consisting of the amino acid sequence set forth in SEQ ID NO: 47, or a variant thereof, in particular an F1 chain of SEQ ID NO: 45 and an F2 chain of SEQ ID NO: 47; or, the modified F protein may comprise an F1 chain comprising or consisting of the amino acid sequence set forth in SEQ ID NO: 46, or a variant thereof, and an F2 chain comprising or consisting of the amino acid sequence set forth in SEQ ID NO: 48, or a variant thereof, in particular an F1 chain of SEQ ID NO: 46 and an F2 chain of SEQ ID NO: 48.

Alternatively, the modified F protein may comprise an F1 chain comprising or consisting of the amino acid sequence set forth in SEQ ID NO: 45, or a variant thereof, and an F2 chain comprising or consisting of the amino acid sequence set forth in SEQ ID NO: 48, or a variant thereof, in particular an F1 chain of SEQ ID NO: 45 and an F2 chain of SEQ ID NO: 48; or, the modified F protein may comprise an F1 chain comprising or consisting of the amino acid sequence set forth in SEQ ID NO: 46, or a variant thereof, and an F2 chain comprising or consisting of the amino acid sequence set forth in SEQ ID NO: 47, or a variant thereof, in particular an F1 chain of SEQ ID NO: 46 and an F2 chain of SEQ ID NO: 47.

The modified F protein may further comprise an N-terminal signal sequence to direct secretion from cells in which it is expressed. Any signal sequence may be used, though conveniently the signal sequence of SEQ ID NO: 41 may be used, or a variant thereof having at least 80, 85, 90 or 95% identity to SEQ ID NO: 41.

A modified RSV F protein consisting of the signal peptide of SEQ ID NO: 41, the F1 chain of SEQ ID NO: 45 and the F2 chain of SEQ ID NO: 47 has the amino acid sequence set out in SEQ ID NO: 2.

A modified RSV F protein consisting of the signal peptide of SEQ ID NO: 41, the F1 chain of SEQ ID NO: 46 and the F2 chain of SEQ ID NO: 50 has the amino acid sequence set out in SEQ ID NO: 1. This modified RSV F protein is described in Joyce et al. (supra) where it is referred to as sc9-10 DS-Cav1 N183GC N428C S46G S215P E92D.

The modified RSV F protein of SEQ ID NO: 1 may be referred to herein as DS2-2, and the modified RSV F protein of SEQ ID NO: 2 as DS2-2(A).

In particular instances, the modified RSV F protein comprises:
(i) the amino acid sequence set forth in SEQ ID NO: 1; or
(ii) a variant of SEQ ID NO: 1 having at least 80, 85, 90 or 95% identity thereto, but comprising:
a) a glycine residue at the position corresponding to position 46 of SEQ ID NO: 1;
b) an aspartic acid residue at the position corresponding to position 92 of SEQ ID NO: 1;
c) a cysteine residue at the position corresponding to position 116 of SEQ ID NO: 1;
d) a glycine residue at the position corresponding to position 144 of SEQ ID NO: 1;
e) a cysteine residue at the position corresponding to position 145 of SEQ ID NO: 1;
f) a phenylalanine residue at the position corresponding to position 152 of SEQ ID NO: 1;
g) a leucine residue at the position corresponding to position 169 of SEQ ID NO: 1;
h) a proline residue at the position corresponding to position 177 of SEQ ID NO: 1;
i) a cysteine residue at the position corresponding to position 252 of SEQ ID NO: 1;
j) an arginine residue at the position corresponding to position 335 of SEQ ID NO: 1; and;
k) a cysteine residue at the position corresponding to position 390 of SEQ ID NO: 1.

In other particular instances, the modified RSV F protein comprises:
(i) the amino acid sequence set forth in SEQ ID NO: 2; or
(ii) a variant of SEQ ID NO: 2 having at least 80, 85, 90 or 95% identity thereto, but comprising:
a) a serine residue at the position corresponding to position 46 of SEQ ID NO: 1;
b) a glutamic acid residue at the position corresponding to position 92 of SEQ ID NO: 1;
c) a cysteine residue at the position corresponding to position 116 of SEQ ID NO: 1;
d) a glycine residue at the position corresponding to position 144 of SEQ ID NO: 1;
e) a cysteine residue at the position corresponding to position 145 of SEQ ID NO: 1;
f) a phenylalanine residue at the position corresponding to position 152 of SEQ ID NO: 1;
g) a leucine residue at the position corresponding to position 169 of SEQ ID NO: 1;
h) a serine residue at the position corresponding to position 177 of SEQ ID NO: 1;
i) a cysteine residue at the position corresponding to position 252 of SEQ ID NO: 1;
j) an arginine residue at the position corresponding to position 335 of SEQ ID NO: 1; and;
k) a cysteine residue at the position corresponding to position 390 of SEQ ID NO: 1.

It should be noted that positions 46, 92, 116, 144, 145, 152, 169, 177, 252, 335 and 390 of SEQ ID NOs: 1 and 2 correspond to positions 46, 92, 155, 183, 190, 207, 215, 290, 373 and 428, respectively, of the reference sequence of SEQ ID NO: 19 (both positions 144 and 145 of SEQ ID NOs: 1 and 2 correspond to position 183 of SEQ ID NO: 19).

An amino acid position in a variant of SEQ ID NO: 1 or 2 that corresponds to a particular position in SEQ ID NO: 1 or 2 is the position in the variant that aligns to the relevant position of SEQ ID NO: 1 or 2, if the sequence of the variant is aligned with SEQ ID NO: 1 or 2. Thus for example, if a variant of SEQ ID NO: 1 is aligned with SEQ ID NO: 1, the position in the variant that corresponds to position 46 of SEQ ID NO: 1 is the position in the variant that aligns to position 46 of SEQ ID NO: 1. The position corresponding to position 46 of SEQ ID NO: 1 may also be at position 46 in the variant, but may be at a different position if the variant contains an insertion or deletion mutation relative to SEQ ID NO: 1 prior to that position.

In particular instances, the immunogen comprises or consists of the amino acid sequence set forth in SEQ ID NO: 1. The examples below show that use of SEQ ID NO: 1 as an antigen in an animal model of RSV vaccination yields particularly high levels of neutralising antibodies.

In other particular instances, the immunogen comprises or consists of the amino acid sequence set forth in SEQ ID NO: 2.

Where the immunogen is a variant of SEQ ID NO: 1 or SEQ ID NO: 2 at least one of the accessible epitope sequences (particularly neutralising epitope sequences) is unaltered relative to SEQ ID NO: 1 or SEQ ID NO: 2 or is sufficiently unaltered that upon administration to subjects, the variant induces at least similar antibody (or neutralising antibody) titres to the wild type epitope sequence, e.g. antibody (or neutralising antibody) titres which are at least 70, 75, 80, 85, 90, 95, 100, 105, 110, 115 or 120% or more as high as the titres obtained with the specific F protein antigens of SEQ ID NOs: 1 and 2. Variants of SEQ ID NO: 1 or SEQ ID NO: 2 induce production of antibodies which recognise wild type RSV F protein, i.e. the amino acid mutations relative to SEQ ID NO: 1 or 2 do not reduce the affinity of the antibodies produced for the native F protein.

In particular instances, the fusion protein which forms the nanoparticle subunit comprises or consists of the amino acid sequence set forth in SEQ ID NO: 6, or a variant thereof having at least 80, 85, 90 or 95% identity to SEQ ID NO: 6. SEQ ID NO: 6 is a fusion protein comprising, from N-terminus to C-terminus, the modified RSV F protein of SEQ ID NO: 1, the linker of SEQ ID NO: 4 and the lumazine synthase scaffold of SEQ ID NO: 3. In particular instances the fusion protein comprises or consists of SEQ ID NO: 6.

In particular instances, the fusion protein comprises or consists of the amino acid sequence set forth in SEQ ID NO: 7, or a variant thereof having at least 80, 85, 90 or 95% identity to SEQ ID NO: 7. SEQ ID NO: 7 is a fusion protein comprising, from N-terminus to C-terminus, the modified RSV F protein of SEQ ID NO: 2, the linker of SEQ ID NO: 4 and the lumazine synthase scaffold of SEQ ID NO: 3. In particular instances the fusion protein comprises or consists of SEQ ID NO: 7.

The nucleotide sequences used to encode the immunogen and the scaffold may be the native sequences (or in the case of the modified F protein, modified relative to the native sequence only where necessary due to the amino acid sequence modifications relative to the native F protein). Alternatively, the nucleotide sequences may be codon-optimised for human expression relative to the native sequences (particularly in the case of the sequence encoding the lumazine synthase, which is derived from a non-human species).

RNA nucleotide sequences encoding the immunogens of SEQ ID NOs: 1 and 2, respectively, are set forth in SEQ ID NOs: 8 and 9. An RNA nucleotide sequence of the lumazine synthase of SEQ ID NO: 3 is set forth in SEQ ID NO: 10 and an RNA sequence of the linker of SEQ ID NO: 4 is set forth in SEQ ID NO: 11. In the mRNA molecule provided herein, the immunogen may be encoded by the nucleotide sequence set forth in SEQ ID NO: 8 or 9, or a nucleotide sequence which is degenerate with SEQ ID NO: 8 or 9, or a nucleotide sequence which encodes a modified F protein as set out above and has at least 90 or 95% identity to SEQ ID NO: 8 or 9; the scaffold may be encoded by the nucleotide sequence set forth in SEQ ID NO: 10, or a nucleotide sequence which is degenerate with SEQ ID NO: 10, or a nucleotide sequence which encodes a lumazine synthase as set out above and has at least 90 or 95% identity to SEQ ID NO: 10; the linker may be encoded by the nucleotide sequence set forth in SEQ ID NO: 11, or a nucleotide sequence which is degenerate with SEQ ID NO: 11, or a nucleotide sequence which encodes a linker of suitable length for use herein and has at least 90 or 95% identity to SEQ ID NO: 11.

The nucleotide sequence in the mRNA molecule which encodes the immunogen may be referred to as the immunogen coding sequence. Equivalently, the nucleotide sequence which encodes the linker may be referred to as the linker coding sequence, and the nucleotide sequence which encodes the scaffold may be referred to as the scaffold coding sequence. As set out above, the mRNA molecule may comprise: an immunogen coding sequence comprising or consisting of the nucleotide sequence set forth in SEQ ID NO: 8 or SEQ ID NO: 9, a nucleotide sequence which is degenerate with SEQ ID NO: 8 or SEQ ID NO: 9 or a nucleotide sequence which encodes a modified F protein as set out above and has at least 90 or 95% identity to SEQ ID NO: 8 or 9; a scaffold coding sequence comprising or consisting of the nucleotide sequence set forth in SEQ ID NO: 10, a nucleotide sequence which is degenerate with SEQ ID NO: 10 or a nucleotide sequence which encodes a lumazine synthase as set out above and has at least 90 or 95% identity to SEQ ID NO: 10; and a linker coding sequence comprising or consisting of the nucleotide sequence set forth in SEQ ID NO: 11, a nucleotide sequence which is degenerate with SEQ ID NO: 11, or a nucleotide sequence which encodes a linker of suitable length for use herein and has at least 90 or 95% identity to SEQ ID NO: 11.

A nucleotide sequence encoding the fusion protein of SEQ ID NO: 6 is provided as SEQ ID NO: 13, and a nucleotide sequence encoding the fusion protein of SEQ ID NO: 7 is provided as SEQ ID NO: 14. In the mRNA molecule provided herein, the fusion protein may be encoded by the nucleotide sequence of SEQ ID NO: 13 or SEQ ID NO: 14, a nucleotide sequence degenerate with SEQ ID NO: 13 or SEQ ID NO: 14 or a nucleotide sequence which encodes a fusion protein as set out herein and has at least 90 or 95% identity to SEQ ID NO: 13 or SEQ ID NO: 14. In particular instances the fusion protein is encoded by SEQ ID NO: 13 or SEQ ID NO: 14. The nucleotide sequence which encodes the fusion protein in the mRNA molecule may be referred to as the fusion protein coding sequence or nanoparticle subunit coding sequence. Thus the mRNA molecule provided herein may comprise a nanoparticle subunit coding sequence comprising or consisting of the nucleotide sequence of SEQ ID NO: 13 or SEQ ID NO: 14, a nucleotide sequence degenerate with SEQ ID NO: 13 or SEQ ID NO: 14 or a nucleotide sequence which encodes a nanoparticle subunit as set out herein and has at least 90 or 95% identity to SEQ ID NO: 13 or SEQ ID NO: 14.

In particular instances, the mRNA molecule provided herein comprises or consists of the nucleotide sequence set out in SEQ ID NO: 17 or SEQ ID NO: 18, or a nucleotide sequence having at least 90 or 95% identity thereto and which encodes a fusion protein as set out above. SEQ ID NO: 17 contains, 5′ to 3′, the 5′ UTR of SEQ ID NO: 15, the fusion protein gene of SEQ ID NO: 13 and the 3′ UTR of SEQ ID NO: 16. SEQ ID NO: 18 contains, 5′ to 3′, the 5′ UTR of SEQ ID NO: 15, the fusion protein gene of SEQ ID NO: 14 and the 3′ UTR of SEQ ID NO: 16.

In a particular instance the mRNA molecule provided herein comprises or consists of the nucleotide sequence set forth in SEQ ID NO: 17.

In another particular instance the mRNA molecule provided herein comprises or consists of the nucleotide sequence set forth in SEQ ID NO: 18.

### Vaccines

Further provided herein is a vaccine comprising the mRNA molecule described above, specifically an RSV vaccine (i.e. an RSV mRNA vaccine).

In the vaccine, the mRNA molecule may be formulated in any suitable delivery vehicle, in particular a lipid-based delivery system such as a lipid nanoparticle, a liposome or a lipoplex. Lipid nanoparticles may be particularly suitable.

### Lipid Nanoparticles

In the vaccines provided herein, the mRNA molecule is generally formulated in a lipid nanoparticle. A lipid nanoparticle is an essentially spherical particle with a surface comprising a lipid layer surrounding a core. The lipid layer may be a monolayer or a bilayer. The core may be lipophilic, such as a solid lipid matrix, and may be stabilised by surfactants.

Lipid nanoparticle (LNP) characteristics and behaviour in vivo can be modified by addition of a hydrophilic polymer coating, e.g. polyethylene glycol (PEG), to the surface. Furthermore, LNPs can be used for specific targeting by attaching ligands (e.g., antibodies, peptides, or carbohydrates) to their surface or to the terminal end of the attached PEG chains (Front Pharmacol. 2015 Dec. 1; 6:286).

The mRNA molecule formulated in a lipid nanoparticle is generally encompassed by the lipid nanoparticle, i.e. the mRNA molecule is located in the core of the nanoparticle, entirely surrounded by lipid and is inaccessible to external milieu. In the core, cationic and/or ionisable lipids can organise into inverted micelles around, the encapsulated, negatively charged mRNA molecules.

Lipid nanoparticles (LNPs) as used herein may comprise a cationic lipid and an aggregation reducing agent (such as a PEGylated lipid, also referred to herein as a polyethylene glycol (PEG)-modified lipid or a PEG-lipid), and optionally a non-cationic lipid (such as a neutral lipid) and/or a sterol. Generally, the LNP comprises a cationic lipid, a non-cationic lipid, a PEG-lipid and a sterol.

LNPs may include any cationic lipid suitable for forming a lipid nanoparticle. The cationic lipid may carry a net positive charge at physiological pH. The cationic lipid may comprise a fatty acid chain of any suitable length, e.g. C10, C12 or C14 to C22 or C24. The fatty acid chain may be saturated, monounsaturated or polyunsaturated (used herein to mean comprising two or more carbon-carbon double bonds).

The cationic lipid may be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

Suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some instances, amino or cationic lipids have at least one protonatable or deprotonatable group (which may also be referred to as ionisable lipids), such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention.

In some instances, the protonatable lipids have a pKa of the protonatable group in the range of about 4 to about 11, e.g. a pKa of about 5 to about 7.

LNPs can include two or more cationic lipids. The cationic lipids can be selected to contribute different advantageous properties. For example, cationic lipids that differ in properties such as amine pKa, chemical stability, half-life in circulation, half-life in tissue, net accumulation in tissue or toxicity can be used in the LNP. In particular, the cationic lipids can be chosen so that the properties of the mixed-LNP are more desirable than the properties of a single-LNP of individual lipids. In some instances, the ratio of cationic lipid to nucleic acid in the LNP is from about 3 to about 15, such as from about 5 to about 13 or from about 7 to about 11.

The non-cationic lipid can be a neutral lipid, an anionic lipid, or an amphipathic lipid. Neutral lipids, when present, can be any of a number of lipid species which exist either in an uncharged or neutral zwitterionic form at physiological pH. The selection of neutral lipids for use in the particles described herein is generally guided by consideration of, e.g., LNP size and stability of the LNP in the bloodstream.

Neutral lipids may comprise a fatty acid chain of any suitable length, e.g. C10, C12 or C14 to C22 or C24. The fatty acid chain may be saturated, monounsaturated or polyunsaturated. In some instances, the neutral lipids contain saturated fatty acids with carbon chain lengths in the range of C10 to C20. In other instances, neutral lipids with monounsaturated or diunsaturated fatty acids with carbon chain lengths in the range of C10 to C20 are used. Additionally, neutral lipids having mixtures of saturated and unsaturated fatty acid chains can be used.

Amphipathic lipids are those in which the hydrophobic portion of the lipid material orients into a hydrophobic phase, while the hydrophilic portion orients towards the aqueous phase. Such compounds include, but are not limited to, phospholipids, aminolipids, sphingolipids, glycosphingolipids, diacylglycerols and beta-acyloxyacids.

The sterol is generally cholesterol.

The aggregation reducing agent can be a lipid capable of reducing aggregation. Examples of such lipids include, but are not limited to, polyethylene glycol (PEG)-modified lipids, monosialoganglioside Gml, and polyamide oligomers (PAO). Other compounds with uncharged, hydrophilic, steric-barrier moieties, which prevent aggregation during formulation, like PEG, Gml or ATTA, can also be coupled to lipids.

The composition of LNPs may be influenced by, inter alia, the selection of the cationic lipid component, the degree of cationic lipid saturation, the nature of the PEGylation, the ratio of all components and biophysical parameters such as its size.

In some instances, the LNPs comprise a cationic lipid, a non-cationic lipid, a PEG-lipid and cholesterol.

In some instances, LNPs may comprise about 35 to 45%, 40 to 50%, 40 to 60%, 50 to 60% or 55 to 65% cationic lipid on a molar basis (i.e. mol %). In some instances, the cationic lipid is present in a ratio of from about 50 mol % to about 60, 65, 70, 75, 80, 85, 90 or 95 mol % of the total lipid present in the LNP, e.g. from about 60 mol % to about 70, 75, 80, 85, 90 or 95 mol % of the total lipid present in the LNP, or from about 70 mol % to about 80, 85, 90 or 95 mol % of the total lipid present in the LNP. The cationic lipid may be present in a ratio of about 50, 55, 60, 65, 70, 75, 80, 85, 90 or 95 mol % of the total lipid present in the LNP.

In some instances, LNPs may comprise about 1 to 25%, 1 to 20%, 1 to 15%, 1 to 10%, 3 to 25%, 3 to 20%, 3 to 15%. 3 to 10%, 5 to 25%, 5 to 20%, 5 to 15% or 5 to 10% non-cationic lipid on a molar basis (i.e. mol %). In some instances, the non-cationic lipid is present in a ratio of from about 5 mol % to about 90 mol %, 50 mol %, 40 mol %, 30 mol %, 20 mol %, 15 mol % or about 10 mol % of the total lipid present in the LNP, e.g. from about 10 mol % to about 90 mol %, 50 mol %, 40 mol %, 30 mol % or 20 mol % of the total lipid present in the LNP. The non-cationic lipid may be present in a ratio of about 5, 10, 15, 20, 25, 30, 35 or 40 mol % of the total lipid present in the LNP.

In some instances, the molar ratio or weight ratio of lipid to mRNA in the nanoparticle ranges from about 5:1 to about 20:1, from about 10:1 to about 25:1, from about 15:1 to about 30:1, or at least 30:1.

In some instances, the LNPs may comprise about 0.1 to 5% aggregation-reducing agent (e.g. PEG-lipid) on a molar basis (i.e. mol %), e.g. 0.1 to 4 mol %, 0.1 to 3 mol %, 0.1 to 2 mol %, 0.1 to 1 mol %, 0.5 to 5 mol %, 0.5 to 4 mol %, 0.5 to 3 mol %. 0.5 to 2 mol %, 0.5 to 1 mol %, 1 to 5 mol %, 1 to 4 mol %, 1 to 3 mol % or 1 to 2 mol % aggregation-reducing agent.

The average molecular weight of the PEG moiety in PEG-modified lipids can range from about 1000 to about 8000 Daltons (e.g., from about 1000 to about 4000 Daltons). In some instances, the average molecular weight of the PEG moiety is about 2000 Daltons.

In some instances, the LNPs may comprise about 10 to 70 mol % sterol (e.g. cholesterol), e.g. about 20 to 60 mol % or 30 to 50 mol % sterol.

In some instances, the LNPs may comprise about 40 to 60% cationic lipid, 5 to 15% non-cationic lipid, 1 to 2% PEG-lipid, and 30-50% cholesterol by weight.

In some instances, LNPs have a median diameter size of about 50 to 300 nm, such as about 50 to 250 nm, 50 to 200 nm, 50 to 150 nm, 100 to 300 nm, 100 to 250 nm, 100 to 200 nm or 150 to 300 nm.

Alternatively, nucleic acids may be delivered using smaller LNPs which may comprise a median diameter from about 1 to 100 nm, e.g. 1 to 10 nm, 1 to 20 nm, 1 to 30 nm, 1 to 40 nm, 1 to 50 nm, 1 to 60 nm, 1 to 70 nm, 1 to 80 nm, 1 to 90 nm, 5 to 100 nm, 5 to 10 nm, 5 to 20 nm, 5 to 30 nm, 5 to 40 nm, 5 to 50 nm, 5 to 60 nm, 5 to 70 nm, 5 to 80 nm, 5 to 90 nm, 10 to 50 nm, 20 to 50 nm, 30 to 50 nm, 40 to 50 nm, 20 to 60 nm, 30 to 60 nm, 40 to 60 nm, 20 to 70 nm, 30 to 70 nm, 40 to 70 nm, 50 to 70 nm, 60 to 70 nm, 20 to 80 nm, 30 to 80 nm, 40 to 80 nm, 50 to 80 nm, 60 to 80 nm, 20 to 90 nm, 30 to 90 nm, 40 to 90 nm, 50 to 90 nm, 60 to 90 nm and/or 70 to 90 nm.

Alternatively, nucleic acids may be delivered using larger LNPs having a median diameter greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In other instances, LNPs have a single mode particle size distribution (i.e. they are not bi- or poly-modal).

LNPs may further comprise one or more lipids and/or other components in addition to those mentioned above. Other lipids may be included in the LNP compositions for a variety of purposes, such as to prevent lipid oxidation or to attach ligands onto the LNP surface. Any of a number of lipids may be present in LNPs, including amphipathic, neutral, cationic, and anionic lipids. Such lipids can be used alone or in combination. Additional components that may be present in a LNP include bilayer stabilizing components such as polyamide oligomers (see, e.g., U.S. Pat. No. 6,320,017, which is incorporated by reference in its entirety), peptides, proteins, and detergents.

### Liposomes

The mRNA molecule may be formulated in liposomes in the vaccine. Cationic lipid-based liposomes are able to complex with negatively charged nucleic acids (e.g. mRNA) via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the nucleic acid is then released from the endosome/carrier into the cytoplasm.

Liposomes typically consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposomes may have one or more lipid membranes. Liposomes can be single-layered, referred to as unilamellar, or multi-layered, referred to as multilamellar.

Liposome characteristics and behaviour in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes can be of different types and sizes, ranging from a small unicellular vesicle (SUV) which may be smaller than 100 nm in diameter, to a giant unicellular vesicle (GUV) which may be over a micrometre in diameter. Large unilamellar vesicles (LUVs) which are 100-1000 nm in diameter may also be used. Other suitable species of liposome include multilamellar vesicle (MLVs) which may be hundreds of nanometres or over a micron in diameter. MLVs contain a series of concentric bilayers separated by narrow aqueous compartments; and multivesicular liposomes (MVLs), in which multiple smaller, non-concentric vesicles are encapsulated within a single large vesicle, which may have a diameter of hundreds of nanometres or over a micron. Liposome design may include, but is not limited to, opsonins or ligands in order to improve the attachment of liposomes to a particular tissue or to activate events such as, but not limited to, endocytosis. Liposomes may contain a low or a high pH in order to improve the delivery of the pharmaceutical formulations.

### Lipoplexes

Alternatively, the mRNA molecule may be formulated as lipoplexes, i.e. cationic lipid bilayers sandwiched between nucleic acid layers. Cationic lipids, such as described above, can form complexes or lipoplexes with negatively charged nucleic acids to form nanoparticles by electrostatic interaction.

Vaccine Compositions and their Administration

The vaccine provided herein may be provided in a liquid form, or in a lyophilised form for reconstitution in liquid. The vaccine components (e.g. mRNA molecule, optionally formulated in a lipid nanoparticle or suchlike as set out above) may be constituted (or reconstituted) in a suitable buffer, particularly a buffer suitable for injection, e.g. an aqueous solution such as Ringer's lactate solution, Ringer's solution or a phosphate buffer solution.

A buffer suitable for injection may be used as a carrier in the vaccine or simply for resuspending the vaccine or vaccine components. Such a buffer suitable for injection may contain salts selected from, for example, sodium chloride (NaCl), calcium chloride (CaCl) or potassium chloride (KCl), wherein further anions may be present additional to the chlorides. The injection buffer may be hypertonic, isotonic or hypotonic with reference to relevant bodily fluids such as blood, lymph or cytosolic liquids. Suitable concentrations may be selected so as not to lead to cell damage due to osmosis or other concentration effects.

Additionally, liquid vaccine compositions, may include one or more of the following: polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as dextrose. A parenteral preparation can be enclosed in ampoules, disposable syringes or multiple dose vials made of glass or plastic. Vaccine compositions are preferably sterile.

The choice of a pharmaceutically acceptable carrier or diluent for the vaccine is determined, in principle, by the manner, in which it is to be administered. The vaccine can be administered, systemically or locally, generally locally. Routes for systemic administration include, for example, transdermal, oral and parenteral routes, including subcutaneous, intravenous, intramuscular, intraarterial, intradermal and intraperitoneal injections and/or intranasal administration routes. Routes for local administration include, for example, topical, intradermal, transdermal, subcutaneous and intramuscular injections. In a particular instance, the vaccine is administered intramuscularly. Intramuscular administration is generally by injection, which may be via a needle or may be needle-free.

The vaccine composition may comprise an adjuvant. An adjuvant may be used in order to enhance the immunostimulatory properties of the vaccine provided herein. In this context, an adjuvant may be understood as any compound which initiates or increases an immune response by the innate immune system, i.e. a non-specific immune response, when administered to a subject. In other words, when administered, the vaccine provided herein initiates an adaptive immune response against the immunogen encoded by the mRNA molecule. Additionally, the vaccine may generate a (supportive) innate immune response due to addition of an adjuvant as defined herein to the vaccine.

In other instances, the vaccine does not comprise an adjuvant, or does not contain a dedicated adjuvant component, e.g. the vaccine may comprise an mRNA molecule carrier which also functions as an adjuvant. For example, lipid nanoparticles may have an immunostimulatory or adjuvant effect, and so a vaccine comprising an mRNA molecule formulated in an LNP may utilise the intrinsic adjuvant activity of the LNP, and not comprise an additional adjuvant component.

### Methods of Treatment

The mRNA molecule or vaccine provided herein may be used in therapy, in particular in a method of vaccination. Thus provided herein is an mRNA molecule or vaccine as described above for use as a therapeutic agent. In particular, the mRNA molecule or vaccine is provided for use as a prophylactic agent. A “therapeutic agent” as referred to herein is an agent used for treatment or prevention of an illness or condition; a “prophylactic agent” as referred to herein is an agent used in medicine for prophylaxis of an illness or condition. Specifically, the mRNA molecule or vaccine provided herein can be used for prophylaxis of disease caused by RSV infection.

Prophylaxis of disease caused by RSV infection is broadly defined herein as reducing the likelihood of an individual developing a disease caused by RSV infection or reducing the severity of (i.e. attenuating) that disease in the event that the individual develops a disease caused by RSV infection. A disease caused by RSV infection may have reduced severity if e.g. the individual has a reduced likelihood of death, hospitalisation, admission to intensive care or requiring mechanical ventilation; the individual has symptoms which are milder and have a reduced impact on the individual's daily life, e.g. reducing the likelihood of the individual being bedridden and increasing their ability to care for themselves or others, work, etc; or the individual recovers more quickly, e.g. requires a shorter stay in hospital, intensive care, period of mechanical ventilation, etc., compared to if they were not vaccinated, and/or compared to an equivalent unvaccinated individual. An equivalent unvaccinated individual is an individual of similar size and age, of the same sex and in a similar condition of general health.

A prophylactic agent, as used herein, may be administered to an individual who is at particular risk of developing a disease caused by RSV infection or, more commonly, suffering severely from a disease caused by RSV infection. For example, the individual may be at high risk of exposure to RSB, or may be at risk of developing severe illness if infected by RSV due to e.g. age and/or other health conditions. For instance, the prophylactic agent may be administered as part of a routine or regular schedule of immunisation for individuals of a particular age, for example to older adults over the age of e.g. 50, 60 or 70. The prophylactic agent may be administered repeatedly, e.g. annually, to boost protection against RSV. For example, an RSV vaccine may be administered to an at-risk individual, such as an elderly person, annually or every 2, 3, 4 or 5 years or more, as needed. Such a reduction in risk of developing a disease caused by RSV infection, or suffering severely from a disease caused by RSV infection, may be a reduction of e.g. at least 50, 60, 70, 80 or 90% compared to an unvaccinated individual.

In particular, provided herein is an mRNA molecule or vaccine as set out above for use in a method of preventing and/or attenuating an RSV infectious disease in a subject. An “RSV infectious disease” is a disease caused by an RSV infection. The mRNA molecule or vaccine for use according to this aspect may prevent RSV infection. Alternatively, the mRNA molecule or vaccine may not prevent RSV infection, but may prevent symptomatic disease in the case of infection, or when symptomatic disease occurs, may reduce the severity of the disease. That is to say, the mRNA molecule or vaccine may reduce the likelihood of the subject becoming infected with RSV, e.g. in a particular context when there is a risk of exposure to the virus, or in day-to-day life, compared to an unvaccinated subject. Similarly, the mRNA molecule or vaccine may reduce the risk of the subject developing symptomatic disease caused by RSV compared to an unvaccinated subject, or may reduce the risk of the subject developing a severe disease caused by RSV (e.g. a disease requiring hospitalisation) compared to an unvaccinated subject. Such reductions in risk may be e.g. of at least 50, 60, 70, 80 or 90% compared to an unvaccinated subject.

In particular, the RSV infectious disease which is prevented or attenuated using the mRNA molecule or vaccine provided herein may be a lower respiratory tract disease (LRTD) caused by RSV. As standard in the art, the lower respiratory tract may be defined as the respiratory tract from the vocal cords downwards, comprising the trachea, lungs, bronchi and bronchioles. Examples of LRTDs caused by RSV include bronchiolitis, bronchitis and pneumonia, particularly bronchiolitis and pneumonia.

The subject in which an RSV infectious disease, e.g. LRTD, is prevented or attenuated may be the subject to whom the mRNA molecule or vaccine is administered. The subject is generally a human patient, e.g. a person over the age of 1, 2, 5, 12, 16, 18 or 21. In particular, the subject may be an adult, generally an older person over the age of e.g. 50, 55, 60, 65 or 70. Alternatively or additionally, the subject may suffer from a health condition putting them at greater risk of serious disease from RSV infection, e.g. cancer, an immunodeficiency or a respiratory condition such as COPD or suchlike. In adults and older children (e.g. over the ag of 5, or over the age of 2), the LRTD prevented or attenuated may in a particular instance be pneumonia.

In other instances, the subject may be a small child, e.g. an infant, baby or toddler, e.g. a child in the age range from birth to 6 months, 12 months, 18 months or 24 months. In small children, the LRTD prevented or attenuated may in a particular instance be bronchiolitis.

Alternatively, the subject in which an RSV infectious disease, e.g. LRTD, is prevented or attenuated may not be the same subject to whom the mRNA molecule or vaccine is administered. In particular, the subject protected by the vaccine (i.e. in which an RSV infectious disease is prevented or attenuated) may be infant child of a woman administered the mRNA molecule or vaccine prior to giving birth to the infant. In particular, the subject to whom the mRNA molecule or vaccine is administered may be a woman e.g. 20 to 38 weeks pregnant, e.g. 22-36 weeks, 24-36 weeks, 24-34 weeks, 24-32 weeks, 26-36 weeks, 26-34 weeks or 26-32 weeks pregnant. Vaccinating pregnant women in this way may lead to anti-RSV antibodies being passed to unborn children through the mother's placenta, providing passive protection against RSV infection and disease in the early months of a child's life. Such protection may be effective for at least e.g. 1, 2, 3, 4, 5 or 6 months after the birth of the child.

The above-discussed aspect may alternatively be seen as providing a method for preventing or attenuating an RSV infectious disease in a subject, particularly an LRTD caused by RSV. As detailed above, such a method may comprise administering the mRNA molecule or vaccine provided herein to the subject. Alternative, if the subject is an infant, the method may comprise administering the mRNA molecule or vaccine to the mother of the infant, while she is pregnant with the infant, as detailed above. Similarly, the above-discussed aspect may alternatively be seen as providing the use of an mRNA molecule or vaccine as described above in the manufacture of a medicament for use in preventing or attenuating a disease caused by an RSV infection in a subject, particularly an LRTD. The subject may be as described above.

More broadly, provided herein is a method of inducing an immune response in a subject, comprising administering to the subject an mRNA molecule or vaccine as described above. This aspect may alternatively be seen as providing an mRNA molecule or vaccine as described above for use in a method of inducing an immune response in a subject, or as providing the use of an mRNA molecule or vaccine as described above in the manufacture of a medicament for inducing an immune response in a subject. In this aspect, the immune response is an immune response against RSV, specifically against the RSV F protein, particularly the pre-fusion conformation of the F protein.

Such a specific immune response may entail a cellular (i.e. T cell) response and/or a humoral (i.e. antibody) response. Generally the immune response comprises at least an antibody response against the RSV F protein, i.e. the subject is induced to produce antibodies against the RSV F protein, particularly against pre-F RSV F. The antibodies produced by the subject may include neutralising antibodies against RSV F protein.

An antibody response induced by the mRNA molecule or vaccine may be identified and/or measured by ELISA (enzyme-linked immunosorbent assay). The size of an antibody response may be measured based on the antibody titre, e.g. by ELISA. An antibody titre is a measurement of the amount of antibodies within a subject, for example, antibodies that are specific to a particular antigen (e.g. the RSV F protein) or epitope of an antigen. Antibody titre is typically expressed as the inverse of the greatest dilution that provides a positive result.

In some instances, induction of an immune response results in an antibody titre in the subject which is increased by at least 1 order of magnitude relative to the titre prior to vaccination (i.e. relative to the titre in the same individual prior to vaccination). For example, the titre may be increased by at least 1.5, 2, 2.5 or 3 orders of magnitude relative to the titre prior to vaccination. In other instances, induction of an immune response results in an antibody titre in the subject which is increased at least 2-fold relative to the titre prior to vaccination. For example, the titre may be increased at least 3, 4, 5, 6, 7, 8 or 9-fold relative to prior to vaccination.

An antibody titre may measure the total amount of all antibodies against a particular antigen in a subject, or may measure just a particular type of antibody, e.g. neutralising antibody. In some instances, the increase in titre may be an increase in the titre of neutralising antibodies against RSV. The titre of neutralising antibodies against a virus, such as RSV, may be determined by a neutralisation assay. For instance, the titre of neutralising antibodies against RSV may be determined in an RSV neutralisation assay. A reliable neutralisation assay is the plaque reduction neutralisation test (PRNT), in which heat-inactivated serum of interest is applied with live virus to human cells. Viral infection of cells causes the formation of plaques in the cell culture dish, and the neutralising antibody titre can be calculated by the reduction in plaques. RSV neutralisation assays are described in detail in Raghunandan et al., Vaccine 39(33): 4591-4597, 2021, incorporated herein by reference.

The induced immune response may be long-lasting, e.g. the increased antibody titre or neutralising antibody titre may remain for at least 3, 4, 5, 6, 7, 8, 9, 10, 11, 12, 15 or 18 months or more after vaccination.

The induced immune response may include a memory response, i.e. a memory B cell response against RSV may be generated. Memory B cells may be identified by e.g. staining PBMCs with a fluorescent-labelled antigen (e.g. RSV F protein) and then identifying the antigen-specific memory B cells by flow cytometry based on binding of the fluorescent-labelled antigen and expression of memory B cell markers (e.g. CD27).

### Vectors and Cells

Also provided herein is a vector encoding the mRNA molecule provided herein. Such a vector is a DNA vector, and may be e.g. a cloning vector or an expression vector.

An “expression vector” as used herein is a DNA molecule used for expression of foreign genetic material in a cell or in an in vitro system. Any suitable vectors known in the art may be used. Suitable vectors include DNA plasmids, binary vectors, viral vectors and artificial chromosomes (e.g. yeast artificial chromosomes). An expression vector may be circular or linear. An expression vector comprises an expression cassette, from which the mRNA molecule provided herein may be expressed.

An “expression cassette” as used herein is a polynucleotide sequence that is capable of effecting transcription of an expression product. Typically, the expression cassette comprises a promoter operably linked to the sequence encoding the mRNA molecule. The term “operably linked” in this context means that the mRNA molecule sequence and promoter are covalently linked in such a way as to place the expression of the mRNA molecule under the influence or control of the promoter. Thus, a promoter is operably linked to the mRNA sequence if the promoter is capable of effecting transcription of the mRNA sequence in order to yield an mRNA molecule as described above.

Any suitable promoter known in the art may be used in the expression cassette providing it functions in the cell type being used. For example, where the cell is a mammalian cell, the promoter may be a cytomegalovirus (CMV) promoter. Where the expression cassette is to be expressed in vitro, the promoter may be a phage promoter, e.g. the SP6, T7 or T3 promoter. The promoter used in an expression cassette as provided herein may be a constitutive promoter or an inducible promoter.

An expression cassette may comprise additional elements useful for control of transcription, e.g. one or more enhancer sequences and one or more transcription termination (terminator) sequences.

The vector may additionally comprise other standard sequence elements such as a selectable marker, e.g. encoding an antibiotic resistance gene or suchlike.

Also provided herein is a cell comprising the vector provided herein. Such a cell may be e.g. a cloning host. Suitable cells include prokaryotic cells, e.g. bacterial cells, and eukaryotic cells, e.g. mammalian cells. Suitable bacterial cells include e.g. E. coli cells or B. subtilis cells.

mRNA Manufacture

The mRNA molecule provided herein may be manufactured in any suitable manner. The mRNA may be expressed in and purified from cells, e.g. mammalian cells, particularly human cells. Generally, the mRNA molecule is manufactured in vitro, in particular by in vitro transcription. The mRNA molecule may be transcribed from the expression vector described above.

Thus provided herein is a method of manufacturing the mRNA molecule provided herein, the method comprising expressing the mRNA molecule from the expression vector provided herein. Generally, the mRNA is expressed by in vitro transcription.

The expression vector used for in vitro transcription is generally linear, e.g. a linearised plasmid. Transcription may be performed using a phage RNA polymerase, e.g. the T7 RNA polymerase, SP6 RNA polymerase or T3 RNA polymerase. The expression vector contains a promoter recognised by the chosen polymerase. In vitro transcription can be performed using a commercially available kit in accordance with the manufacturer's instructions, e.g. a HiScribe® kit from New England Biolabs (NEB, USA) or a RiboMAX™ kit from Promega (USA).

### Protein Nanoparticles

Also provided herein is a protein nanoparticle comprising the nanoparticle subunit described above. The protein nanoparticle may be as described above in the context of the nanoparticle subunit encoded by the mRNA molecule.

The nanoparticle subunit may be expressed from an expression vector, as described above. Such an expression vector may be introduced into a host production cell, e.g. a mammalian cell, such as a human cell, and the protein expressed from it. The nanoparticle subunit may be expressed with a signal peptide so that it is exported from the cell and the multimer forms extracellularly, in which case the protein nanoparticle may be isolated from the culture supernatant. Otherwise, the multimer may form intracellularly, in which case the cells may be lysed to enable isolation of the protein nanoparticle. Cell lysis may be performed by any method known in the art, e.g. mechanical lysis using a French press, sonication or chemical lysis using a lysis agent.

Protein nanoparticle isolation/purification may be performed using standard methods in the art, e.g. the fusion protein may be expressed with an affinity tag (such as a His-tag) to enable affinity purification.

A protein nanoparticle as provided herein could be administered to a subject as described above as a protein vaccine.

Accordingly, in some instances, the disclosure provides a recombinant protein nanoparticle comprising 60 of the nanoparticle subunits described herein self-assembled into a nanoparticle. In some instances, when self-assembled, the nanoparticle subunits do not comprise a signal peptide.

### Sequence Identity and Variants

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equalling 12 and a gap extension penalty equalling 4. Use of GAP may be preferred but other algorithms may be used, e.g. BLAST (which uses the method of Altschul et al. (1990)), FASTA (which uses the method of Pearson and Lipman (1988)), or the Smith-Waterman algorithm (Smith and Waterman (1981)), the TBLASTN program, of Altschul et al. (1990) supra, or Emboss Needle (Madeira et al, Nucleic Acids Research 50(W1): W276-W279), generally employing default parameters.

Where the disclosure makes reference to a particular amino acid or nucleotide sequence having at least 90% sequence identity to a reference amino acid or nucleotide sequence, this includes the amino acid or nucleotide sequence having 90%, 91%, 92%, 93%, 94%, 95%, 96%, 97%, 98%, 99% and 100% sequence identity to the reference amino acid or nucleotide sequence (including as rounded to the nearest integer percentage).

Where an amino acid or nucleotide sequence has less than 100% identity to a reference sequence (e.g. about 90 or 95% identity to a reference sequence), that amino acid or nucleotide sequence comprises one or more sequence alterations relative to the reference sequence. The term “sequence alterations” as used herein is includes the substitution, deletion and/or insertion of an amino acid residue or nucleotide.

Thus, a protein containing one or more amino acid sequence alterations compared to a reference sequence contains one or more substitutions, one or more deletions and/or one or more insertions of an amino acid residue as compared to the reference sequence. In relation to peptide or polypeptide sequences, the term “amino acid mutation” is herein used interchangeably with “sequence alteration”, unless the context clearly identifies otherwise.

In some instances in which one or more amino acids are substituted with another amino acid, the substitutions may be conservative substitutions, for example according to the following table. In some instances, amino acids in the same block in the middle column are substituted, i.e. a non-polar amino acid is substituted for another non-polar amino acid for example. In some instances, amino acids in the same line in the rightmost column are substituted, i.e. G is substituted for A or P for example.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some instances, substitution(s) may be functionally conservative. That is, in some instances the substitution may not affect (or may not substantially affect) one or more functional properties of the protein containing the substitution as compared to the equivalent unsubstituted protein.

Similarly, a nucleic acid molecule (e.g. RNA molecule) containing one or more nucleotide sequence alterations compared to a reference sequence contains one or more substitutions, one or more deletions and/or one or more insertions of a nucleotide compared to the reference sequence. In relation to nucleic acid sequences, the term “nucleotide mutation” is herein used interchangeably with “sequence alteration”, unless the context clearly identifies otherwise.

Where a nucleic acid (e.g. RNA) sequence includes one or more sequence alterations, the alterations may similarly not affect (or not substantially affect) the functional properties of the nucleic acid. Where the nucleic acid sequence is a protein coding sequence, whether the functional properties of the sequence are affected by an alteration depends on whether the function or expression of the encoded protein is affected. Where the nucleic acid sequence is a non-coding nucleotide sequence, the effect of sequence alterations on the function of the non-coding sequence is assessed.

Nucleotide sequences may also be defined by degeneracy to a reference sequence. As a result of the degeneracy of the genetic code, there are many nucleotide sequences that may encode any given amino acid sequence. By degenerate nucleotide sequences is meant two (or more) nucleotide sequences which encode the same peptide or polypeptide (or amino acid sequence), specifically in the open reading frame of the reference nucleotide sequence which begins at position 1 (i.e. in which codon 1 of the encoding sequence corresponds to positions 1-3 of the reference nucleotide sequence). Thus for example, a nucleotide sequence degenerate with SEQ ID NO: 8 is a nucleotide sequence which is different to SEQ ID NO: 8 but which, due to the degeneracy of the genetic code, encodes the same protein sequence as SEQ ID NO: 8 (i.e. the modified RSV F protein of SEQ ID NO: 1).

The features disclosed in the foregoing description, or in the following claims, or in the accompanying drawings, expressed in their specific forms or in terms of a means for performing a disclosed function, or a method or process for obtaining the disclosed results, as appropriate, may be utilised separately, or in any combination of such features.

While exemplary instances are described above and below, many equivalent modifications and variations will be apparent to those skilled in the art. Accordingly, the exemplary instances set forth above and below are considered to be illustrative and not limiting. Various changes to the described instances may be made without departing from the spirit and scope of the present disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another instance includes from the one particular value and/or to the other particular value. Similarly, when values are expressed as approximations, by the use of the antecedent “about,” it will be understood that the particular value forms another instance. The term “about” in relation to a numerical value is optional and means for example +/−10%.

### EXAMPLES

The goal of the experiments outlined below was to develop an mRNA vaccine against respiratory syncytial virus (RSV) by targeting the Fusion (F) protein. Several preclinical and clinical trials have highlighted the importance of eliciting an antibody response towards the prefusion conformation of the F protein, specifically, against the prefusion specific sites 0 and 5. To accomplish this, various prefusion stabilized Fusion protein constructs, displayed on self-assembling protein nanoparticles (“nanoparticles”) were generated. Multiple nanoparticle scaffolds were evaluated, such as ferritin, a 24-mer derived from Helicobacter pylori; lumazine synthase (LuS), a 60-mer derived from Aquifex aeolicus; and beta-annulus (Bann), a 180-mer derived from Tomato bushy stunt virus. It was hypothesized that increasing the antigen density could occlude antigenic sites near the centre of the F trimer, while still exposing the apex of the trimer where site 0 resides.

### Example 1: Generation of Plasmid DNA Expressing RSV Prefusion F Displayed on Protein Nanoparticles and Expressed in HEK293 Cells

### Methods

### Prefusion F Cloning

Several prefusion F sequences (SEQ ID NOs in Table 1) were cloned into a pCAG DNA expression vector. The protein sequence of the prefusion stabilized F constructs (Table 2) were codon optimized and genetically linked at the C-terminus to a panel of self-assembling scaffolds. The sequences used to link the prefusion F with the scaffolds are listed in Table 3. The cloning was done using Gibson assembly.

TABLE 1

| Antigen | SEQ ID NO: |
|---|---|
| DS-Cav1 | 49 |
| DS2-2 | 1 |

TABLE 2

| Scaffold | SEQ ID NO: |
|---|---|
| Foldon | 50 |
| Ferritin | 51 |
| Ferritin with bullfrog leader | 52 |
| Lumazine synthase | 3 |
| Beta-annulus | 53 |

TABLE 3

| Antigen | SEQ ID NO: |
|---|---|
| DS2-2-LuS | 6 |
| DS-Cav1 with transmembrane domain | 54 |

### Transfection of Hek293 Cells

HEK293 cells were grown in T175 flasks in complete medium (DMEM supplemented with 10% FBS) at 37° C., 5% CO₂. Cells were harvested using trypsin for 5 minutes at 37° C. and plated at 300,000 cells per well in 24-well plates on day −1. On day 0, cells were transfected with pDNA (500 ng/well) encoding the RSV-F-linker scaffold antigens using Lipofectamine 2000 according to the manufacturer's protocol (Thermo Fisher Scientific, USA). On day 3, supernatant was harvested and evaluated by ELISA for production of RSV-F-nanoparticles.

### Sandwich ELISA

Supernatants from transfected cells were analysed by a sandwich ELISA. Briefly, 384-well ELISA plates were coated overnight at 4° C. with a panel of monoclonal antibodies (1G7, AM14, MPE8, Palivizumab, 101F and M43) at 3 μg/ml. Plates were washed and incubated with diluted cell supernatant for 1 hr at room-temperature. Plates were then washed and nanoparticles were detected with mAB 133-1H, a mouse antibody that recognizes both the prefusion and postfusion conformation of the F protein. Plates were washed and incubated with an anti-mouse-IgG-HRP antibody. Plates were washed and incubated with TMB substrate for 5 minutes and colour development was stopped with 2 N H₂SO₄ and plates were read with an Envision at 450 nm wavelength.

### Results

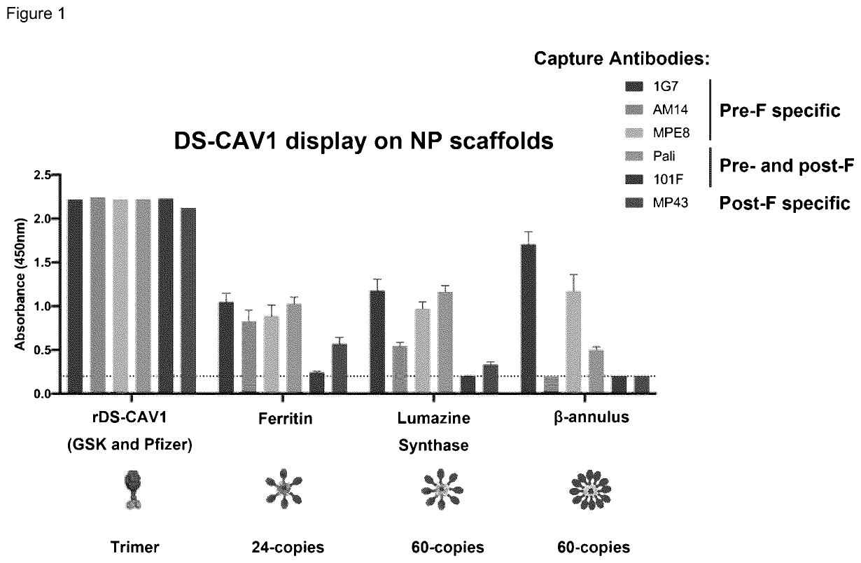

To characterise the antigenic sites maintained on the nanoparticles, HEK293 cells were transfected and supernatant was assessed by sandwich ELISA using a panel of capture antibodies that recognise various antigenic sites on RSV-F. Supernatant from cells transfected with DS-CAV1 displayed on ferritin, lumazine synthase (LuS) and beta-annulus (Bann) all produced nanoparticles that were captured with the prefusion specific mAB 1G7, indicating that site 0 was maintained and that the RSV-F protein displayed on the VLPs was in the prefusion conformation (FIG. 1). Additionally, DS-CAV1-ferritin and DS-CAV1-LuS all generated nanoparticles that could be captured using AM14 and MPE8, which recognize a quaternary epitope that spans multiple protomers. These results suggest that the nanoparticles are forming properly folded trimers in the prefusion conformation. Supernatant from cells transfected with DS-CAV1-Bann did not generate particles that could be bound by AM14 but could bind MPE8 (FIG. 1). The antigen density is greatest on Bann VLPs (180-mer).

DS-CAV1-ferritin and DS-CAV1-LuS particles could also be captured with palivizumab, but not mAB 101F (FIG. 1). These results suggest that the binding site for 101F could be occluded on these particles as well. Similarly, DS-CAV1-Bann nanoparticles could not bind 101F and had very low binding to palivizumab. The postfusion specific mAB M43 could bind DS-CAV1-ferritin and DS-CAV1-LuS particles, but not DS-CAV1-Bann nanoparticles. Overall, these results indicate that RSV-F can be displayed on nanoparticles and the antigen density on the surface can occlude certain antigenic sites, but all nanoparticles maintained accessibility to site 0.

### Example 2: DS2 Immunogens Elicit High-Titre Neutralising Antibodies in Naïve Mice

Mice were immunised with a panel of mRNAs encoding the prefusion stabilised constructs linked to the different scaffolds and the immunogenicity was evaluated by ELISA binding titres and neutralisation assays.

### Methods

mRNA Synthesis Using In Vitro Transcription (IVT)

Sequences encoding soluble DS-CAV1, membrane DS-Cav1 and DS2-nanoparticle subunits were cloned into an mRNA vector encoding the T7 promoter, 5′ and 3′ UTRs and a polyA tail. The template plasmid was linearised by digesting the pDNA with BSPQ1 (NEB, USA) for 4 h at 50° C. mRNAs were synthesized by in vitro transcription using NEB T7 HighScribe, as well as N1-methyl-pseudouridine and CleanCap-Ag (Trilink). IVT reactions were incubated for 5 hr at 37° C., followed by digestion with RNase free DNase. mRNA was then purified using LiCl precipitation for RNA purification. All mRNAs were formulated in an AZ lipid nanoparticle (LNP) in 20 mM Tris/Tris-HCL, pH 7.4, 8% w/v sucrose and then stored at −80° C.

### Mouse Immunizations

Mouse studies were approved by the Institutional Animal Care and Use Committee at AstraZeneca. Groups of 6 female BalB/c mice aged 5-7 weeks were immunised with the mRNA vaccines. LNP formulations were diluted in PBS according to the indicated dose. Mice were immunised subcutaneously with the diluted LNP in a 50 μl volume on day 0 and 28. Mice were bled on day 14 and 42 to evaluate the immune response.

### Binding ELISAs

ELISAs were performed to evaluate prefusion and postfusion antibody binding titres in immunised mice. 384-well plates were coated with 3 μg/ml of purified recombinant prefusion or postfusion F protein overnight at 4° C. Plates were then washed in PBS-T and blocked in PBS supplemented with 5% milk-powder and 3% BSA for 1 hr at room-temperature. Sera from mice was then diluted in blocking buffer and incubated with the coated plates for 1 hr at room-temperature. Plates were then washed and incubated with an HRP-conjugated anti-mouse IgG secondary antibody for 1 hr. Plates were washed and incubated with TMB substrate for 5 minutes and colour development was stopped with 2 N H₂SO₄. Plates were read with an Envision at 450 nm wavelength.

### RSV Neutralisation Assay

Sera from immunised mice was analysed for neutralisation activity at the indicated time points. Sera was heat-inactivated and serially diluted in a 96-well plate. Diluted sera were then incubated with the indicated virus at an MOI of 0.04 for 1 hr at 37° C. 20,000 Hep2 cells were then added to the virus/sera mixture and the plates were incubated for 5 days at 37° C. To visualise infection, cells were fixed in acetone. Cells were then washed and stained with biotinylated mAB 133-1H. Cells were then washed and incubated with a strep-HRP antibody. Plates were washed and incubated with TMB substrate for 7 minutes and colour development was stopped with 2 N H₂SO₄. Plates were read with an Envision at 450 nm wavelength.

### Results

### DS2 Immunogens Elicit Enhanced Neutralisation Activity

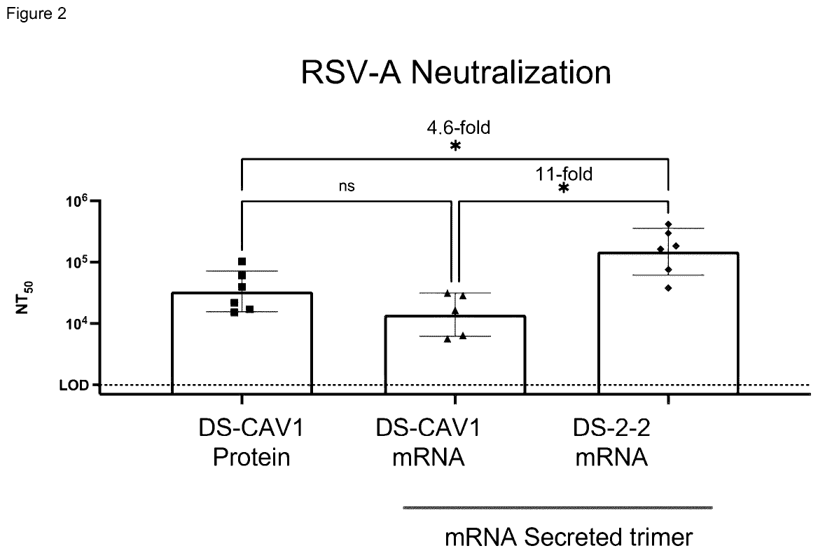

The stabilising mutations found in DS-CAV1 have previously demonstrated that immunogenicity could be greatly improved, as compared to immunisation with wild-type Fusion protein. We next wanted to determine if further stabilisation of the Fusion protein could improve immunogenicity. To test this, we immunised mice with mRNA encoding DS-CAV1 trimer or the DS2-2 immunogen expressed as a trimer, which has been described previously. As a control, mice were also immunised with a prefusion stabilized construct or with recombinant DS-CAV1 protein plus sigma adjuvant system (SAS). Mice immunised with DS2-2 elicited the best RSV-A neutralizing activity, with an 11-fold increase over DS-CAV1 mRNA (p-value <0.05) (FIG. 2). Therefore, we next sought to evaluate the DS2-2 displayed on our panel of nanoparticle scaffolds, to determine if immunogenicity could be further increased.

### DS2 Display on Lumazine Synthase Improves Neutralization Activity

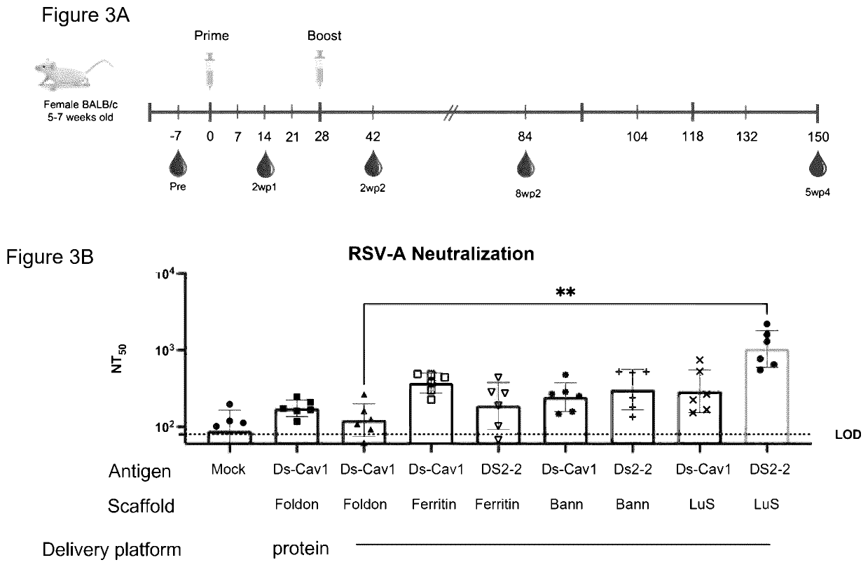

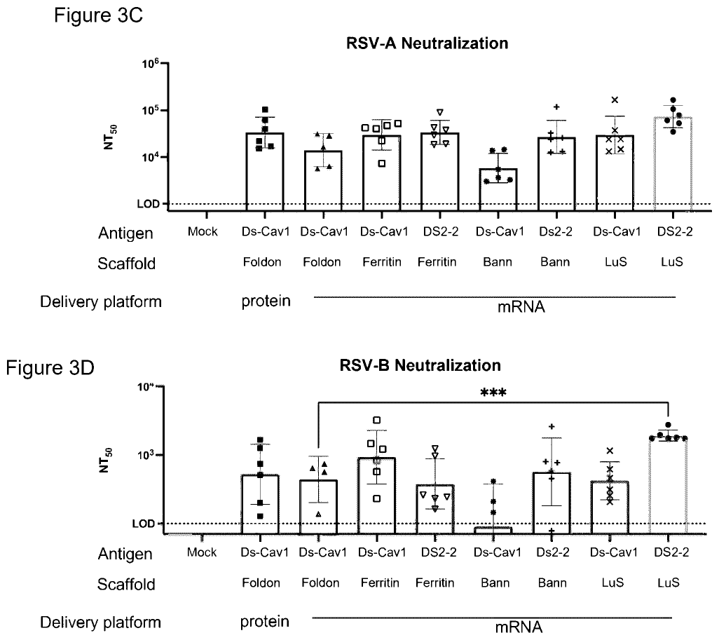

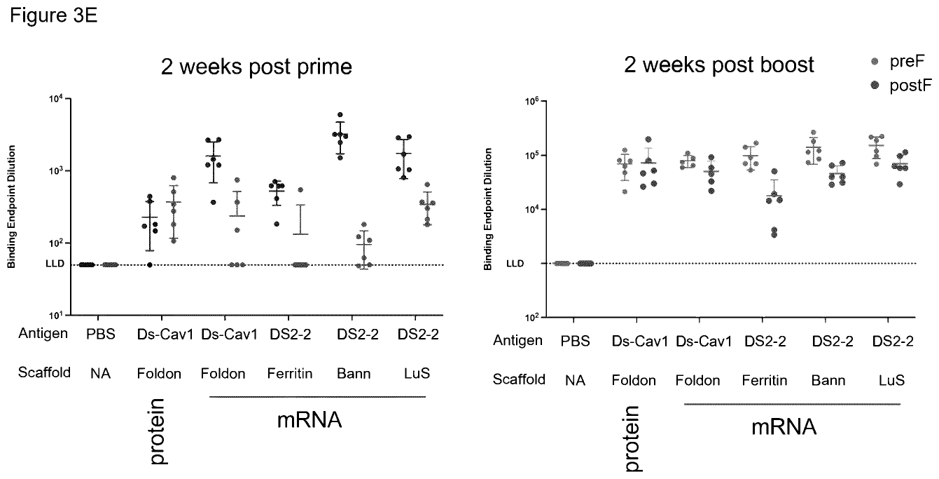

mRNA vaccines were generated encoding the DS2-2 immunogen displayed on ferritin, LuS or Bann VLPs. To evaluate if the additional stabilising modification present in DS2-2, along with the nanoparticle scaffold, could improve immunogenicity, mice were immunised with 2 μg of mRNA on day 0 and 28. On day 14 after the first immunisation, DS2-2-LuS elicited a significant increase in RSV-A neutralisation activity as compared to the DS-Cav1-foldon (trimer) immunised mice (FIG. 3B). By day 42 (2 weeks after the boost), all groups had similar RSV-A neutralisation titres, with DS2-2-LuS trending towards the highest activity (FIG. 3C). However, mice immunised with mRNA-DS2-2-LuS elicited a significant increase in RSV-B neutralization activity, as compared to mRNA-DS-Cav1-foldon (FIG. 3D). Additionally, DS2-2 displayed on LuS elicited superior neutralization activity as compared to DS2-2 display on ferritin or Bann (FIG. 3D).

The ELISA binding antibody results showed a similar response as the neutralisation activity (FIG. 3E). Mice immunised with mRNA-nanoparticles elicited a favourable prefusion response at day 14. However, the prefusion-to-postfusion response was similar at day 42 (FIG. 3E).

### Example 3: Immunization with DS2-2-LuS in RSV Experienced Animals Enhances Neutralization Activity

### Methods

### RSV Infections in Mice

BalB/c mice aged 5-7 weeks were infected with RSV-A by intranasal inoculation. Mice were infected with 10⁴ PFU of virus in a 50 μl volume. Mice were monitored throughout the study.

### Mouse Immunizations

Mouse studies were approved by the Institutional Animal Care and Use Committee at AstraZeneca. Groups of 6 female BalB/c mice aged 5-7 weeks were immunised with the mRNA vaccines. LNP formulations were diluted in PBS according to the indicated dose. Mice were immunised subcutaneously with the diluted LNP in a 50 μl volume on day 80. Mice were bled 2 weeks later to evaluate the immune response.

### Binding ELISAs

ELISAs were performed to evaluate prefusion and postfusion antibody binding titres in immunised mice. 384-well plates were coated with 3 μg/ml of purified recombinant prefusion or postfusion F protein overnight at 4° C. Plates were then washed in PBS-T and blocked in PBS supplemented with 5% milk-powder and 3% BSA for 1 hr at room-temperature. Sera from mice was then diluted in blocking buffer and incubated with the coated plates for 1 hr at room-temperature. Plates were then washed and incubated with an HRP-conjugated anti-mouse IgG secondary antibody for 1 hr. Plates were washed and incubated with TMB substrate for 5 minutes and colour development was stopped with 2 N H₂SO₄. Plates were read with an Envision at 450 nm wavelength.

### RSV Neutralisation Assay

Sera from immunised mice was analysed for neutralisation activity at the indicated time points. Sera was heat-inactivated and serially diluted in a 96-well plate. Diluted sera were then incubated with the indicated virus at an MOI of 0.04 for 1 hr at 37° C. 20,000 Hep2 cells were then added to the virus/sera mixture and the plates were incubated for 5 days at 37° C. To visualise infection, cells were fixed in acetone. Cells were then washed and stained with biotinylated mAB 133-1H. Cells were then washed and incubated with a strep-HRP antibody. Plates were washed and incubated with TMB substrate for 7 minutes and colour development was stopped with 2 N H₂SO₄. Plates were read with an Envision at 450 nm wavelength.

### Results

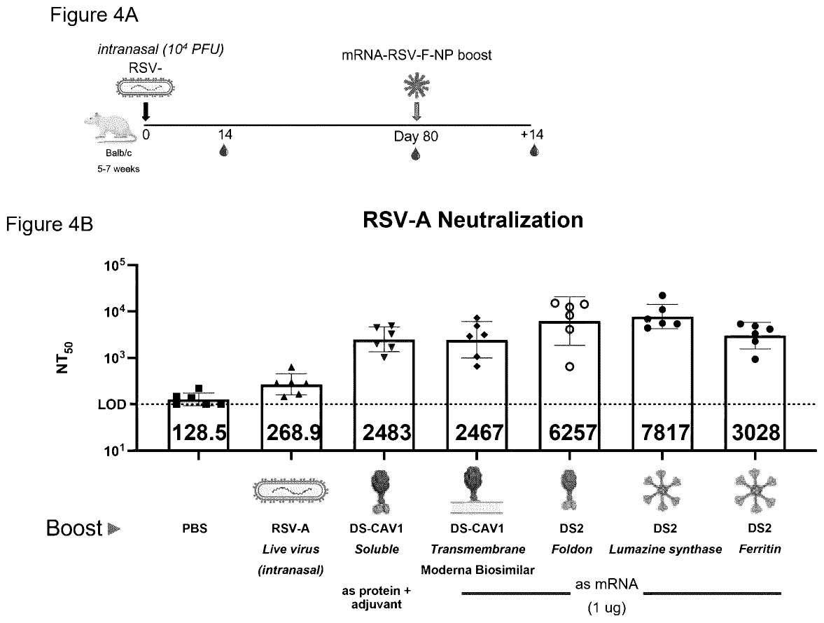

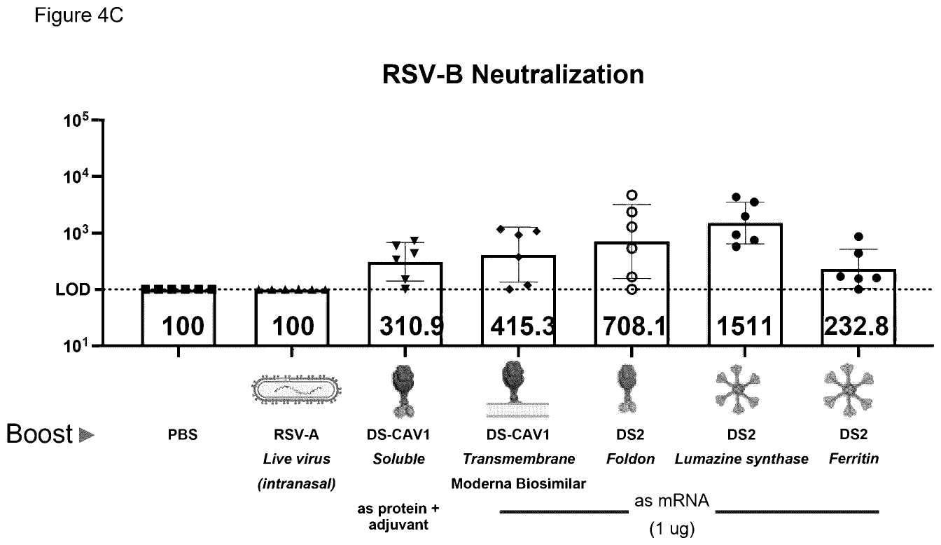

To mimic what is likely to occur in patients, we evaluated our mRNA-protein nanoparticle vaccines in mice that had been previously exposed to RSV infection. Mice were infected by intranasal inoculation with RSV-A (10⁴ PFU) on day 0 and immunised with our vaccine constructs on day 80 (FIG. 4A). DS2-2-Bann was not included due to the poor activity measured in FIG. 3. A control mRNA expressing a transmembrane-bound DS-Cav1 was also included as a benchmark control. Sera was evaluated 2 weeks after immunisation and a 3-fold increase in RSV-A neutralisation activity was observed for DS2-2-LuS relative to the transmembrane-bound DS-Cav1 group (FIG. 4B). Similarly, a 3.6-fold increase in RSV-B neutralisation was observed in mice immunised with DS2-2-LuS.

### Example 4: Modification of the 5′ UTR can Affect eGFP mRNA Expression in A549 Cells and Hela Cells

Here we describe 5′ UTR and 3′ UTR sequences that enhance expression of coding sequences, including in the context of mRNA vaccine vectors.

### Methods

### UTR Cloning

5′ UTR candidate sequences (shown in Table 4 below) were cloned with eGFP and a reference 3′ UTR sequence (albumin, as used in a CureVac vector described, for example, in EP2831240). The cloning was either done using restriction sites (RE) or seamlessly. The 5′ end of the 5′ UTR contained the T7 promoter sequence.

### Generation of In Vitro Transcription (IVT) Template Using PCR

The templates for IVT were generated by PCR using Phusion PCR mix (NEB). The upstream primer contained the T7 promoter sequence and the downstream primer contained the reverse complement of the end of the 3′ UTR of the respective clone, along with a T80 sequence. The resulting PCR product contained a sequence encoding the relevant sequence elements in the following order: T7 promoter-5′ UTR-eGFP-3′ UTR-A80. The PCR reaction was then treated with DpnI to digest template DNA and purified using a PCR purification kit.

mRNA Synthesis Using In Vitro Transcription (IVT)

The templates generated for IVT using PCR were used to prepare mRNA using NEB IVT kits that use the T7 RNA polymerase. The protocol was modified to include CleanCap® (AG). The T7 polymerase incorporates CleanCap® (AG) at the start of each mRNA, giving a Cap1 structure at the 5′ end. The mRNA was either produced using unmodified nucleotides or using modified uridine (5′methoxy uridine) in a 25% ratio with unmodified uridine. After the reaction completed, the DNA-template was digested using RNase free DNase. mRNA transcripts contained a 5′ Cap1-5′ UTR-eGFP coding sequence-3′ UTR-A80 structure. mRNA was then purified using silica columns and resuspended in water.

### Cell Transfection

Lung A549 cells were grown in T175 flasks in A549 complete medium (Ham's F-12K supplemented with 10% FBS) at 37° C. 5% CO₂. Cells were harvested using accutase for 5 min at 37° C., then counted, washed and replated. 100,000 cells in 100 μl media were seeded in each well (96 W plates) the day before. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then the mRNA and Lipofectamine were mixed by vortexing and spun down before incubating for 5-10 min at RT. 10 μl of mRNA/Lipofectamine mix was added to each well.

HeLa cells were grown in T175 flasks in HeLa complete medium (Minimum Essential Medium MEM, supplemented with 10% FBS and 1% non-essential amino acids) at 37° C., 5% CO2. Cells were harvested using accutase for 5 min at 37° C., then counted, washed and replated in collagen-treated plates. 100,000 cells in 100 μl media were seeded in each well (96 W plates, collagen-treated) the day before transfection. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then the mRNA and Lipofectamine were mixed by vortexing and spun down before incubating for 5-10 min at RT. 10 μl of mRNA/Lipofectamine mix was added to each well.

Quantification of eGFP Expression

eGFP fluorescence was detected using an incucyte machine that captures images from the live cells. The fluorescence was measured from the images using Incucyte software in relative fluorescence units (RFU). The data reported in the Figures shows eGFP fluorescence 24 h post-transfection.

### Results

Table 4 shows expression levels by the candidate 5′-UTRs.

TABLE 4

| | | eGFP expression fold- | eGFP expression fold- |
|---|---|---|---|
| ID | | increase vs control | increase vs control |
| Number | 5′-UTR | in A549 cells | in HeLa cells |
| 20 | PRKACB | 4 | 13 |
| 21 | GOT1 | 4 | 13 |
| 15 | Modified (m) | 3 | 7 |
| | CHIT1 | | |
| 22 | GUSB | 3 | 14 |

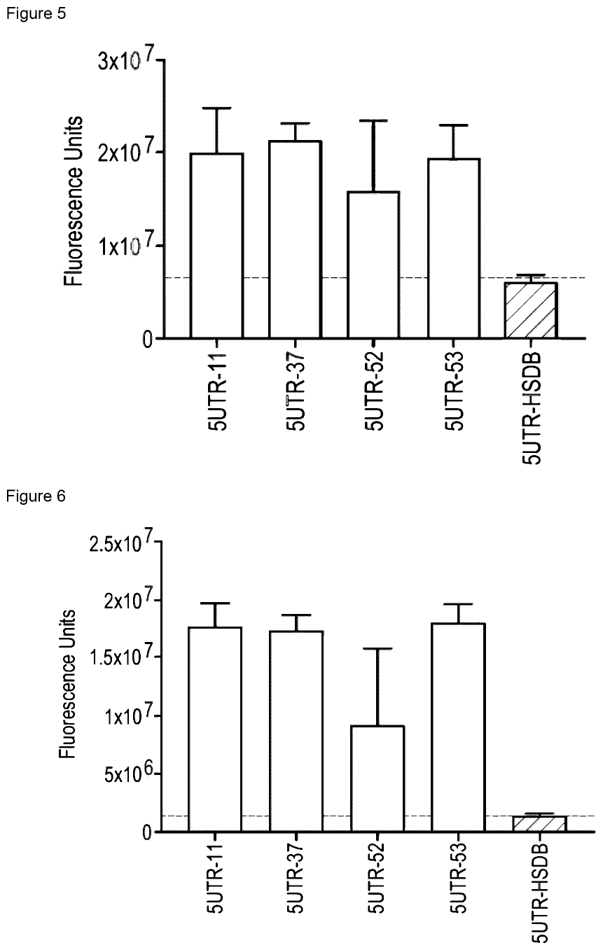

The inclusion of a candidate 5′ UTR (Table 4) was shown to increase the expression of eGFP encoding mRNAs in A549 cells, when compared to eGFP expression with both a control 5′ UTR (HSD17B4) and control 3′ UTR (albumin) (FIG. 5). Inclusion of the GOT1, PRKACB, CHIT1 or GUSB 5′ UTRs corresponded to a 4, 3, 3 and 3-fold increase in eGFP expression, respectively, when compared to expression with a control 5′ UTR (HSD17B4).

This result was substantiated in HeLa cells, in which replacement of the control 5′ UTR with a candidate 5′ UTR (Table 4) was shown to increase eGFP-encoding mRNA expression (FIG. 6). Inclusion of the GOT1, PRKACB, CHIT1 or GUSB 5′ UTRs corresponded to a 13, 13, 7 and 14-fold increase in eGFP expression, respectively, when compared to expression with a control 5′ UTR (HSD17B4).

### Example 5: Modification of the 3′ UTR can Affect eGFP mRNA Expression in A549 Cells and Hela Cells

### Methods

### UTR Cloning

3′ UTR candidate sequences (shown in Table 5 below) were cloned with eGFP and a reference 5′ UTR sequence (HSD17B4, as used in a CureVac vector, described, for example, in EP2831240). The cloning was either done using restriction sites (RE) or seamlessly. The 5′ end of the 5′ UTR contained the T7 promoter sequence.

### Generation of In Vitro Transcription (IVT) Template Using PCR

The templates for IVT were generated by PCR using Phusion PCR mix (NEB). The upstream primer contained the T7 promoter sequence and the downstream primer contained the reverse complement of the end of the 3′ UTR of the respective clone, along with a T80 sequence. The resulting PCR product contained a sequence encoding the relevant sequence elements in the following order: T7 promoter-5′ UTR-eGFP-3′ UTR-A80. The PCR reaction was then treated with DpnI to digest template DNA and purified using a PCR purification kit.

mRNA Synthesis Using In Vitro Transcription (IVT)

The templates generated for IVT using PCR were used to prepare mRNA using NEB IVT kits that use the T7 RNA polymerase. The protocol was modified to include CleanCap® (AG). The T7 polymerase incorporates CleanCap® (AG) at the start of each mRNA, giving a Cap1 structure at the 5′ end. The mRNA was either produced using unmodified nucleotides or using modified uridine (5′methoxy uridine) in a 25% ratio with unmodified uridine. After the reaction completed, the DNA-template was digested using RNase free DNase. mRNA transcripts contained a 5′ Cap1-5′ UTR-eGFP-3′ UTR-A80 structure. mRNA was then purified using silica columns and resuspended in water.

### Cell Transfection

Lung A549 cells were grown in T175 flasks in A549 complete medium (Ham's F-12K supplemented with 10% FBS) at 37° C., 5% CO₂. Cells were harvested using accutase for 5 min at 37° C., then counted, washed and replated. 100,000 cells in 100 μl media were seeded in each well (96 W plates) the day before. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then the mRNA and Lipofectamine were mixed by vortexing and spun down before incubating for 5-10 min at RT. 10 μl of mRNA/Lipofectamine mix was added to each well.

HeLa cells were grown in T175 flasks in HeLa complete medium (Minimum Essential Medium MEM, supplemented with 10% FBS and 1% non-essential amino acids) at 37° C., 5% CO₂. Cells were harvested using accutase for 5 min at 37° C., then counted, washed and replated in collagen-treated plates. 100,000 cells in 100 μl media were seeded in each well (96 W plates, collagen-treated) the day before transfection. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then mRNA and Lipofectamine were mixed by vortexing and spun down before incubating for 5-10 min at RT. 10 μl of mRNA/Lipofectamine mix was added to each well.

Quantification of eGFP Expression

eGFP fluorescence was detected using an incucyte machine that captures images from the live cells. The fluorescence was measured from the images using Incucyte software in relative fluorescence units (RFU). The data reported in the Figures shows eGFP fluorescence 24 h post-transfection.

### Results

Table 5 shows expression levels by the candidate 3′-UTRs.

TABLE 5

| | | eGFP expression fold- | eGFP expression fold- |
|---|---|---|---|
| ID | | increase vs control | increase vs control |
| Number | 3′ UTR | in A549 cells | in HeLa cells |
| 24 | CHIT1 | 3 | 7 |
| 16 | CS | 3 | 4 |
| 25 | PKLR | 2 | 3 |

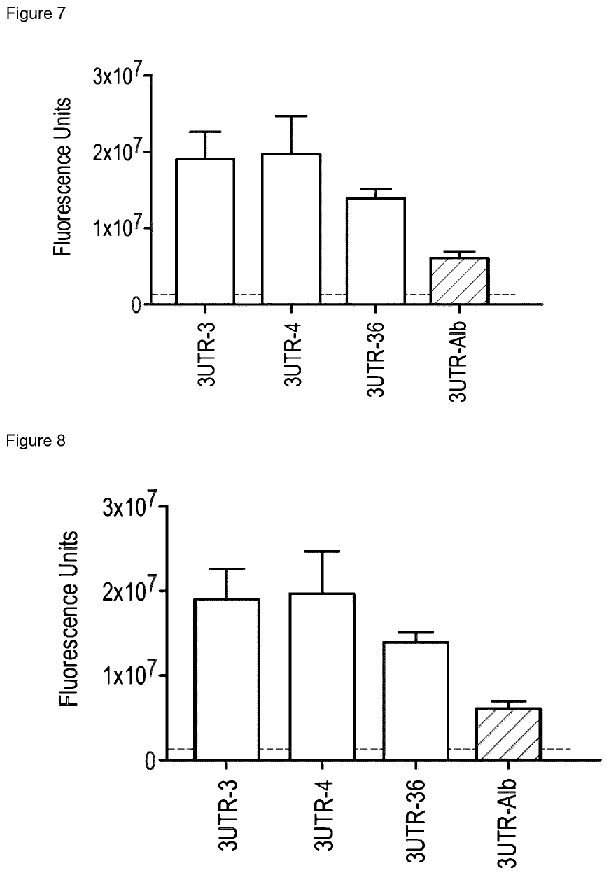

The addition of a candidate 3′ UTR (Table 5) was shown to increase the expression of eGFP encoding mRNAs in A549 cells, when compared to eGFP expression with both a control 3′ UTR (albumin) and control 5′ UTR (HSD17B4) (FIG. 7). The addition of the CHIT1, CS or PKLR 3′-UTR resulted in a 3, 3, and 2-fold increase in eGFP expression, respectively, when compared to expression with a control 3′ UTR (albumin).

This result was replicated in HeLa cells, in which replacement of the control 3′ UTR (albumin) with a candidate 3′ UTR (Table 5) was shown to increase eGFP expression (FIG. 8). As such, addition of the, CHIT1, CS or PKLR 3′ UTRs corresponded with a 7, 4 and 3-fold increase in eGFP expression, respectively, when compared to expression with a control 3′ UTR (albumin).

### Example 6: Combining a Modified 5′ UTR with a Modified 3′ UTR can Increase eGFP mRNA Expression

Combinations of selected 5′ UTRs (Table 4) along with selected 3′ UTRs (Table 5) were assessed, to determine whether a combinatory approach could further increase eGFP mRNA expression. The following combinations were assayed: GOT1/CHIT1, GOT1/CS, PRKACB/CHIT1, PRKACB/CS, CHIT1/CHIT1 and CHIT1/CS.

### Methods

### UTR Cloning

The 5′ UTRs and 3′ UTRs were cloned with eGFP as the ORE. The cloning was either done using restriction sites (RE) or seamlessly. The 5′ end of the 5′ UTR contained the T7 promoter sequence.

### Generation of In Vitro Transcription (IVT) Template Using PCR

The templates for IVT were generated by PCR using Phusion PCR mix (NEB). The upstream primer contained the T7 promoter sequence and the downstream primer contained the reverse complement of the end of the 3′ UTR of the respective clone, along with a T80 sequence. The resulting PCR product contained a sequence encoding the relevant sequence elements in the following order: T7 promoter-5′ UTR-ORF-3′ UTR-A80. The PCR reaction was then treated with DpnI to digest template DNA and purified using a PCR purification kit.

mRNA Synthesis Using In Vitro Transcription (IVT)

The templates generated for IVT using PCR were used to prepare mRNA using NEB IVT kits that use the T7 RNA polymerase. The protocol was modified to include CleanCap® (AG). The T7 polymerase incorporates CleanCap® (AG) at the start of each mRNA, giving a Cap1 structure at the 5′ end. The mRNA was either produced using unmodified nucleotides or using modified uridine (5′methoxy uridine) in a 25% ratio with unmodified uridine. After the reaction completed, the DNA template was digested using RNase free DNase. mRNA transcripts contained a 5′ Cap-1-5′ UTR-ORF-3′ UTR-A80 structure. mRNA was then purified using silica columns and resuspended in water.

### Cell Transfection

Lung A549 cells were grown in T175 flasks in A549 complete medium (Ham's F-12K supplemented with 10% FBS) at 37° C. 5% CO₂. Cells were harvested using accutase for 5 min at 37° C., then counted, washed and replated. 100,000 cells in 100 μl media were seeded in each well (96 W plates) the day before. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then the mRNA and Lipofectamine were mixed by vortexing and spun down before incubating for 5-10 min at RT. 10 μl of mRNA/Lipofectamine mix was added to each well.

Quantification of eGFP Expression

eGFP fluorescence was detected using an incucyte machine that captures images from the live cells. The fluorescence was measured from the images using Incucyte software in relative fluorescence units (RFU). The data reported in the Figures shows eGFP fluorescence 24 h post-transfection.

### Results

It was previously shown that the incorporation of selected 5′ UTRs and 3′ UTRs with control 3′ UTRs and 5′ UTRs, respectively, could increase the expression of an eGFP (see Examples 3 and 4).

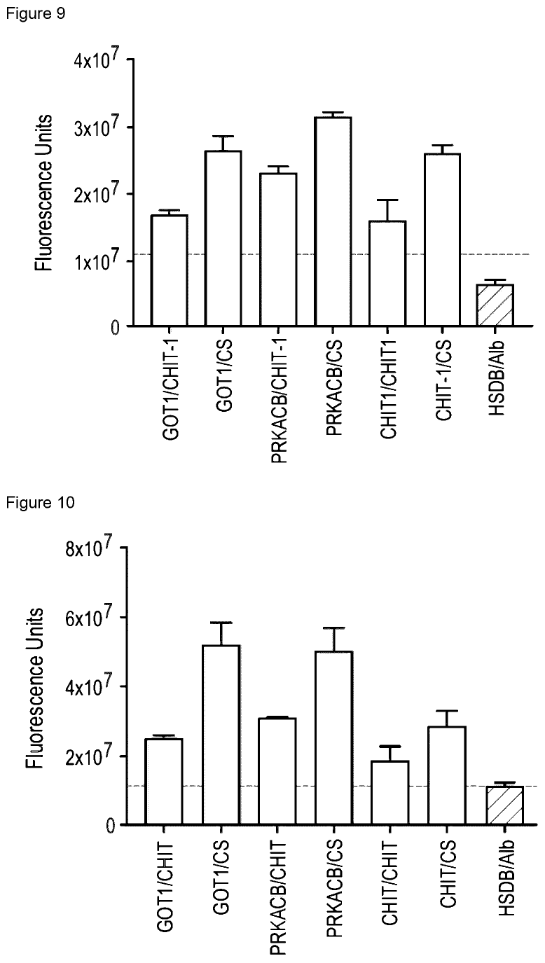

The eGFP mRNA expression with combinations of these 5′ UTRs and 3′ UTRs were compared to expression with a control 5′ UTR (HSD17B4) and a control 3′ UTR (albumin). In all instances, eGFP mRNA expression with a combination of a candidate 5′ UTR and a candidate 3′ UTR was increased, in comparison to control in A549 cells (FIG. 9 and FIG. 10). Interestingly, the general trend for each combination was conserved irrespective of whether the mRNA comprised modified base (FIG. 10) or not (FIG. 9). In both instances, the GOT/CS, PRKACB/CS and CHIT/CS resulted in the greatest increase in GFP fluorescence compared to the control mRNA (HSDB/Alb). Surprisingly, GOT/CS1 appeared to work best in a modified mRNA context (FIG. 10), whereas PRKACB/CS resulted in highest GFP fluorescence levels when wild-type bases were used.

### Example 7: Combining a Modified 5′ UTR with a Modified 3′ UTR can Increase the Expression of scFv-Fc-Encoding mRNA

In order to study whether the combination of UTRs work as well for increasing expression levels independent of the gene of interest, the combinations were tested for scFv expression. This was again performed in both wild-type mRNA and modified mRNA.

### Methods

### UTR Cloning

The 5′ UTRs and 3′ UTRs were cloned with an scFv-Fc-encoding sequence. The cloning was either done using restriction sites (RE) or seamlessly. The 5′ end of the 5′ UTR contained the T7 promoter sequence.

### Generation of In Vitro Transcription (IVT) Template Using PCR

The templates for IVT were generated by PCR using Phusion PCR mix (NEB). The upstream primer contained the T7 promoter sequence and the downstream primer contained the reverse complement of the end of the 3′ UTR of the respective clone, along with a T80 sequence. The resulting PCR product contained a sequence encoding the relevant sequence elements in the following order: T7 promoter-5′ UTR-ORF-3′ UTR-A80. The PCR reaction was then treated with DpnI to digest template DNA and purified using a PCR purification kit.

mRNA Synthesis Using In Vitro Transcription (IVT)

The templates generated for IVT using PCR were used to prepare mRNA using NEB IVT kits that use the T7 RNA polymerase. The protocol was modified to include CleanCap® (AG). The T7 polymerase incorporates CleanCap® (AG) at the start of each mRNA, giving a Cap1 structure at the 5′ end. The mRNA was either produced using unmodified nucleotide or using modified uridine (5′methoxy uridine) in a 25% ratio with unmodified uridine. After the reaction completed, the DNA-template was digested using RNase free DNase. mRNA transcripts contained a 5′ Cap-1-5′ UTR-ORF-3′ UTR-A80 structure. mRNA was then purified using silica columns and resuspended in water.

### Cell Transfection

Lung A549 cells were grown in T175 flasks in A549 complete medium (Ham's F-12K supplemented with 10% FBS) at 37° C., 5% CO₂. Cells were harvested using accutase for 5 min at 37° C., then counted, washed and replated. 100,000 cells in 100 μl media were seeded in each well (96 W plates) the day before. On the day of transfection, old media was aspirated and 140 μl of fresh media was added to each well. 1 μl of 100 ng/μl mRNA was diluted in 4 μl of OptiMEM, and 0.3 μl Lipofectamine 2000 was diluted in 4.7 μl OptiMEM. Then the mRNA and Lipofectamine were mixed by vortexing and spun down before incubating for 5-10 min at RT. 10 μl of mRNA/Lipofectamine mix was added to each well. 100 μl supernatant was collected at specified timepoints.

scFv-FC Quantification

Cell supernatant (sup) was harvested from the cells transfected with scFv-Fc mRNA after 24 hours. The sups were frozen at −80° C. until quantified. For quantification, cis-bio kit for Fc quantification was used. The principle for quantification is based on a competitive immunoassay using HTRF technology. hFc-tagged proteins (or antibody) can displace the binding between IgG labelled with d2 and PAb anti-human Fc labelled with Cryptate. Specific signal (i.e. energy transfer) is inversely proportional to the concentration of human Fc in the sample or standard. A standard curve from known concentrations of scFv-Fc was produced and the signal from sups of scFv-Fc transfected cells was interpolated using this standard curve to quantify the amount of scFv-Fc present in the sup. The concentration of scFv-Fc was measured in ng/mL. The data shown in the Figures reports scFv-Fc levels 24 h post transfection.

### Results

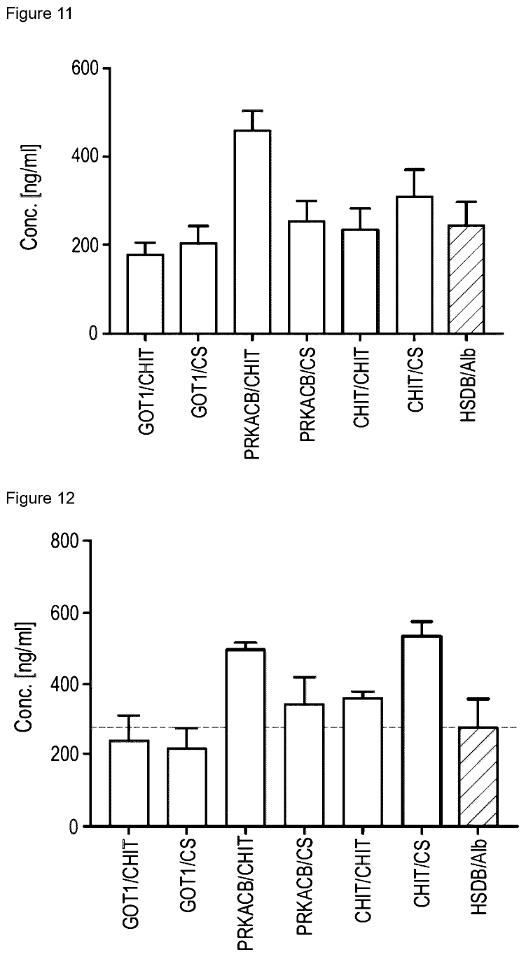

Once again, the scFv mRNA expression with combinations of these 5′ UTRs and 3′ UTRs were compared to expression with a control 5′ UTR (HSD17B4) and a control 3′ UTR (albumin). Interestingly, as with eGFP fluorescence levels in Example 3, the PRKACB/CHIT resulted in the highest expression levels when wild-type mRNA was used (FIG. 11). However, apart from CHIT/CS, most other of the UTR combos did not impact detectable scFv expression levels compared to the control (HSBD/Alb). By contrast, CHIT/CS and PRKACB/CHIT performed best compared to the control when modified mRNA was used as the expression substrate (FIG. 12).

Therefore, out of all novel UTRs tested, whether alone or in combination, the CHIT/CS and PRKACB/CHIT combinations generated the greatest increase in expression levels, agnostic of the gene of interest or whether the mRNA comprised modified or wild-type U.

### Example 8—Enhanced In Vitro Protein Expression from mRNA Vaccine Vectors

Next, one of the optimum UTR combinations (CHIT/CS) was tested head-to-head against mRNA comprising putative UTR pairs from Moderna and Pfizer/BioNTech (mRNA Comp A and mRNA Comp B) using the UTR sequences reported on 14 Apr. 2021 by Andrew Fire and colleagues at Stanford University via GitHub. This time, modified mRNA comprised 100% 5′methoxy uridine (rather than 25%, as in Examples 4-7).

eGFP Construct Cloning

mRNA constructs encoding the eGFP reporter and employing the CHIT1 5′ UTR (SEQ ID NO: 15) paired with a CS 3′ UTR (SEQ ID NO:16) were designed (mRNA_AZ). In parallel, mRNA comparator A and mRNA comparator B constructs were designed to encode eGFP flanked by the putative UTR sequences from each of the COVID vaccines mentioned above. The cloning was performed by Gibson-based assembly methods. Each plasmid possessed a T7 promoter sequence upstream of each 5′ UTR and a polyA track of 80 base pairs downstream of the 3′ UTR with a single BspQI site for subsequent linearisation. All eGFP coding sequences were identical. The full 5′ UTR and 3′ UTR sequences comprising sequences derived from CHIT1 and CS UTRs are set forth in SEQ ID NOs: 43 and 44, respectively.

### Generation of In Vitro Transcription (IVT) Template

The templates for IVT were generated following plasmid purification from bacterial cells in a manner similar to as outlined in Examples 4-7.

mRNA Synthesis Using In Vitro Transcription (IVT)

The template generated for IVT was used to prepare mRNA using NEB IVT kits employing a T7 RNA polymerase. The protocol was modified to include CleanCap® (AG). The T7 polymerase incorporates CleanCap® (AG) at the start of each mRNA, giving a Cap1 structure at the 5′ end. The mRNA was either produced using unmodified nucleotides or using modified uridine (N1-methylpseudouridine) at 100%. Both mRNA comp A and mRNA comp B (the mRNAs comprising the putative UTRs derived from the GitHub database) contained 100% modified U. After the reaction completed, the DNA template was digested using RNase-free DNase. mRNA was then purified using silica columns and resuspended in water.

Quantification of eGFP Expression

Purified mRNA was transfected into either BHK-21 or HEK293 cells with Lipofectamine MessengerMAX transfection reagent (Thermo Fisher) per manufacturer's protocol. eGFP fluorescence was detected using an IncuCyte instrument which captures images from live, RNA-transfected cells over the course of 96 hours. The fluorescence was measured from the images using IncuCyte software as relative fluorescence units (arbitrary units).

### Results

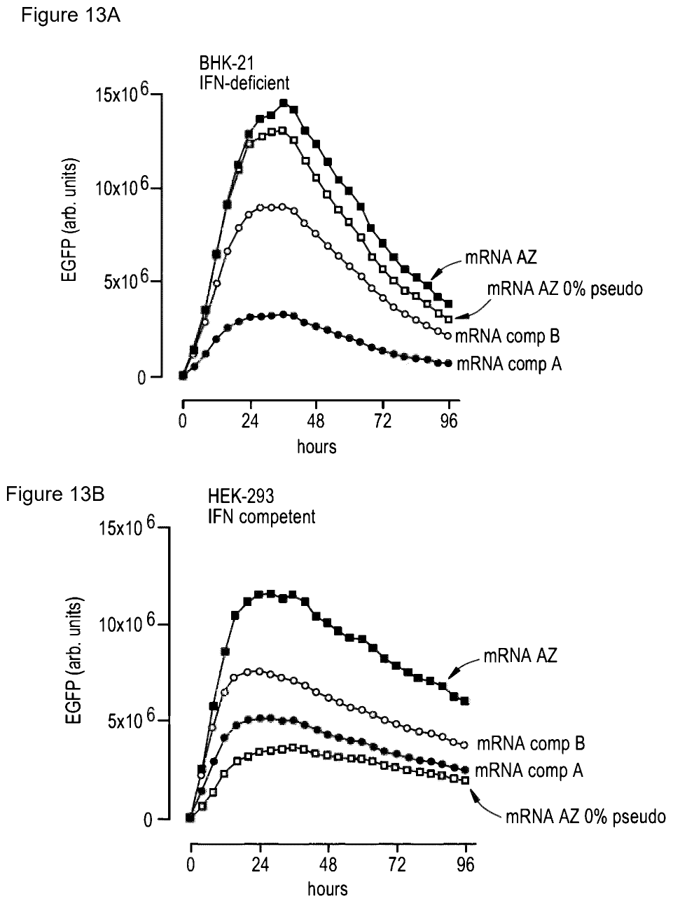

The mRNAs employing the 5′ UTR CHIT1 (SEQ ID NO: 15) and 3′ UTR CS (SEQ ID NO: 16) UTR set generated the highest levels of eGFP expression in both BHK-21 (FIG. 13A) and HEK293 (FIG. 13B) cells compared to two competitor mRNA molecules (mRNA Comp A and mRNA Comp B). Maximal expression from mRNA was dependent on the incorporation of modified nucleotides like N1-Methylpseudouridine (pseudoU) in order to evade host cell anti-viral responses among the HEK293 cells (FIG. 13B).

### Example 9—RSV mRNA-VLP Vaccine Elicits High-Titre Neutralising Antibodies in RSV-Experienced Non-Human Primates

Groups of RSV-experienced non-human primates (NHPs) were immunised with a benchmark RSV F protein vaccine (DS-CAV1+adjuvant) or one of two different RSV mRNA vaccines encoding the prefusion stabilised F protein, and the immunogenicity was evaluated by ELISA binding titres and virus neutralisation assays.

### Methods

mRNA Synthesis Using In Vitro Transcription (IVT)

Sequences encoding membrane DS-CAV1 F protein and the DS-2-2 F protein-nanoparticle subunit were cloned into an mRNA vector encoding the T7 promoter, 5′ and 3′ UTRs, and a polyA tail. The template plasmid was linearised by digesting the pDNA with BSPQ1 (NEB, USA) for 4 h at 50° C. mRNAs were synthesized by in vitro transcription using NEB T7 HighScribe supplemented with N1-methyl-pseudouridine and CleanCap-AG (TriLink). IVT reactions were incubated for 5 hr at 37° C., followed by digestion with RNase-free Dnase. mRNA was then purified using LiCl precipitation. All mRNAs were formulated in a lipid nanoparticle (LNP) in 20 mM Tris/Tris-HCL, pH 7.4, 8% w/v sucrose and then stored at −80° C.

### NHP Immunizations

The animal studies were approved by the Institutional Animal Care and Use Committee (IACUC) at AstraZeneca and conducted at BIOQUAL, Inc. (Rockville, MD). 24 cynomolgus macaques (Macaca fascicularis) were divided into three groups. Individual animals received 1 mL intramuscular (IM) injections of either protein or mRNA vaccine on study day 1. Sera samples were obtained 7 days prior to and 14 days after immunization (study days −7 and 14, respectively) to evaluate baseline and post-immunization antibody levels, respectively.

### Binding ELISAs

ELISAs were performed to evaluate prefusion F antibody binding titres in immunised animals. 384-well plates were coated with 3 μg/ml of purified recombinant prefusion F protein overnight at 4° C. Plates were then washed in PBS-T and blocked in PBS supplemented with 5% milk-powder and 3% BSA for 1 hr at room-temperature. Animal sera were then diluted in blocking buffer and incubated with the coated plates for 1 hr at room-temperature. Plates were then washed and incubated with an HRP-conjugated anti-NHP IgG secondary antibody for 1 hr. Plates were washed and incubated with TMB substrate for 5 minutes and colour development was stopped with 2 N H₂SO₄. Plates were read with an Envision at 450 nm wavelength.

### RSV Neutralisation Assay

Sera from immunised animals were analysed for neutralisation activity at the indicated time points. Sera were heat-inactivated and serially diluted in a 96-well plate. Diluted sera were then incubated with the indicated virus at an MOI of 0.04 for 1 hr at 37° C. 20,000 Hep2 cells were then added to the virus/sera mixture and the plates incubated for 5 days at 37° C. To visualise infection, cells were fixed in acetone, washed, and then stained with biotinylated mAB 133-1H. Cells were washed and incubated with a strep-HRP antibody. Plates were washed and incubated with TMB substrate for 7 minutes and colour development was stopped with 2 N H₂SO₄. Plates were read with an Envision at 450 nm wavelength.

### Results

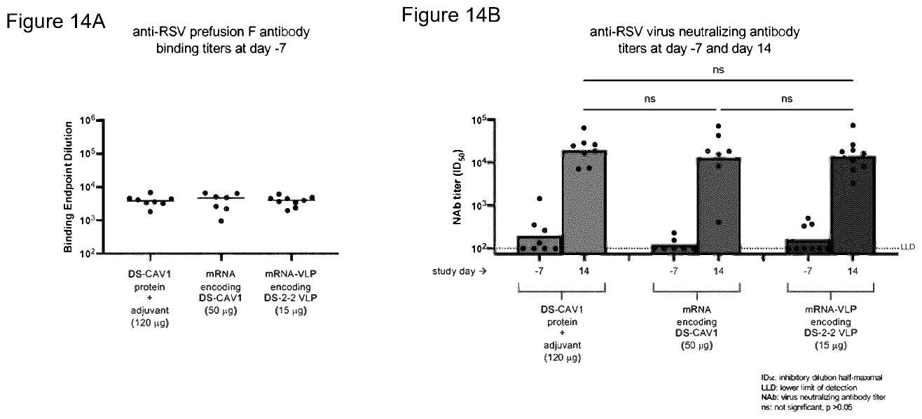

RSV mRNA-VLP expressing the DS-2-2 F protein on the lumazine synthase scaffold elicited a potent virus-neutralizing antibody response comparable to benchmark protein and mRNA vaccines administered at higher doses. We next wanted to compare the boosting capacity of our RSV mRNA-VLP vaccine in RSV-experienced nonhuman primates (NHPs). To test this, we immunised groups of NHPs with either 120 μg of recombinant DS-CAV1 protein+adjuvant, 50 μg of an mRNA vaccine expressing DS-CAV1, or 15 μg of our mRNA-VLP vaccine expressing the DS-2-2 protein fused to the lumazine synthase (LS) scaffold. Prior to study start (day −7) all animals were profiled for baseline anti-RSV prefusion F antibody binding titres (FIG. 14A) and virus neutralizing antibody titres (FIG. 14B) and the groups were found to be balanced. Fourteen days after immunization, all groups of animals had achieved a comparable virus neutralizing antibody titre (FIG. 14B). Importantly, the 15 μg of mRNA-VLP vaccine elicited a very similar response to the protein vaccine and mRNA vaccine which were administered at 120 μg and 50 μg, respectively, which further highlights the immunogenic value of the VLP approach for antigen design and the potential for improved reactogenicity of mRNA-based vaccine approaches.

### Example 10—RSV mRNA-VLP Vaccine Protects Infected Cotton Rats from Virus Replication in the Lungs and Nasal Tissue

Groups of naïve cotton rats were immunised with a benchmark RSV F protein vaccine (DS-CAV1+adjuvant) or one of two different RSV mRNA vaccines encoding the prefusion stabilised F protein to evaluate vaccine efficacy in a virus challenge model.

### Methods

mRNA Synthesis Using In Vitro Transcription (IVT)

Sequences encoding membrane DS-CAV1 F protein and the DS-2-2 F protein-nanoparticle subunit were cloned into an mRNA vector encoding the T7 promoter, 5′ and 3′ UTRs, and a polyA tail. The template plasmid was linearised by digesting the pDNA with BSPQ1 (NEB, USA) for 4 h at 50° C. mRNAs were synthesized by in vitro transcription using NEB T7 HighScribe supplemented with N1-methyl-pseudouridine and CleanCap-AG (TriLink). IVT reactions were incubated for 5 hr at 37° C., followed by digestion with RNase-free DNase. mRNA was then purified using LiCl precipitation. All mRNAs were formulated in a lipid nanoparticle (LNP) in 20 mM Tris/Tris-HCL, pH 7.4, 8% w/v sucrose and then stored at −80° C.

### Cotton Rat Immunization and Virus Challenge

The animal studies were approved by the Institutional Animal Care and Use Committee (IACUC) at AstraZeneca and conducted at Sigmovir Biosystems, Inc. (Rockville, MD). Four groups (n=8 per group) of cotton rats received two intramuscular (IM) injections of either PBS control, protein vaccine, or mRNA vaccine on study days 1 and 21. Sera samples were obtained 21 days after second immunization (study day 42) and just prior to RSV virus challenge. On study day 42, all animals were administered live RSV intranasally and subsequently euthanized five days later (study day 47) at which point lungs and nasal tissues were harvested to assess virus replication by viral plaque assay.

### RSV Neutralisation Assay

Sera from immunised animals were analysed for neutralisation activity at the indicated time points. Sera were heat-inactivated and serially diluted in a 96-well plate. Diluted sera were then incubated with the indicated virus at an MOI of 0.04 for 1 hr at 37° C. 20,000 Hep2 cells were then added to the virus/sera mixture and the plates incubated for 5 days at 37° C. To visualise infection, cells were fixed in acetone, washed, and then stained with biotinylated mAB 133-1H. Cells were washed and incubated with a strep-HRP antibody. Plates were washed and incubated with TMB substrate for 7 minutes and colour development was stopped with 2 N H₂SO₄. Plates were read with an Envision at 450 nm wavelength.

### Results

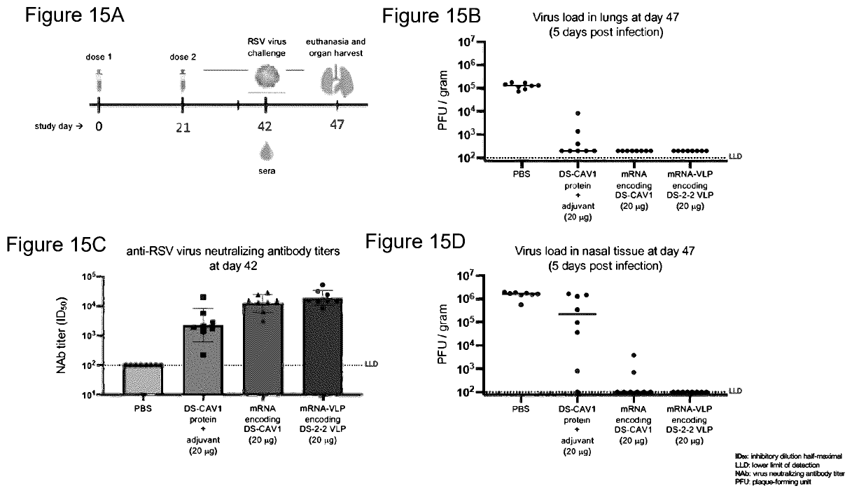

RSV mRNA-VLP expressing the DS-2-2 F protein on the lumazine synthase scaffold elicited a potent virus-neutralizing antibody response and provided complete protection from virus challenge. We next wanted to compare the vaccine efficacy of our RSV mRNA-VLP vaccine in the gold standard cotton rat model of RSV virus challenge. To test this, we immunised groups of cotton rats on study days 1 and 21 with either control PBS, 20 μg of recombinant DS-CAV1 protein+adjuvant, 20 μg of an mRNA vaccine expressing DS-CAV1, or 20 μg of our mRNA-VLP vaccine expressing the DS-2-2 protein fused to the lumazine synthase (LS) scaffold (FIG. 15A). Prior to live RSV virus challenge on study day 42, all groups were profiled for virus-neutralizing antibody titres (FIG. 15A). Both mRNA vaccines groups elicited comparable neutralizing antibody titres which were higher than those measured in the protein vaccine group (FIG. 15B).

Five days after intranasal virus challenge, all groups were euthanized with the lungs and nasal tissues harvested to assess virus replication (FIG. 15A). The levels of virus replication in the lungs—a measure of severe disease—among all vaccinated groups was substantially reduced compared to the PBS control which measured approximately 10⁵ infectious units per gram of lung tissue (FIG. 15B). Interestingly, no detectable virus was recovered in the lungs of mRNA vaccinated groups whereas three animals among the protein vaccine group had detectable levels of virus replication indicating ‘breakthrough’ infection which potentially could be attributed to the lower neutralizing antibodies measured prior to virus challenge (FIG. 15B-15C).

Moreover, a substantial level of virus replication was measured in the nasal cavities of most animals vaccinated with the protein vaccine, corresponding to levels about 10-fold less on average than the PBS control group (FIG. 15D). The group having received the mRNA vaccine expressing DS-CAV-1 had 2 animals demonstrating a ‘breakthrough’ infection. In contrast, there were no ‘breakthrough’ infections among the mRNA-VLP vaccine group suggesting sterilizing immunity. Given that both mRNA vaccines were administered at the same dose, these results taken together further strengthen the value of the VLP antigen design in maximizing the immune response and providing complete protection from virus challenge in the cotton rat model.

### SEQUENCE LISTING

Upper case sequences are protein sequences. Lower case sequences are RNA sequences. The sequences set out the basic amino acid/nucleotide sequences of the elements discussed above. As set out above, the standard nucleotides of the RNA sequences may be replaced with modified versions of the same.

DS2-2
SEQ ID NO: 1
MELLILKANAITTILTAVTFCFASGQNITEEFYQSTCSAVSKGYLGALRTGWYTSVITIE
LSNIKENKCNGTDAKVKLIKQELDKYKNAVTDLQLLMQSTPATGSGSAIASGVAVCK
VLHLEGEVNKIKSALLSTNKAVVSLSGCGVSVLTFKVLDLKNYIDKQLLPILNKQSCSI
PNIETVIEFQQKNNRLLEITREFSVNAGVTTPVSTYMLTNSELLSLINDMPITNDQKKL
MSNNVQIVRQQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVNLCNVDIFNP
KYDCKIMTSKTDVSSSVITSLGAIVSCYGKTKCTASNKCRGIIKTFSNGCDYVSNKGV
DTVSVGNTLYYVNKQEGKSLYVKGEPIINFYDPLVFPSDEFDASISQVNEKINQSLAFI
RKSDELL
DS2-2(A)
SEQ ID NO: 2
MELLILKANAITTILTAVTFCFASGQNITEEFYQSTCSAVSKGYLSALRTGWYTSVITIE
LSNIKENKCNGTDAKVKLIKQELDKYKNAVTELQLLMQSTPATGSGSAIASGVAVCK
VLHLEGEVNKIKSALLSTNKAVVSLSGCGVSVLTFKVLDLKNYIDKQLLPILNKQSCSI
SNIETVIEFQQKNNRLLEITREFSVNAGVTTPVSTYMLTNSELLSLINDMPITNDQKKL
MSNNVQIVRQQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVNLCNVDIFNP
KYDCKIMTSKTDVSSSVITSLGAIVSCYGKTKCTASNKCRGIIKTFSNGCDYVSNKGV
DTVSVGNTLYYVNKQEGKSLYVKGEPIINFYDPLVFPSDEFDASISQVNEKINQSLAFI
RKSDELL
Aquifex aeolicus lumazine synthase
SEQ ID NO: 3
MQIYEGKLTAEGLRFGIVASRFNHALVDRLVEGAIDCIVRHGGREEDITLVRVPGSWEI
PVAAGELARKEDIDAVIAIGVLIRGATPHFDYIASEVSKGLANLSLELRKPITFGVITAD
TLEQAIERAGTKHGNKGWEAALSAIEMANLFKSLR
Linker 1
SEQ ID NO: 4
SGGSSGSSGGS
Linker-Scaffold
SEQ ID NO: 5
SGGSSGSSGGSMQIYEGKLTAEGLRFGIVASRFNHALVDRLVEGAIDCIVRHGGREEDI
TLVRVPGSWEIPVAAGELARKEDIDAVIAIGVLIRGATPHFDYIASEVSKGLANLSLELR
KPITFGVITADTLEQAIERAGTKHGNKGWEAALSAIEMANLFKSLR
DS2-2-LuS
SEQ ID NO: 6
MELLILKANAITTILTAVTFCFASGQNITEEFYQSTCSAVSKGYLGALRTGWYTSVITIE
LSNIKENKCNGTDAKVKLIKQELDKYKNAVTDLQLLMQSTPATGSGSAIASGVAVCK
VLHLEGEVNKIKSALLSTNKAVVSLSGCGVSVLTFKVLDLKNYIDKQLLPILNKQSCSI
PNIETVIEFQQKNNRLLEITREFSVNAGVTTPVSTYMLTNSELLSLINDMPITNDQKKL
MSNNVQIVRQQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVNLCNVDIFNP
KYDCKIMTSKTDVSSSVITSLGAIVSCYGKTKCTASNKCRGIIKTFSNGCDYVSNKGV
DTVSVGNTLYYVNKQEGKSLYVKGEPIINFYDPLVFPSDEFDASISQVNEKINQSLAFI
RKSDELLSGGSSGSSGGSMQIYEGKLTAEGLRFGIVASRFNHALVDRLVEGAIDCIVRH
GGREEDITLVRVPGSWEIPVAAGELARKEDIDAVIAIGVLIRGATPHFDYIASEVSKGLA
NLSLELRKPITFGVITADTLEQAIERAGTKHGNKGWEAALSAIEMANLFKSLR
DS2-2(A)-LuS
SEQ ID NO: 7
MELLILKANAITTILTAVTFCFASGQNITEEFYQSTCSAVSKGYLSALRTGWYTSVITIE
LSNIKENKCNGTDAKVKLIKQELDKYKNAVTELQLLMQSTPATGSGSAIASGVAVCK
VLHLEGEVNKIKSALLSTNKAVVSLSGCGVSVLTFKVLDLKNYIDKQLLPILNKQSCSI
SNIETVIEFQQKNNRLLEITREFSVNAGVTTPVSTYMLTNSELLSLINDMPITNDQKKL
MSNNVQIVRQQSYSIMCIIKEEVLAYVVQLPLYGVIDTPCWKLHTSPLCTTNTKEGSN
ICLTRTDRGWYCDNAGSVSFFPQAETCKVQSNRVFCDTMNSRTLPSEVNLCNVDIFNP
KYDCKIMTSKTDVSSSVITSLGAIVSCYGKTKCTASNKCRGIIKTFSNGCDYVSNKGV
DTVSVGNTLYYVNKQEGKSLYVKGEPIINFYDPLVFPSDEFDASISQVNEKINQSLAFI
RKSDELLSGGSSGSSGGSMQIYEGKLTAEGLRFGIVASRFNHALVDRLVEGAIDCIVRH
GGREEDITLVRVPGSWEIPVAAGELARKEDIDAVIAIGVLIRGATPHFDYIASEVSKGLA
NLSLELRKPITFGVITADTLEQAIERAGTKHGNKGWEAALSAIEMANLFKSLR
DS2-2
SEQ ID NO: 8
auggaauugcugaucuugaaagccaacgccaucacaaccauauugacagcaguaaccuuuuguuucgcaaguggucaaaa
uauuacugaagaauucuaccaaucaacguguagugcggucucaaagggcuaucuuggcgcccugcgcaccggcugguau
acauccgucauaacgaucgaacugagcaacaucaaagagaacaagugcaaugggacugacgcaaaaguuaagcuuaucaa
acaggagcuggacaaauacaaaaaugcugucacggacuugcagcucuugaugcaaagcacgccggcgaccggcucuggaa
gugcgauugcaucagguguugcagucuguaagguauugcaccucgaaggugaaguuaauaagauaaaguccgcccuucu
uuccacuaacaaggccguagugucuuugagcgguugugggguauccguauugacauuuaaagugcuugaucucaagaac
uacaucgacaaacagcucuugcccauccucaacaaacaaagcugcuccauaccaaacauagaaacugugauugaguuccaa
cagaaaaacaauaggcuccuugaaauaacgcgggaguuuuccguuaaugcggggguaacuacuccaguuagcacguaua
ugcucacuaauagugaacucuugucccucauaaaugauaugccgauuaccaaugaccagaaaaaacucaugagcaauaac
guccagauuguaagacaacaguccuacaguauaauguguauaaucaaggaggaagucuuggcuuaugugguucagcuuc
cacucuauggcgugauagacacacccuguuggaagcuucauacuagcccacucugcacuacaaacacaaaggaggggagu
aauaucugucuuacccgcacagaccgagggugguauugcgauaaugcgggguccguaagcuucuucccccaagcggaga
cuugcaagguacagaguaaccgagucuucugcgauacuaugaauagucgcacacucccaagcgagguaaauuugugcaac
guugauauauuuaaucccaaauacgacuguaagauuaugacuuccaagacagacgugucuaguucugucauuacucu
ucggugcaauuguuuccuguuaugggaagacaaaguguacugcgucaaauaagugucgaggcauaauaaaaacuuucuc
aaauggaugcgacuacgucagcaauaaagggguugacacgguuucaguggguaauacccuuuacuaugucaacaagcaa
gaaggaaagagcuuguaugucaagggagagccaaucaucaauuucuaugacccccuuguauuucccucugaugaguucg
acgcuuccauaagccaggugaaugaaaaaauuaaucaaagucuggcuuucauucggaaaucagacgaacuuuug
DS2-2(A)
SEQ ID NO: 9
auggaauugcugaucuugaaagccaacgccaucacaaccauauugacagcaguaaccuuuuguuucgcaaguggucaaaa
uauuacugaagaauucuaccaaucaacguguagugcggucucaaagggcuaucuuucagcccugcgcaccggcugguau
acauccgucauaacgaucgaacugagcaacaucaaagagaacaagugcaaugggacugacgcaaaaguuaagcuuaucaa
acaggagcuggacaaauacaaaaaugcugucacggaguugcagcucuugaugcaaagcacgccggcgaccggcucuggaa
gugcgauugcaucagguguugcagucuguaagguauugcaccucgaaggugaaguuaauaagauaaaguccgcccuucu
uuccacuaacaaggccguagugucuuugagcgguugugggguauccguauugacauuuaaagugcuugaucucaagaac
uacaucgacaaacagcucuugcccauccucaacaaacaaagcugcuccauauccaacauagaaacugugauugaguuccaa
cagaaaaacaauaggcuccuugaaauaacgcgggaguuuuccguuaaugcggggguaacuacuccaguuagcacguaua
ugcucacuaauagugaacucuugucccucauaaaugauaugccgauuaccaaugaccagaaaaaacucaugagcaauaac
guccagauuguaagacaacaguccuacaguauaauguguauaaucaaggaggaagucuuggcuuaugugguucagcuuc
cacucuauggcgugauagacacacccuguuggaagcuucauacuagcccacucugcacuacaaacacaaaggaggggagu
aauaucugucuuacccgcacagaccgagggugguauugcgauaaugcgggguccguaagcuucuucccccaagcggaga
cuugcaagguacagaguaaccgagucuucugcgauacuaugaauagucgcacacucccaagcgagguaaauuugugcaac
guugauauauuuaaucccaaauacgacuguaagauuaugacuuccaagacagacgugucuaguucugucauuacucu
ucggugcaauuguuuccuguuaugggaagacaaaguguacugcgucaaauaagugucgaggcauaaaaaaacuuucuc
aaauggaugcgacuacgucagcaauaaagggguugacacgguuucaguggguaauacccuuuacuaugucaacaagcaa
gaaggaaagagcuuguaugucaagggagagccaaucaucaauuucuaugacccccuuguauuucccucugaugaguucg
acgcuuccauaagccaggugaaugaaaaaauuaaucaaagucuggcuuucauucggaaaucagacgaacuuuug
Aquifex aeolicus lumazine synthase
SEQ ID NO: 10
augcagauuuaugagggcaagcucacagcagaaggcuugagauucggcauuguagccucucgcuucaaccacgcacucg
uugaccgccuugucgaaggugcuauugacuguaucguucggcaugggggucgcgaggaagauauaacucugguuagag
uuccugguaguugggagauccccguugcggcaggagagcuggcuagaaaagaagacaucgacgcaguaaucgccaucgg
cguauugauucgcggggcuaccccgcauuucgauuacauagcgucugaaguaaguaagggacuggcaaaucucucauug
gaguugcgaaagccgauuacuuuuggagucaucaccgccgacacccuggaacaagccauugaacgggggguaccaagca
ugguaauaagggcugggaagcggcgcucagugccauugaaauggcgaaccuguuuaaaucacugagg
Linker 1
SEQ ID NO: 11
uccggcggaaguucuggcucuagugggggaagc
Linker-Scaffold
SEQ ID NO: 12
uccggcggaaguucuggcucuagugggggaagcaugcagauuuaugagggcaagcucacagcagaaggcuugagauucg
gcauuguagccucucgcuucaaccacgcacucguugaccgccuugucgaaggugcuauugacuguauguuggcaugg
gggucgcgaggaagauauaacucugguuagaguuccugguaguugggagauccccguugcggcaggagagcuggcuag
aaaagaagacaucgacgcaguaaucgccaucggcguauugauucgcggggcuaccccgcauuucgauuacauaggucu
gaaguaaguaagggacuggcaaaucucucauuggaguugcgaaagccgauuacuuuuggagucaucaccgccgacaccc
uggaacaagccauugaacgggggguaccaagcaugguaauaagggcugggaagcggcgcucagugccauugaaauggc
gaaccuguuuaaaucacugagg
DS2-2-LuS
SEQ ID NO: 13
auggaauugcugaucuugaaagccaacgccaucacaaccauauugacagcaguaaccuuuuguuucgcaaguggucaaaa
uauuacugaagaauucuaccaaucaacguguagugcggucucaaagggcuaucuuggcgcccugcgcaccggcugguau
acauccgucauaacgaucgaacugagcaacaucaaagagaacaagugcaaugggacugacgcaaaaguuaagcuuaucaa
acaggagcuggacaaauacaaaaaugcugucacggacuugcagcucuugaugcaaagcacgccggcgaccggcucuggaa
gugcgauugcaucagguguugcagucuguaagguauugcaccucgaaggugaaguuaauaagauaaaguccgcccuucu
uuccacuaacaaggccguagugucuuugagcgguugugggguauccguauugacauuuaaagugcuugaucucaagaac
uacaucgacaaacagcucuugcccauccucaacaaacaaagcugcuccauaccaaacauagaaacugugauugaguuccaa
cagaaaaacaauaggcuccuugaaauaacgcgggaguuuuccguuaaugcggggguaacuacuccaguuagcacguaua
ugcucacuaauagugaacucuugucccucauaaaugauaugccgauuaccaaugaccagaaaaaacucaugagcaauaac
guccagauuguaagacaacaguccuacaguauaauguguauaaucaaggaggaagucuuggcuuaugugguucagcuuc
cacucuauggcgugauagacacacccuguuggaagcuucauacuagcccacucugcacuacaaacacaaaggaggggagu
aauaucugucuuacccgcacagaccgagggugguauugcgauaaugcgggguccguaagcuucuucccccaagcggaga
cuugcaagguacagaguaaccgagucuucugcgauacuaugaauagucgcacacucccaagcgagguaaauuugugcaac
guugauauauuuaaucccaaauacgacuguaagauuaugacuuccaagacagacgugucuaguucugucauuacuucuc
ucggugcaauuguuuccuguuaugggaagacaaaguguacugcgucaaauaagugucgaggcauaauaaaaacuuucuc
aaauggaugcgacuacgucagcaauaaagggguugacacgguuucaguggguaauacccuuuacuaugucaacaagcaa
gaaggaaagagcuuguaugucaagggagagccaaucaucaauuucuaugacccccuuguauuucccucugaugaguucg
acgcuuccauaagccaggugaaugaaaaaauuaaucaaagucuggcuuucauucggaaaucagacgaacuuuuguccggc
ggaaguucuggcucuagugggggaagcaugcagauuuaugagggcaagcucacagcagaaggcuugagauucggcauu
guagccucucgcuucaaccacgcacucguugaccgccuugucgaaggugcuauugacuguauguuggcauggggguc
gcgaggaagauauaacucugguuagaguuccugguaguugggagauccccguugcggcaggagagcuggcuagaaaaga
agacaucgacgcaguaaucgccaucggcguauugauucgcggggcuaccccgcauuucgauuacauagcgucugaagua
aguaagggacuggcaaaucucucauuggaguugcgaaagccgauuacuuuuggagucaucaccgccgacacccuggaac
aagccauugaacgggggguaccaagcaugguaauaagggcugggaagcggcgcucagugccauugaaauggcgaaccu
guuuaaaucacugagg
DS2-2(A)-LuS
SEQ ID NO: 14
auggaauugcugaucuugaaagccaacgccaucacaaccauauugacagcaguaaccuuuuguuucgcaaguggucaaaa
uauuacugaagaauucuaccaaucaacguguagugcggucucaaagggcuaucuuucagcccugcgcaccggcugguau
acauccgucauaacgaucgaacugagcaacaucaaagagaacaagugcaaugggacugacgcaaaaguuaagcuuaucaa
acaggagcuggacaaauacaaaaaugcugucacggaguugcagcucuugaugcaaagcacgccggcgaccggcucuggaa
gugcgauugcaucagguguugcagucuguaagguauugcaccucgaaggugaaguuaauaagauaaaguccgcccuucu
uuccacuaacaaggccguagugucuuugagcgguugugggguauccguauugacauuuaaagugcuugaucucaagaac
uacaucgacaaacagcucuugcccauccucaacaaacaaagcugcuccauauccaacauagaaacugugauugaguuccaa
cagaaaaacaauaggcuccuugaaauaacggggaguuuuccguuaaugcggggguaacuacuccaguuagcacguaua
ugcucacuaauagugaacucuugucccucauaaaugauaugccgauuaccaaugaccagaaaaaacucaugagcaauaac
guccagauuguaagacaacaguccuacaguauaauguguauaaucaaggaggaagucuuggcuuaugugguucagcuuc
cacucuauggcgugauagacacacccuguuggaagcuucauacuagcccacucugcacuacaaacacaaaggaggggagu
aauaucugucuuacccgcacagaccgagggugguauugcgauaaugcgggguccguaagcuucuucccccaagcggaga
cuugcaagguacagaguaaccgagucuucugcgauacuaugaauagucgcacacucccaagcgagguaaauuugugcaac
guugauauauuuaaucccaaauacgacuguaagauuaugacuuccaagacagacgugucuaguucugucauuacuucuc
ucggugcaauuguuuccuguuaugggaagacaaaguguacugcgucaaauaagugucgaggcauaaaaaaacuuucuc
aaauggaugcgacuacgucagcaauaaagggguugacacgguuucaguggguaauacccuuuacuaugucaacaagcaa
gaaggaaagagcuuguaugucaagggagagccaaucaucaauuucuaugacccccuuguauuucccucugaugaguucg
acgcuuccauaagccaggugaaugaaaaaauuaaucaaagucuggcuuucauucggaaaucagacgaacuuuuguccggc
ggaaguucuggcucuagugggggaagcaugcagauuuaugagggcaagcucacagcagaaggcuugagauucggcauu
guagccucucgcuucaaccacgcacucguugaccgccuugucgaaggugcuauugacuguaucguucggcauggggguc
gcgaggaagauauaacucugguuagaguuccugguaguugggagauccccguugcggcaggagagcuggcuagaaaaga
agacaucgacgcaguaaucgccaucggcguauugauucgcggggcuaccccgcauuucgauuacauagcgucugaagua
aguaagggacuggcaaaucucucauuggaguugcgaaagccgauuacuuuuggagucaucaccgccgacacccuggaac
aagccauugaacgggggguaccaagcaugguaauaagggcugggaagcggcgcucagugccauugaaauggcgaaccu
guuuaaaucacugagg
modified (m) CHIT1 5′ UTR
SEQ ID NO: 15
auugugcugcauc
CS 3′ UTR
SEQ ID NO: 16
aacuggagacugggugaaagugacuaccagaaagugaggaagccuaaauaaa
DS2-2-LuS Complete mRNA
SEQ ID NO: 17
aggauugugcugcaucaagcuugccgccaccauggaauugcugaucuugaaagccaacgccaucacaaccauauugacag
caguaaccuuuuguuucgcaaguggucaaaauauuacugaagaauucuaccaaucaacguguagugcggucucaaaggg
cuaucuuggcgcccugcgcaccggcugguauacauccgucauaacgaucgaacugagcaacaucaaagagaacaagugca
augggacugacgcaaaaguuaagcuuaucaaacaggagcuggacaaauacaaaaaugcugucacggacuugcagcucuug
augcaaagcacgccggcgaccggcucuggaagugcgauugcaucagguguugcagucuguaagguauugcaccucgaag
gugaaguuaauaagauaaaguccgcccuucuuuccacuaacaaggccguagugucuuugagcgguugugggguauccgu
auugacauuuaaagugcuugaucucaagaacuacaucgacaaacagcucuugcccauccucaacaaacaaagcugcucca
uaccaaacauagaaacugugauugaguuccaacagaaaaacaauaggcuccuugaaauaacggggaguuuuccguuaau
gcggggguaacuacuccaguuagcacguauaugcucacuaauagugaacucuugucccucauaaaugauaugccgauua
ccaaugaccagaaaaaacucaugagcaauaacguccagauuguaagacaacaguccuacaguauaauguguauaaucaag
gaggaagucuuggcuuaugugguucagcuuccacucuauggcgugauagacacacccuguuggaagcuucauacuagcc
cacucugcacuacaaacacaaaggaggggaguaauaucugucuuacccgcacagaccgagggugguauugcgauaaugcg
ggguccguaagcuucuucccccaagcggagacuugcaagguacagaguaaccgagucuucugcgauacuaugaauaguc
gcacacucccaagcgagguaaauuugugcaacguugauauauuuaaucccaaauacgacuguaagauuaugacuuccaag
acagacgugucuaguucugucauuacuucucucggugcaauuguuuccuguuaugggaagacaaguguacuggucaa
auaagugucgaggcauaauaaaaacuuucucaaauggaugcgacuacgucagcaauaaagggguugacacgguuucagu
ggguaauacccuuuacuaugucaacaagcaagaaggaaagagcuuguaugucaagggagagccaaucaucaauuucuau
gacccccuuguauuucccucugaugaguucgacgcuuccauaagccaggugaaugaaaaaauuaaucaaagucuggcuu
ucauucggaaaucagacgaacuuuuguccggcggaaguucuggcucuagugggggaagcaugcagauuuaugagggcaa
gcucacagcagaaggcuugagauucggcauuguagccucucgcuucaaccacgcacucguugaccgccuugucgaaggu
gcuauugacuguaucguucggcaugggggucgcgaggaagauauaacucugguuagaguuccugguaguugggagauc
cccguugcggcaggagagcuggcuagaaaagaagacaucgacgcaguaaucgccaucggcguauugauucgcggggcua
ccccgcauuucgauuacauagcgucugaaguaaguaagggacuggcaaaucucucauuggaguugcgaaagccgauuac
uuuuggagucaucaccgccgacacccuggaacaagccauugaacgggggguaccaagcaugguaauaagggcugggaa
gcggcgcucagugccauugaaauggcgaaccuguuuaaaucacugaggugaugauaauaggacuaguggauccaacugg
agacugggugaaagugacuaccagaaagugaggaagccuaaauaaaccuagcguacguaaaaaauggaaagaaccuagcg
uacgaaaaaaaaaaaaaaaaaaaaaaaaaaaaaaaaaaaaaaaaaaaaaaaaaaaaaaaaaaaaaaaaaaaaaaaaaaaaa
DS2-2(A)-LuS Complete mRNA
SEQ ID NO: 18
aggauugugcugcaucaagcuugccgccaccauggaauugcugaucuugaaagccaacgccaucacaaccauauugacag
caguaaccuuuuguuucgcaaguggucaaaauauuacugaagaauucuaccaaucaacguguagugcggucucaaaggg
cuaucuuucagcccugcgcaccggcugguauacauccgucauaacgaucgaacugagcaacaucaaagagaacaagugca
augggacugacgcaaaaguuaagcuuaucaaacaggagcuggacaaauacaaaaaugcugucacggaguugcagcucuug
augcaaagcacgccggcgaccggcucuggaagugcgauugcaucagguguugcagucuguaagguauugcaccucgaag
gugaaguuaauaagauaaaguccgcccuucuuuccacuaacaaggccguagugucuuugaggguugugggguauccgu
auugacauuuaaagugcuugaucucaagaacuacaucgacaaacagcucuugcccauccucaacaaacaaagcugcucca
uauccaacauagaaacugugauugaguuccaacagaaaaacaauaggcuccuugaaauaacggggaguuuuccguuaau
gcggggguaacuacuccaguuagcacguauaugcucacuaauagugaacucuugucccucauaaaugauaugccgauua
ccaaugaccagaaaaaacucaugagcaauaacguccagauuguaagacaacaguccuacaguauaauguguauaaucaag
gaggaagucuuggcuuaugugguucagcuuccacucuauggcgugauagacacacccuguuggaagcuucauacuagcc
cacucugcacuacaaacacaaaggaggggaguaauaucugucuuacccgcacagaccgagggugguauugcgauaaugcg
ggguccguaagcuucuucccccaagcggagacuugcaagguacagaguaaccgagucuucugcgauacuaugaauaguc
gcacacucccaagcgagguaaauuugugcaacguugauauauuuaaucccaaauacgacuguaagauuaugacuuccaag
acagacgugucuaguucugucauuacuucucucggugcaauuguuuccuguuaugggaagacaaaguguacuggucaa
auaagugucgaggcauaauaaaaacuuucucaaauggaugcgacuacgucagcaauaaagggguugacacgguuucagu
ggguaauacccuuuacuaugucaacaagcaagaaggaaagagcuuguaugucaagggagagccaaucaucaauuucuau
gacccccuuguauuucccucugaugaguucgacgcuuccauaagccaggugaaugaaaaaauuaaucaaagucuggcuu
ucauucggaaaucagacgaacuuuuguccggcggaaguucuggcucuagugggggaagcaugcagauuuaugagggcaa
gcucacagcagaaggcuugagauucggcauuguagccucucgcuucaaccacgcacucguugaccgccuugucgaaggu
gcuauugacuguaucguucggcaugggggucgcgaggaagauauaacucugguuagaguuccugguaguugggagauc
cccguugcggcaggagagcuggcuagaaaagaagacaucgacgcaguaaucgccaucggcguauugauucgcggggcua
ccccgcauuucgauuacauagcgucugaaguaaguaagggacuggcaaaucucucauuggaguugcgaaagccgauuac
uuuuggagucaucaccgccgacacccuggaacaagccauugaacgggggguaccaagcaugguaauaagggcugggaa
gcggcgcucagugccauugaaauggcgaaccuguuuaaaucacugaggugaugauaauaggacuaguggauccaacugg
agacugggugaaagugacuaccagaaagugaggaagccuaaauaaaccuagcguacguaaaaaauggaaagaaccuagcg
uacgaaaaaaaaaaaaaaaaaaaaaaaaaaaaaaaaaaaaaaaaaaaaaaaaaaaaaaaaaaaaaaaaaaaaaaaaaaaaaa
RSV A F Protein (Full-Length)
SEQ ID NO: 19
MELLILKANAITTILTAVTFCFASGQNITEEFYQSTCSAVSKGYLSALRTGWYTSVITI
ELSNIKENKCNGTDAKVKLIKQELDKYKNAVTELQLLMQSTPPTNNRARRELPRFM
NYTLNNAKKTNVTLSKKRKRRFLGFLLGVGSAIASGVAVSKVLHLEGEVNKIKSALL
STNKAVVSLSNGVSVLTSKVLDLKNYIDKQLLPIVNKQSCSISNIETVIEFQQKNNRLL
EITREFSVNAGVTTPVSTYMLTNSELLSLINDMPITNDQKKLMSNNVQIVRQQSYSIM
SIIKEEVLAYVVQLPLYGVIDTPCWKLHTSPLCTTNTKEGSNICLTRTDRGWYCDNA
GSVSFFPQAETCKVQSNRVFCDTMNSLTLPSEINLCNVDIFNPKYDCKIMTSKTDVSS
SVITSLGAIVSCYGKTKCTASNKNRGIIKTFSNGCDYVSNKGMDTVSVGNTLYYVNK
QEGKSLYVKGEPIINFYDPLVFPSDEFDASISQVNEKINQSLAFIRKSDELLHNVNAGK
STTNIMITTIIIVIIVILLSLIAVGLLLYCKARSTPVTLSKDQLSGINNIAFSN
PRKACB 5′ UTR
SEQ ID NO: 20
auucugcuguuugcuccuugccagguucaac
GOT1 5′ UTR
SEQ ID NO: 21
aaaaucucuugauuccuagucucucgau
GUSB 5′ UTR
SEQ ID NO: 22
auccucaaccaagcgccgcagacgguggccgagcgggggaccgggaagc
HSDB 5′ UTR
SEQ ID NO: 23
gucccgcagucggcguccagggcucugcuuguuguguguguguguugcaggccuuauu
CHIT1 3′ UTR
SEQ ID NO: 24
gucgcuaaagccccuccagucccagcuuugaggcugggcccaggaucacucuacagccugccuccuggguuuucccugg
gggccgcaaucuggcuccugcaggccuuucuguggucuuccuuuauccaggcuuucugcucucagccuugccuuccuu
uuuucugcgacuccugggcugccccuuucacuugcaaaauaaa
PKLR 3′ UTR
SEQ ID NO: 25
gacgccccucccuccucuggagucuacguucuccagcccacaccccuccaaagccccaccuuuaaguccucucuucucua
uuccugacccucccuaccugaggccuaucugagacuauaacugucaucuagccccuucgagguugccccuuccccaucuc
cauuucacacagguccugaaagucuguguccaauuaugcacuggccacccaacagcaccaauuguacauucccugcaucc
aaucugcucagcaggcccuaagaugccuugagucuuuaauccca
Albumin 3′ UTR
SEQ ID NO: 26
gcaucacauuuaaaagcaucucagccuaccaugagaauaagagaaagaaaaugaagaucaauagcuuauucaucucuuuu
ucuuuuucguugguguaaagccaacacccugucuaaaaaacauaaauuucuuuaaucauuuugccucuuuucucugugc
uucaauuaauaaaaaauggaaagaaccu
Linker 2
SEQ ID NO: 27
GGGS
Linker 3
SEQ ID NO: 28
GGGGS
Linker 4
SEQ ID NO: 29
GGSG
Linker 5
SEQ ID NO: 30
GSGG
Linker 6
SEQ ID NO: 31
SGGG
Linker 7
SEQ ID NO: 32
SSGG
Linker 8
SEQ ID NO: 33
SSSG
Linker 9
SEQ ID NO: 34
SAGS
Linker 10
SEQ ID NO: 35
GGGSG
Linker 11
SEQ ID NO: 36
TGGGGSGGGGS
Linker 12
SEQ ID NO: 37
GGGGSGGGGS
RSV F Protein F0 Chain
SEQ ID NO: 38
QNITEEFYQSTCSAVSKGYLSALRTGWYTSVITIELSNIKENKCNGTDAKVKLIKQELD
KYKNAVTELQLLMQSTPPTNNRARRELPRFMNYTLNNAKKTNVTLSKKRKRRFLGF
LLGVGSAIASGVAVSKVLHLEGEVNKIKSALLSTNKAVVSLSNGVSVLTSKVLDLKNY
IDKQLLPIVNKQSCSISNIETVIEFQQKNNRLLEITREFSVNAGVTTPVSTYMLINSELL
SLINDMPITNDQKKLMSNNVQIVRQQSYSIMSIIKEEVLAYVVQLPLYGVIDTPCWKL
HTSPLCTTNTKEGSNICLTRTDRGWYCDNAGSVSFFPQAETCKVQSNRVFCDTMNSL
TLPSEINLCNVDIFNPKYDCKIMTSKTDVSSSVITSLGAIVSCYGKTKCTASNKNRGIIK
TFSNGCDYVSNKGMDTVSVGNTLYYVNKQEGKSLYVKGEPIINFYDPLVFPSDEFDA
SISQVNEKINQSLAFIRKSDELLHNVNAGKSTTNIMITTIIIVIIVILLSLIAVGLLLYCKA
RSTPVTLSKDQLSGINNIAFSN
RSV F Protein F1 Chain
SEQ ID NO: 39
FLGFLLGVGSAIASGVAVSKVLHLEGEVNKIKSALLSTNKAVVSLSNGVSVLTSKVLD
LKNYIDKQLLPIVNKQSCSISNIETVIEFQQKNNRLLEITREFSVNAGVTTPVSTYMLT
NSELLSLINDMPITNDQKKLMSNNVQIVRQQSYSIMSIIKEEVLAYVVQLPLYGVIDTP
CWKLHTSPLCTTNTKEGSNICLTRTDRGWYCDNAGSVSFFPQAETCKVQSNRVFCDT
MNSLTLPSEINLCNVDIFNPKYDCKIMTSKTDVSSSVITSLGAIVSCYGKTKCTASNKN
RGIIKTFSNGCDYVSNKGMDTVSVGNTLYYVNKQEGKSLYVKGEPIINFYDPLVFPSD
EFDASISQVNEKINQSLAFIRKSDELLHNVNAGKSTTNIMITTIIIVIIVILLSLIAVGLLLY
CKARSTPVTLSKDQLSGINNIAFSN
RSV F Protein F2 Chain
SEQ ID NO: 40
QNITEEFYQSTCSAVSKGYLSALRTGWYTSVITIELSNIKENKCNGTDAKVKLIKQELD
KYKNAVTELQLLMQSTPPTNNRARR
RSV F Protein Signal Peptide
SEQ ID NO: 41
MELLILKANAITTILTAVTFCFASG
RSV F Protein P27 Fragment
SEQ ID NO: 42
ELPRFMNYTLNNAKKTNVTLSKKRKRR
Extended mCHIT1 5′ UTR
SEQ ID NO: 43
aggauugugcugcaucaagcuugccgccacc
Extended CS 3′ UTR
SEQ ID NO: 44
ugaugauaauaggacuaguggauccaacuggagacugggugaaagugacuaccagaaagugaggaagccuaaauaaacc
uagcguacguaaaaaauggaaagaaccuagcguac
DS2-2(A) F1 Chain
SEQ ID NO: 45
GSAIASGVAVCKVLHLEGEVNKIKSALLSTNKAVVSLSGCGVSVLTFKVLDLKNYIDK
QLLPILNKQSCSISNIETVIEFQQKNNRLLEITREFSVNAGVTTPVSTYMLTNSELLSLIN
DMPITNDQKKLMSNNVQIVRQQSYSIMCIIKEEVLAYVVQLPLYGVIDTPCWKLHTSP
LCTTNTKEGSNICLTRTDRGWYCDNAGSVSFFPQAETCKVQSNRVFCDTMNSRTLPS
EVNLCNVDIFNPKYDCKIMTSKTDVSSSVITSLGAIVSCYGKTKCTASNKCRGIIKTFS
NGCDYVSNKGVDTVSVGNTLYYVNKQEGKSLYVKGEPIINFYDPLVFPSDEFDASISQ
VNEKINQSLAFIRKSDELL
DS2-2 F1 Chain
SEQ ID NO: 46
GSAIASGVAVCKVLHLEGEVNKIKSALLSTNKAVVSLSGCGVSVLTFKVLDLKNYIDK
QLLPILNKQSCSIPNIETVIEFQQKNNRLLEITREFSVNAGVTTPVSTYMLTNSELLSLIN
DMPITNDQKKLMSNNVQIVRQQSYSIMCIIKEEVLAYVVQLPLYGVIDTPCWKLHTSP
LCTTNTKEGSNICLTRTDRGWYCDNAGSVSFFPQAETCKVQSNRVFCDTMNSRTLPS
EVNLCNVDIFNPKYDCKIMTSKTDVSSSVITSLGAIVSCYGKTKCTASNKCRGIIKTFS
NGCDYVSNKGVDTVSVGNTLYYVNKQEGKSLYVKGEPIINFYDPLVFPSDEFDASISQ
VNEKINQSLAFIRKSDELL
DS2-2(A) F2 Chain
SEQ ID NO: 47
QNITEEFYQSTCSAVSKGYLSALRTGWYTSVITIELSNIKENKCNGTDAKVKLIKQELD
KYKNAVTELQLLMQSTPAT
DS2-2 F2 Chain
SEQ ID NO: 48
QNITEEFYQSTCSAVSKGYLGALRTGWYTSVITIELSNIKENKCNGTDAKVKLIKQEL
DKYKNAVTDLQLLMQSTPAT
DS-Cav1
SEQ ID NO: 49
MELLILKANAITTILTAVTFCFASGQNITEEFYQSTCSAVSKGYLSALRTGWYTSVITIE
LSNIKENKCNGTDAKVKLIKQELDKYKNAVTELQLLMQSTPATNNRARRELPRFMN
YTLNNAKKTNVTLSKKRKRRFLGFLLGVGSAIASGVAVCKVLHLEGEVNKIKSALLS
TNKAVVSLSNGVSVLTFKVLDLKNYIDKQLLPILNKQSCSISNIETVIEFQQKNNRLLEI
TREFSVNAGVTTPVSTYMLTNSELLSLINDMPITNDQKKLMSNNVQIVRQQSYSIMCII
KEEVLAYVVQLPLYGVIDTPCWKLHTSPLCTTNTKEGSNICLTRTDRGWYCDNAGSV
SFFPQAETCKVQSNRVFCDTMNSLTLPSEVNLCNVDIFNPKYDCKIMTSKTDVSSSVI
TSLGAIVSCYGKTKCTASNKNRGIIKTFSNGCDYVSNKGVDTVSVGNTLYYVNKQEG
KSLYVKGEPIINFYDPLVFPSDEFDASISQVNEKINQSLAFIRKSDELL
Foldon
SEQ ID NO: 50
GYIPEAPRDGQAYVRKDGEWVLLSTFL
Ferritin
SEQ ID NO: 51
DIEKLLNEQVNKEMQSSNLYMSMSSWCYTHSLDGAGLFLFDHAAEEYEHAKKLIIFL
NENNVPVQLTSISAPEHKFEGLTQIFQKAYEHEQHISESINNIVDHAIKSKDHATFNFLQ
WYVAEQHEEEVLFKDILDKIELIGNENHGLYLADQYVKGIAKSRKS
Ferritin with bullfrog leader
SEQ ID NO:52
ESQVRQQFSKDIEKLLNEQVNKEMQSSNLYMSMSSWSYTHSLDGAGLFLFDHAAEE
YEHAKKLIIFLNENNVPVQLTSISAPEHCFEGLTQIFQKAYEHEQHISESINNIVDHAIKS
KDHATFNFLQWYVAEQHEEEVLFKDILDKIELIGNENHGLYLADQYVKGIAKSRKS
Beta-annulus
SEQ ID NO: 53
INHVGGTGGAIMAPVAVTRQLVGS
DS-Cav1 with transmembrane domain
SEQ ID NO: 54
MELLILKANAITTILTAVTFCFASGQNITEEFYQSTCSAVSKGYLSALRTGWYTSVITIE
LSNIKENKCNGTDAKVKLIKQELDKYKNAVTELQLLMQSTPATNNRARRELPRFMN
YTLNNAKKTNVTLSKKRKRRFLGFLLGVGSAIASGVAVCKVLHLEGEVNKIKSALLS
TNKAVVSLSNGVSVLTFKVLDLKNYIDKQLLPILNKQSCSISNIETVIEFQQKNNRLLEI
TREFSVNAGVTTPVSTYMLTNSELLSLINDMPITNDQKKLMSNNVQIVRQQSYSIMCII
KEEVLAYVVQLPLYGVIDTPCWKLHTSPLCTTNTKEGSNICLTRTDRGWYCDNAGSV
SFFPQAETCKVQSNRVFCDTMNSLTLPSEVNLCNVDIFNPKYDCKIMTSKTDVSSSVI
TSLGAIVSCYGKTKCTASNKNRGIIKTFSNGCDYVSNKGVDTVSVGNTLYYVNKQEG
KSLYVKGEPIINFYDPLVFPSDEFDASISQVNEKINQSLAFIRKSDELLHNVNAVKSTTN
IMITTIIIVIIVILLSLIAVGLLLYCKARSTPVTLSKDQLSGINNIAFSN

1. A respiratory syncytial virus (RSV) messenger RNA (mRNA) vaccine comprising an mRNA molecule encoding a nanoparticle subunit comprising an immunogen and a scaffold joined by a linker, wherein the linker is 9 to 13 amino acids in length, the scaffold comprises a lumazine synthase, and the immunogen comprises a modified RSV F protein comprising a deletion of RSV F protein positions 104-144, a GS peptide linker between RSV F protein positions 103 and 145, and the following mutations: S155C, N183GC, S190F, V207L, S290C, L373R and N428C.

2. The RSV mRNA vaccine of claim 1, wherein, when assembled, the nanoparticle comprises about 60 nanoparticle subunits.

3. The RSV mRNA vaccine of claim 1, wherein the lumazine synthase comprises the amino acid sequence set forth in SEQ ID NO: 3, or a variant thereof having at least 90% identity to SEQ ID NO: 3.

4. The RSV mRNA vaccine of claim 1, wherein the linker is 11 amino acids in length.

5. (canceled)

6. The RSV mRNA vaccine of claim 1, wherein the nanoparticle subunit comprises, from N-terminus to C-terminus, the immunogen, the linker and the scaffold.

7. The RSV mRNA vaccine of claim 6, wherein the linker and scaffold together (linker-scaffold) comprise the amino acid sequence set forth in SEQ ID NO: 5.

8. The RSV mRNA vaccine of claim 1, wherein the immunogen is a modified RSV F protein comprising an F1 polypeptide and an F2 polypeptide.

9. The RSV mRNA vaccine of claim 1, wherein the modified RSV F protein further comprises one or more of the following substitutions: S46G, E92D and S215P.

10. (canceled)

11. The RSV mRNA vaccine of claim 1, wherein the immunogen comprises the amino acid sequence set forth in SEQ ID NO: 1.

12. The RSV mRNA vaccine of claim 1, wherein the nanoparticle subunit comprises the amino acid sequence set forth in SEQ ID NO: 6, or a variant thereof having at least 90% identity to SEQ ID NO: 6.

13. (canceled)

14. The RSV mRNA vaccine of claim 1, wherein the immunogen comprises the amino acid sequence set forth in SEQ ID NO: 2.

15. The RSV mRNA vaccine of claim 14, wherein the nanoparticle subunit comprises the amino acid sequence set forth in SEQ ID NO: 7, or a variant thereof having at least 90% identity to SEQ ID NO: 7.

16. (canceled)

17. The RSV mRNA vaccine of claim 1, wherein the RSV F protein amino acid numbering is according to the reference sequence set out in SEQ ID NO: 19.

18. The RSV mRNA vaccine of claim 1, wherein the mRNA molecule comprises:
(i) an immunogen coding sequence comprising the nucleotide sequence set forth in SEQ ID NO: 8 or SEQ ID NO: 9, or a sequence having at least 90% identity thereto;
(ii) a scaffold coding sequence comprising the nucleotide sequence set forth in SEQ ID NO: 10, or a sequence having at least 90% identity thereto; and
(iii) a linker coding sequence comprising the nucleotide sequence set forth in SEQ ID NO: 11, or a sequence having at least 90% identity thereto.

19. The RSV mRNA vaccine of claim 18, wherein the mRNA molecule comprises nanoparticle subunit coding sequence comprising the nucleotide sequence set forth in SEQ ID NO: 13 or SEQ ID NO: 14.

20. The RSV mRNA vaccine of claim 1, wherein the mRNA molecule comprises a 5′ UTR comprising the nucleotide sequence set forth in SEQ ID NO: 15, or a sequence having at least 90% identity thereto.

21. The RSV mRNA vaccine of claim 1, wherein the mRNA molecule comprises a 3′ UTR comprising the nucleotide sequence set forth in SEQ ID NO: 16, or a sequence having at least 90% identity thereto.

22. The RSV mRNA vaccine of claim 1, wherein the mRNA molecule comprises the nucleotide sequence set forth in SEQ ID NO: 17 or SEQ ID NO: 18, or a sequence having at least 90% identity thereto.

23-24. (canceled)

25. The RSV mRNA vaccine of claim 1, wherein the mRNA molecule comprises pseudouridine or modified pseudouridine nucleotides.

26. (canceled)

27. The RSV mRNA vaccine of claim 1, wherein the mRNA comprises a 5′ cap having a Cap1 or modified Cap1 structure.

28. The RSV mRNA vaccine of claim 1, wherein the mRNA molecule is formulated in a lipid nanoparticle.

29-33. (canceled)

34. A method of inducing an immune response in a subject, comprising administering to the subject the RSV mRNA vaccine of claim 1.

35. A method of preventing a lower respiratory tract disease (LRTD) caused by respiratory syncytial virus (RSV) in a subject, comprising administering to the subject the RSV mRNA vaccine of claim 1.

36-37. (canceled)

38. An expression vector comprising an expression cassette encoding an mRNA molecule as defined in claim 1.

39. A cell comprising the expression vector of claim 38.

40. A method of manufacturing an mRNA molecule as defined in claim 1, comprising expressing the mRNA from an expression vector comprising an expression cassette encoding the mRNA molecule, optionally wherein the mRNA is expressed by in vitro transcription.

41. A protein nanoparticle comprising a multimer of a nanoparticle subunit as defined in claim 1, preferably a 60-mer of the nanoparticle subunit.
